(19) United States
(12) Patent Application Publication
(10) Pub. No.: US 2025/0281613 A1
(43) Pub. Date: Sep. 11, 2025

(54) ENHANCING SAFETY OF T-CELL-MEDIATED IMMUNOTHERAPY

(71) Applicant: CELLECTIS S.A., PARIS (FR)

(72) Inventors: Shipra DAS, New York, NY (US); Julien VALTON, Paris (FR); Laurent POIROT, Paris (FR); Philippe DUCHATEAU, Paris (FR)

(21) Appl. No.: 18/861,400

(22) PCT Filed: Jun. 30, 2023
(86) PCT No.: PCT/EP2023/067961

§ 371 Date: Oct. 29, 2024

Related U.S. Application Data
(60) Provisional application No. 63/367,363, filed on Jun. 30, 2022.

(30) Foreign Application Priority Data
Jul. 18, 2022 (DK) PA202270384

(51) Int. Cl. A61K 40/42; A61K 40/11; A61K 40/31; A61P 35/00; C07K 14/725; C07K 16/30; C07K 16/40

(52) U.S. Cl. CPC A61K 40/4249; A61K 40/11; A61K 40/31; A61K 40/4255; A61P 35/00; C07K 14/7051; C07K 16/30; C07K 16/40; A61K 2239/23

(57) ABSTRACT

This document relates to engineered immune cells comprising a FAP-CAR and a tumor-CAR with differential expressions, their use in the treatment of tumors expressing FAP, as well as methods and materials for the preparation thereof.

### FIELD OF THE INVENTION

The present document generally relates to the field of cancer, in particular, cell therapies and immunotherapies for the treatment of solid tumors or haematological cancers characterized by the presence of FAP in the tumor microenvironment, in patients.

### BACKGROUND

Adoptive cell therapy, also known as cellular immunotherapy, is a form of treatment that uses the cells of the immune system to eliminate pathological cells, such as infected or malignant cells. Some of these approaches involve directly isolating a person's own immune cells and simply expanding their numbers, whereas others involve genetically engineering immune cells from patients (autologous approach) or donors (allogeneic approach) to boost and/or redirect them towards specific target tissues. In the case of cancer, immune cells, especially immune cytolytic and helper T-lymphocytes, Natural Killers and Macrophages, are particularly powerful against cancer, due to their ability to bind to markers known as antigens on the surface of cancer cells. Cellular immunotherapies take advantage of this natural ability and can be deployed in different ways: Tumor-Infiltrating Lymphocyte (TIL) therapy, Engineered T Cell Receptor (TCR) cell therapy, Chimeric Antigen Receptor (“CAR”) immune cell therapy, and Natural Killer (NK) cell therapy.

Chimeric antigen receptors-expressing immune cells are cells that have been genetically engineered to express chimeric antigen receptors (CARs) usually designed to recognize specific tumor antigens and kill cancer cells that express said tumor antigen(s). These are generally T-cells expressing CARs (“CAR-T cells”), Natural Killer cells expressing CARs (“CAR-NK cells”), or macrophages expressing CARs.

CARs are synthetic receptors consisting of a targeting moiety that is associated with one or more signalling domains in a single or multiple fusion molecule(s). In general, the binding moiety of a CAR can include of an antigen-binding domain of a single-chain antibody (scFv), comprising the light and heavy variable fragments of a monoclonal antibody joined by a flexible linker. Binding moieties based on receptor or ligand domains have also been used successfully. The signalling domains for first generation CARs are derived from the cytoplasmic region of the CD3zeta (or CD3ζ) or the Fc receptor gamma chains. First generation CARs have been shown to successfully redirect T-cell cytotoxicity, however, they failed to provide prolonged expansion and anti-tumor activity in vivo. Signalling domains from co-stimulatory molecules including CD28, OX-40 (CD134), ICOS, and 4-1BB (CD137) have been added alone (second generation) or in combination (third generation) to enhance survival and increase proliferation of CAR modified T-cells. CARs have successfully allowed T-cells to be redirected against antigens expressed at the surface of tumor cells from various malignancies including lymphomas and solid tumors (Jena, Dotti et al. Blood (2010) 116 (7): 1035-44).

Adoptive immunotherapy, which involves the transfer of autologous or allogeneic antigen-specific T-cells generated ex vivo, is a promising strategy to treat viral infections and cancer as confirmed by the increase in the number of clinical trials involving CAR-T cells.

So far, only autologous CAR T-cells have been approved by the US Food and Drug Administration (FDA) (e.g. Novartis' anti-CD19 CAR-T tisagenlecleucel (Kymriah™) for the treatment of precursor B-cell acute lymphoblastic leukemia; Kite Pharma's anti-CD19 CAR-T axicabtagene ciloleucel (Yescarta™) for certain types of large B-cell lymphoma in adult patients expressing CD19 as a marker, anti-BCMA CAR-T idecabtagene vicleucel (Abecma™) for treatment of adult patients with relapsed or refractory multiple myeloma, and anti-CD19 CAR-T lisocabtagene maraleucel (Breyanzi™) for adult patients with relapsed or refractory large B-cell lymphoma, anti-CD19 CAR-T brexucabtagene autoleucel (Tecartus™) for patients with relapsed or refractory mantle cell lymphoma). Allogeneic approaches are more challenging due to the alloreactivity of the cells with respect to the patient's own immune cells. The most advanced programs involve inactivating endogenous T-cell receptor genes by using specific rare-cutting endonucleases, such as TALE-nucleases, to reduce the alloreactivity of the cells prior to administering them to patients as reported by Poirot et al. (Cancer. Res. (2015) 75 (18): 3853-3864) and Qasim et al. (Science Translational (2017) 9 (374)).

Meanwhile, inactivation of TCR (e.g. TRAC and/or TRBC) in primary T-cells can be combined with the inactivation of MHC components such as beta-2-microglobulin (B2M) and/or with inactivation of genes encoding checkpoint proteins, such as described for instance in WO 2014/184744.

T-cell mediated anti-tumor cytotoxicity is a promising immunotherapeutic strategy for both leukemia and solid tumors. However, several factors limit the efficacy of tumor-antigen targeted CAR-T therapy against solid tumors, including lack of tumor-infiltrating lymphocytes (TIL) and an immune suppressive tumor microenvironment (TME) (Stern et al. (2020) Cancer Treat Res. 180:297-326). Most solid tumor microenvironments are characterized by the presence of activated fibroblasts called cancer-associated fibroblasts (CAFs) that express unique surface proteins such as FAP (Kalluri R. Nat Rev Cancer (2016) 16:582-98). CAFs can inhibit TILs and promote immune suppression (Wang et al. (2014) Cancer Immunol Res. 2:154-66).

More recently, Sakemura et al. (Blood (2019) 134 (suppl.1) 135; Biol. Blood Marrow Transplant (2019) 26 (S224)) have shown that dual targeting of both malignant plasma cells and the CAFs within the tumor microenvironment overcomes resistance to CAR-T cell therapy in multiple myeloma.

The treatment of cancer, particularly treatment of cancers characterized by solid tumors remains a great challenge in healthcare. What is needed are new compositions and treatments that are both effective against solid tumors and safe in patients. More particularly needed are new “universal” compositions and treatments that are useful in treating solid tumors in all patients without any allogeneic limitations as, generally, the patients are not the donors of the cells from which said compositions have been prepared. Also particularly needed are new compositions and treatments that target the tumor tissues but have a limited effect on healthy tissues that may also express the target antigen.

This background information is provided for informational purposes only. No admission is necessarily intended, nor should it be construed, that any of the preceding information constitutes prior art against the present invention.

### SUMMARY

This document provides methods and materials for treating cancer. For example, this document provides cells (e.g., immune cells such as T cells or NK cells engineered to express CARs having the ability to bind to a FAP polypeptide expressed by a cancer cell (e.g., a cancer cell of a solid tumor or a cancer cell of a haematological cancer) and/or expressed by a cell present within a tumor microenvironment.

It is to be understood that both the foregoing general description of the embodiments and the following detailed description are exemplary, and thus do not restrict the scope of the embodiments.

The methods and materials provided herein are particularly suited for treatment of cancers characterized by the presence of FAP in their tumor microenvironment. The methods and materials provided herein also are particularly suited to achieve a “universal” treatment, where the components of the treatment can be used in many unrelated patients.

In general, one aspect of this document features an engineered cell comprising:
a) an exogenous nucleic acid sequence encoding a chimeric antigen receptor (CAR) targeting the Fibroblast activation Protein (FAP) (“FAP-CAR”) placed under the transcriptional control of an exogenous or endogenous constitutive promoter; and
b) an exogenous nucleic acid sequence encoding a chimeric antigen receptor (CAR) targeting a tumor antigen (“tumor-CAR”) placed under the transcriptional control of an exogenous or endogenous inducible promoter;
wherein said exogenous nucleic acid sequences a) and b) are integrated in the cell's genome, and
wherein the expression of the tumor-CAR is inducible upon activation of the cell.

Said engineered cell can be an immune cell such as a T-cell, a NK-cell, or a macrophage.

In some cases, said engineered cell can be an engineered T-cell or engineered NK-cell.

In some cases, said engineered cell can be an iPSC that may be an intermediate in the production of an engineered immune cell, such as a T-cell, a NK-cell, or a macrophage, as described herein. In some cases, the engineered cell can be an engineered immune cell that derives from the engineered iPSC after that said iPSC has been submitted to one or more differentiation steps.

Although the various aspects described herein can apply to a situation where the cells are immune cells such as T-cells, these various aspects similarly apply to NK-cells and macrophages and are, thus, included herein.

In some cases, an engineered cell described herein can be a T cell that was genetically modified to suppress or repress expression of a T-cell receptor (TCR) (e.g., an endogenous TCR) by inactivation of a gene encoding a TCR component (e.g., a TRAC gene and/or TRBC gene) and, optionally, to suppress or repress expression of at least one gene controlling MHC complex surface presentation, such as B2M and class II major histocompatibility complex transactivator (CIITA), and, optionally, to suppress or repress expression of CD52, and, optionally, to suppress or repress expression of at least one immune checkpoint or receptor thereof, in the T-cell.

In another aspect, this document features an engineered T-cell comprising:
a) an exogenous nucleic acid sequence encoding a chimeric antigen receptor (CAR) targeting the Fibroblast activation Protein (FAP) (“FAP-CAR”) placed under the transcriptional control of an exogenous or endogenous constitutive promoter; and
b) an exogenous nucleic acid sequence encoding a chimeric antigen receptor (CAR) targeting a tumor antigen (“tumor-CAR”) placed under the transcriptional control of an exogenous or endogenous inducible promoter;
wherein said exogenous nucleic acid sequences of a) and b) are integrated in the cell's genome,
wherein the expression of the tumor-CAR is inducible upon activation of said T-cell.

In some cases, the FAP-CAR can comprise:
(a1) an extracellular FAP-binding-domain comprising VH and VL amino acid sequences from a monoclonal anti-FAP antibody,
(a2) a hinge selected from a FcγRIII hinge, a CD8a hinge, and an IgG1 hinge,
(a3) a transmembrane domain comprising a CD8a transmembrane domain or a CD28 transmembrane domain, and
(a4) a cytoplasmic domain comprising a CD3 zeta signaling domain, and optionally a co-stimulatory domain from 4-1BB or from CD28.

In some cases, the tumor-CAR can comprise:
(b1) an extracellular tumor antigen-binding-domain comprising VH and VL amino acid sequences from a monoclonal anti-tumor antigen antibody,
(b2) a hinge selected from a FcγRIII hinge, a CD8α hinge, and an IgG1 hinge,
(b3) a transmembrane domain comprising a CD8α transmembrane domain or a CD28 transmembrane domain, and
(b4) a cytoplasmic domain comprising a CD3 zeta signaling domain and a co-stimulatory domain from 4-1BB or from CD28.

In some cases, the constitutive promoter can be selected from the group consisting of the promoters of EF1A, CD52, GAPDH, CMV, hPGK, UBC, SV40, PGK, CAGG, TRAC, TRBC, TRGC, TRDC, B2M, CD5, CS1, CD45, RPBSA, CD4, and CD8; and/or the inducible promoter can be selected from the group consisting of NFAT responsive elements, and the promoters of PDCD1, CD25, TIM3, TIGIT, CCL1, NR4A3, EGR3, GOS2, IL22, RGS16, FASLG, RDH10, CSF1, GM-CSF, LAG3, CTLA-4, IL10, NUR77, and FOXP3.

In some cases, the constitutive promoter can be selected from the group consisting of the promoters of EF1A, TRAC, B2M, CD52, CS1, CD45, CD5, and GAPDH. For example, the constitutive promoter can be a EF1A, TRAC, CD52, or B2M promoter. For example, the constitutive promoter can be an EF1A promoter.

In some cases, the inducible promoter can be selected from the group consisting of NFAT responsive elements, and the promoters of PDCD1, CD25, GM-CSF, TIM3, and TIGIT. For example, the inducible promoter can be a PDCD1 promoter.

In some cases, the constitutive promoter can be an endogenous TRAC promoter or an exogenous EF1A promoter, and the inducible promoter can be an endogenous PDCD1 promoter.

In some cases, the constitutive promoter can be an exogenous EF1A promoter, and the inducible promoter can be an endogenous PDCD1 promoter.

In some cases, the FAP-CAR can comprise an extracellular FAP-binding-domain comprising the amino acid sequence of SEQ ID NO: 9, SEQ ID NO: 20, SEQ ID NO: 31, or SEQ ID NO: 42, and/or the tumor-CAR can comprise an extracellular tumor antigen-binding-domain comprising the amino acid sequence of SEQ ID NO: 53, SEQ ID NO: 61, or SEQ ID NO: 69.

In some cases, the FAP-CAR can comprise an extracellular FAP-binding-domain comprising the amino acid sequence of SEQ ID NO: 9 and the tumor-CAR can comprise an extracellular tumor antigen-binding-domain comprising the amino acid sequence of SEQ ID NO: 61 or SEQ ID NO: 69.

In some cases, an engineered T-cell described herein can be a T-cell that was genetically modified to suppress or repress expression of a T-cell receptor (TCR) (e.g., an endogenous TCR) by inactivation of a gene encoding a TCR component (e.g., a TRAC and/or TRBC) and, optionally, to suppress or repress expression of at least one gene controlling MHC complex surface presentation, such as B2M and CIITA, and, optionally, to suppress or repress expression of CD52, and, optionally, to suppress or repress expression of at least one immune checkpoint or receptor thereof, in the T-cell.

In some cases, the engineered T-cell described herein can be a T-cell that was genetically modified (i) to suppress or repress expression of a T-cell receptor (TCR) by inactivation of a gene encoding a TCR component (e.g., a TRAC and/or TRBC), (ii) to suppress or repress expression of PDCD1 or the gene encoding the receptor of PD1, and optionally (iii) to suppress or repress expression of B2M, and optionally (iv) to suppress or repress expression of CD52, in the T-cell.

In another aspect, this document features a method of treatment of a cancer characterized by the presence of FAP in the tumor microenvironment comprising administering a therapeutically effective amount of engineered immune cells (e.g., engineered T-cells) comprising (a) an exogenous nucleic acid sequence encoding a FAP-CAR placed under the transcriptional control of an exogenous or endogenous constitutive promoter; and (b) an exogenous nucleic acid sequence encoding a tumor-CAR placed under the transcriptional control of an exogenous or endogenous inducible promoter as described herein.

In another aspect, this document features a pharmaceutical composition comprising a therapeutically effective amount of engineered immune cells (e.g., engineered T-cells) as described herein.

In another aspect, this document features a composition comprising a therapeutically effective amount of engineered immune cells (e.g., engineered T-cells) as described herein, for use in the treatment of a cancer characterized by the presence of FAP in the tumor microenvironment.

In some cases, the FAP-CAR can be constitutively expressed in the engineered immune cells (e.g., engineered T-cells) either through lentiviral integration or through nuclease-mediated cDNA insertion at one or more constitutively expressed gene loci such as one or more of the TRAC, B2M, or CD52 gene loci.

In some cases, the TRAC and/or B2M gene loci can be disrupted, for instance by TALE-Nuclease, to inhibit graft versus host disease (GvHD) and increase the engineered immune cells' persistence (e.g., an engineered T cells' persistence) in an allogeneic setting.

In some cases, the tumor-CAR can be inducible upon activation of the engineered immune cell (e.g., engineered T-cell) and can be encoded by an exogenous nucleic acid sequence that is incorporated in the cell's genome either through lentiviral integration or through nuclease-mediated cDNA insertion at one or more inducible gene loci such as one or more of the PDCD1, CD25, GM-CSF, TIM3, and TIGIT gene loci; for instance, at the PDCD1 inducible locus.

In some cases, the tumor-antigen targeted by said tumor-CAR can be selected from the group consisting of MUC1 (e.g. human MUC1), Mesothelin (e.g. human Mesothelin), EGFR (e.g. human EGFR), VEGF (e.g. human VEGF), and Trop2 (e.g. human Trop2).

In some cases, the tumor-antigen targeted by said tumor-CAR can be MUC1 (e.g. human MUC1) or Mesothelin (e.g. human Mesothelin).

Other embodiments relate to the situation where the engineered immune cell(s) are NK-cells, thus, relate, for instance, to an engineered NK-cell comprising:
a) an exogenous nucleic acid sequence encoding a FAP-CAR placed under the transcriptional control of an exogenous or endogenous constitutive promoter; and
b) an exogenous nucleic acid sequence encoding a tumor-CAR placed under the transcriptional control of an exogenous or endogenous inducible promoter;
wherein said exogenous nucleic acid sequences a) and b) are integrated in the cell's genome;
wherein the expression of the tumor-CAR is inducible upon activation of said NK-cell.

In some cases, the FAP-CAR can comprise:
(a1) an extracellular FAP-binding-domain comprising VH and VL amino acid sequences from a monoclonal anti-FAP antibody,
(a2) a hinge selected from a FcγRIII hinge, a CD8 hinge, and an IgG1 hinge,
(a3) a transmembrane domain comprising a CD8α transmembrane domain or a CD28 transmembrane domain, and
(a4) a cytoplasmic domain comprising a CD3 zeta signaling domain, and optionally a co-stimulatory domain from 4-1BB or from CD28.

In some cases, the tumor-CAR can comprise:
(b1) an extracellular tumor antigen-binding-domain comprising VH and VL amino acid sequences from a monoclonal anti-tumor antigen antibody,
(b2) a hinge selected from a FcγRIII hinge, a CD8α hinge, and an IgG1 hinge,
(b3) a transmembrane domain comprising a CD8α transmembrane domain or a CD28 transmembrane domain, and
(b4) a cytoplasmic domain comprising a CD3 zeta signaling domain and a co-stimulatory domain from 4-1BB or from CD28.

In another aspect, this document features a method of producing a population of cells comprising engineered immune cells (e.g., engineered T-cells) described herein, comprising:
(i) providing immune cells from a donor;
(ii) inactivating the expression of a T-Cell Receptor (TCR) (e.g., an endogenous TCR) in the cells or its presentation at the cell surface in the case of said cells being T-cells;
(iii) integrating in the cells' genome an exogenous nucleic acid sequence encoding a FAP-CAR placed under the transcriptional control of an exogenous or endogenous constitutive promoter; and
(iv) integrating in the cells' genome an exogenous nucleic acid sequence encoding a tumor-CAR placed under the transcriptional control of an exogenous or endogenous inducible promoter;
(v) optionally, isolating the engineered cells that do not express a TCR (e.g., an endogenous TCR) at their cell surface;
wherein the expression of the tumor-CAR is inducible upon activation of the immune cells (e.g., the T-cells).

In another aspect, this document features a method of producing a population of cells comprising engineered NK-cells described herein, comprising:
(i) providing immune cells from a donor;
(ii) integrating in the cells' genome an exogenous nucleic acid sequence encoding a FAP-CAR placed under the transcriptional control of an exogenous or endogenous constitutive promoter; and
(iii) integrating in the cells' genome an exogenous nucleic acid sequence encoding a tumor-CAR placed under the transcriptional control of an exogenous or endogenous inducible promoter;
wherein the expression of the tumor-CAR is inducible upon activation of the NK-cells.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE FIGURES

The skilled artisan will understand that the drawings, described below, are for illustration purposes only. The drawings are not intended to limit the scope of the present teachings in any way.

FIG. 1: A) Strategy for improving safety and efficacy of CAR-T based treatments using a FAP-CAR expressed constitutively and a tumor-CAR expressed under transcriptional control of an inducible promoter. B) Different approaches to deliver FAP-CAR in the cell's genome allowing its expression under control of a constitutive promoter C) Different approaches to deliver tumor-CAR in the cell's genome allowing its expression under control of an inducible promoter.

FIG. 2: A) Strategy for improving safety and efficacy of CAR-T based treatments illustrated in the Examples section. B) FAP-CAR construct is either integrated in the TRAC locus in the cell's genome (TRAC targeted integration) allowing its expression under the control of the endogenous TRAC promoter or is integrated randomly in the cell's genome using a rLV and its expression is under the control of an exogenous EF1alpha promoter. C) Meso-CAR construct is integrated at the PDCD1 locus in the cell's genome (PDCD1 targeted integration). This construct also contains a LNGFR reporter gene for targeted integration enrichment.

FIG. 3: A) Results of flow cytometry showing the percentage of LNGFR positive cells obtained when Meso-CAR is targeted to the PDCD1 locus in cells that are either TRAC knock-out (TRAC_{KO}PDCD1_{MesoCAR}) or that further have a FAP-CAR targeted at the TRAC locus (TRAC_{FAPCAR}PDCD1_{MesoCAR}), before and after LNGFR positive enrichment. B) Percentage of targeted integration of the Meso-CAR measured by ddPCR.

FIG. 4: A) Results of flow cytometry showing TCRalpha/beta (Y-axis) and FAP-CAR staining (X-axis) of mock electroporated (Mock), TRAC knock-out plus PDCD1 knock-out (TRAC_{KO}PDCD1_{KO}), FAP-CAR targeted integration at the TRAC locus plus PDCD1 knock-out (TRAC_{FAPCAR}PDCD1_{KO}), TRAC knock-out plus Meso-CAR targeted integration at PDCD1 locus (TRAC_{KO}PDCD1_{MesoCAR}), or FAP-CAR targeted integration at the TRAC locus plus Meso-CAR targeted integration at PDCD1 locus (TRAC_{FAPCAR}PDCD1_{MesoCAR}) T cells. B) Results of flow cytometry post activation showing PD-1 (Y-axis) and Meso-CAR staining (X-axis) after PMA and Ionomycin induced activation of mock electroporated (Mock), TRAC knock-out plus PDCD1 knock-out (TRAC_{KO}PDCD1_{KO}), FAP-CAR targeted integration at the TRAC locus plus PDCD1 knock-out (TRAC_{FAPCAR}PDCD1_{KO}), TRAC knock-out plus Meso-CAR targeted integration at PDCD1 locus (TRAC_{KO}PDCD1_{MesoCAR}), or FAP-CAR targeted integration at the TRAC locus plus Meso-CAR targeted integration at PDCD1 locus (TRAC_{FAPCAR}PDCD1_{MesoCAR}) T cells.

FIG. 5: Percentage of targeted integration of the Meso-CAR measured by ddPCR in mock electroporated (Mock), TRAC knock-out plus targeted integration of Meso-CAR at PDCD1 locus (TRAC_{KO}PDCD1_{MesoCAR}), or TRAC knock-out plus FAP-CAR random integration plus targeted integration of Meso-CAR at PDCD1 locus (TRAC_{KO}FAPCARr_{LV}PDCD1_{MesoCAR}) T cells, post DLNGFR enrichment.

FIG. 6: Results of flow cytometry showing FAP-CAR (Y-axis) and Meso-CAR (X-axis) staining after recombinant FAP protein induced activation of mock electroporated (Mock), TRAC knock-out plus PDCD1 knock-out (TRAC_{KO}PDCD1_{KO}), FAP-CAR random integration plus TRAC and PDCD1 double knock-out (FAPCARr_{LV}TRAC_{KO}PDCD1_{KO}), TRAC knock-out plus Meso-CAR targeted integration at PDCD1 locus (TRAC_{KO}PDCD1_{MesoCAR}), or FAP-CAR random integration plus TRAC knock-out plus Meso-CAR targeted integration at PDCD1 locus (TRAC_{KO}FAPCARr_{LV}PDCD1_{MesoCAR}) T cells.

FIG. 7: A) Images of HCC70-NL-GFP with CAF cell spheroids treated with mock or the indicated CAR-T cells. Bright cells are the HCC70-NL-GFP tumor cells. B) Images of HCC70-NL-GFP without CAF cells treated with mock or the indicated CAR-T cells. Bright cells are the HCC70-NL-GFP tumor cells.

FIG. 8: RNAseq data for CS1-CAR-T cells at t=0 hours (not activated) or 24 hours post activation using recombinant CS1 protein (open symbols). Closed symbols represent genes selected based on expression level (lower than 100 TPM at 0 hours, greater than 50 TPM at 24 hours, and fold change at 24 hours greater than 5).

FIG. 9: (A) Flow cytometry analysis of human Mesothelin protein expression on Y-axis and human FAP protein expression on X-axis in NCI-H226 and NCI-H226-FAP_{50%} mesothelioma tumor cells. (B) Schematic of in vivo study to assess anti-tumor cytotoxicity and bystander activity of FAP-CAR; TRAC_{KO}PDCD1_{Meso-CAR} T-cells using a bilateral sub-cutaneous mouse model derived from NCI-H226 and NCI-H226-FAP_{50%} tumor cells. (C) Graphical representation of number of CD45⁺ cells per gram tumor in mice treated as indicated, assessed using flow cytometry. (D) Graphical representation of percentage of Meso-CAR-positive cells among CD45⁺ cells in tumors of mice treated as indicated, assessed using flow cytometry. (E) Graph representing growth kinetics of sub-cutaneous NCI-H226-FAP_{50%} tumors in mice treated as indicated over time. P-values determined by Student t test (two-tailed, unpaired), n=5 mice per cohort. (F) Graph representing growth kinetics of sub-cutaneous NCI-H226 tumors in mice treated as indicated over time. P-values determined by Student t test (two-tailed, unpaired), n=5 mice per cohort.

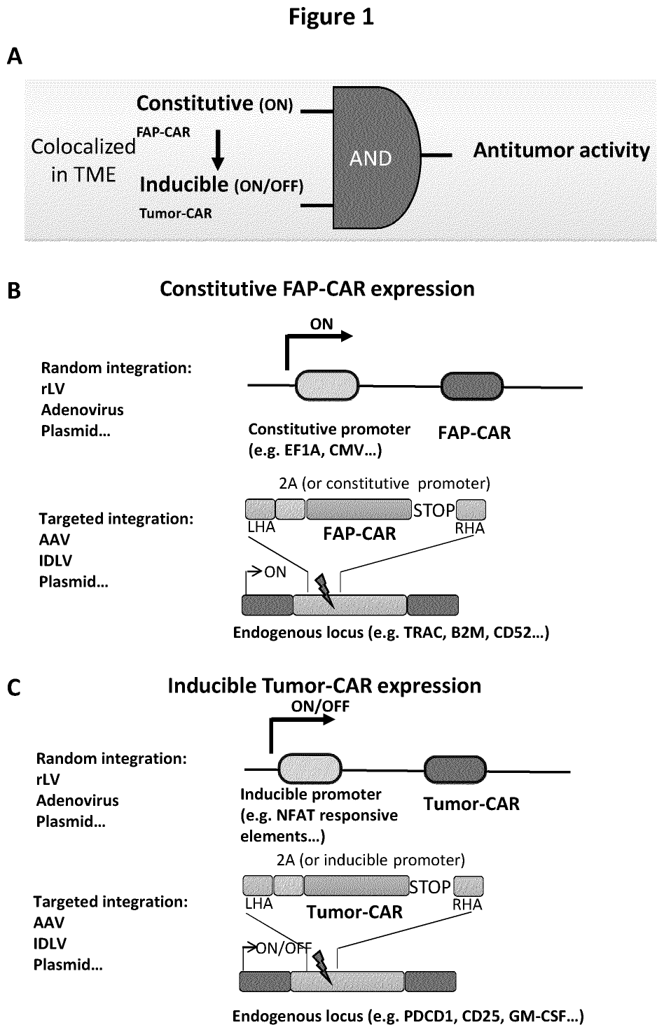

### DETAILED DESCRIPTION

This document provides methods and materials that can be used to harness spatial characteristics of a tumor microenvironment (“TME”) to amplify anti-tumor activity of immunotherapies locally. For example, in some cases, this document provides Fibroblast Activation Protein (“FAP”)-targeting CARs that can be used as a trigger for targeted cell immunotherapy, thereby allowing enhancement of CAR-T tumoricide activity when and where it is needed, and thereby enhancing both the therapeutic effect and the safety of targeted cell immunotherapy in the patients.

For the purpose of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used). The use of “or” means “and/or” unless stated otherwise. As used in the specification and claims, the singular form “a,” “an” and “the” include plural references unless the context clearly dictates otherwise. For example, the term “a cell” includes a plurality of cells, including mixtures thereof. The use of “comprise,” “comprises,” “comprising,” “include,” “includes,” and “including” are interchangeable and not intended to be limiting. Furthermore, where the description of one or more embodiments uses the term “comprising,” those skilled in the art would understand that, in some specific instances, the embodiment or embodiments can be alternatively described using the language “consisting essentially of” and/or “consisting of”.

As used herein, the term “about” means plus or minus 10% of the numerical value of the number with which it is being used.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will prevail. Further, the materials, methods, and examples are illustrative only and are not intended to be limiting, unless otherwise specified.

The practice of the present invention will employ, unless otherwise indicated, techniques of cell biology, cell culture, molecular biology, transgenic biology, microbiology, recombinant DNA, gene editing, and immunology, which belong to the knowledge of the skilled in the art. Such techniques are explained fully in the literature. See, for example, Current Protocols in Molecular Biology (Frederick M. AUSUBEL, 2000, Wiley and son Inc, Library of Congress, USA); Molecular Cloning: A Laboratory Manual, Third Edition, (Sambrook et al, 2001, Cold Spring Harbor, New York: Cold Spring Harbor Laboratory Press); Oligonucleotide Synthesis (M. J. Gait ed., 1984); Mullis et al. U.S. Pat. No. 4,683,195; Nucleic Acid Hybridization (B. D. Harries & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the series, Methods In ENZYMOLOGY (J. Abelson and M. Simon, eds.-in-chief, Academic Press, Inc., New York), specifically, Vols. 154 and 155 (Wu et al. eds.) and Vol. 185, “Gene Expression Technology” (D. Goeddel, ed.); Gene Transfer Vectors For Mammalian Cells (J. H. Miller and M. P. Calos eds., 1987, Cold Spring Harbor Laboratory); Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); and Manipulating the Mouse Embryo, (Cold Spring Harbor Laboratory Press, Cold Spring Harbor, N.Y., 1986).

Unless specifically defined herein, all technical and scientific terms used have the same meaning as commonly understood by a skilled artisan in the fields of gene therapy, biochemistry, genetics, immunology, cancer, molecular biology, and gene editing. Definitions of common terms in molecular biology may be found, for example, in Benjamin Lewin, Genes VII, published by Oxford University Press, 2000 (ISBN 019879276X); Kendrew et al. (eds.); The Encyclopedia of Molecular Biology, published by Blackwell Publishers, 1994 (ISBN 0632021829); and Robert A. Meyers (ed.), Molecular Biology and Biotechnology: a Comprehensive Desk Reference, published by Wiley, John & Sons, Inc., 1995 (ISBN 0471186341).

As used herein, a “recipient” is a patient that receives a transplant, such as a transplant containing a population of engineered immune cells, e.g. T-cells. The transplanted cells administered to a recipient may be, e.g. autologous, syngeneic, or allogeneic cells.

As used herein, a “donor” is a mammal (e.g. a human) from which one or more cells are isolated prior to administration of the cells, or progeny thereof, into a recipient. The one or more cells may be, e.g. a population of immune cells or hematopoietic stem cells to be engineered, expanded, enriched, or maintained according to the methods described herewith prior to administration of the cells or the progeny thereof into a recipient. In the allogeneic setting contemplated herewith, a “donor” is not the patient to be treated.

“Expansion” in the context of cells refers to the increase in the number of a characteristic cell type, or cell types, from an initial cell population of cells, which may or may not be identical. The initial cells used for expansion may not be the same as the cells generated from expansion.

“Cell population” includes eukaryotic cells, such as mammalian, e.g. human, cells isolated from biological sources, for example, blood product or tissues. A cell population can derive from more than one cell.

As used herein, the term “pharmaceutical composition” refers to the active ingredient in combination with a pharmaceutically acceptable carrier and/or excipient e.g. a carrier and/or excipient commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of mammals, such as human beings, without excessive toxicity, irritation, allergic response, or other problem or complication, commensurate with a reasonable benefit/risk ratio.

As used herein, the term “administering,” refers to the placement of a compound, cell, or population of cells as disclosed herein into a subject by a method or route which results in at least partial delivery of the agent at a desired site. Pharmaceutical compositions comprising the compounds or cells disclosed herein can be administered by any appropriate route which results in an effective treatment in the patient. The patient who can be treated with the materials and methods disclosed herewith can be a mammal, including a human and a non-human primate.

As used herein, “nucleic acid” or “polynucleotides” refers to nucleotides and/or polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g. enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Nucleic acids can be either single stranded or double stranded.

The terms “polypeptide,” “peptide” and “protein” are used interchangeably to refer to a polymer of amino acid residues. The term also applies to amino acid polymers in which one or more amino acids are chemical analogues or modified derivatives of corresponding naturally-occurring amino acids.

As used herein, the terms “treat,” “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal (e.g. a human), and includes: (a) preventing the disease from occurring in a subject which may be predisposed to the disease but has not yet been diagnosed as having it; (b) inhibiting the disease, i.e., arresting its development; and (c) relieving the disease, e.g. causing regression of the disease, e.g. to completely or partially remove symptoms of the disease.

The term “subject” or “patient” as used herein includes mammals including non-human primates and humans.

An “effective amount” or “therapeutically effective amount” refers to that amount of a composition described herein which, when administered to a subject (e.g. human), is sufficient to aid in treating a disease. The amount of a composition that constitutes a “therapeutically effective amount” will vary depending on the cell preparations, the condition and its severity, the manner of administration, and the age of the subject to be treated, but can be determined routinely by one of ordinary skill in the art having regard to his own knowledge and to this disclosure. When referring to an individual active ingredient or composition, administered alone, a therapeutically effective dose refers to that ingredient or composition alone. When referring to a combination, a therapeutically effective dose refers to combined amounts of the active ingredients, compositions or both that result in the therapeutic effect, whether administered concurrently, simultaneously, or sequentially.

By “vector” is meant a nucleic acid molecule capable of transporting another nucleic acid to which it has been linked. A “vector” can include, but is not limited to, a viral vector, a plasmid, an oligonucleotide, a RNA vector or a linear or circular DNA or RNA molecule which may consist of a chromosomal, non-chromosomal, semisynthetic or synthetic nucleic acids. Preferred vectors are those capable of autonomous replication (episomal vector) and/or expression of nucleic acids to which they are linked (expression vectors). Large numbers of suitable vectors are known to those of skill in the art and commercially available. Viral vectors include retrovirus, adenovirus, parvovirus (e.g. adenoassociated viruses (AAV), coronavirus, negative strand RNA viruses such as orthomyxovirus (e.g. influenza virus), rhabdovirus (e.g. rabies and vesicular stomatitis virus), paramyxovirus (e.g. measles and Sendai), positive strand RNA viruses such as picornavirus and alphavirus, and double-stranded DNA viruses including adenovirus, herpesvirus (e.g. Herpes Simplex virus types 1 and 2, Epstein-Barr virus, cytomegalovirus), and poxvirus (e.g. vaccinia, fowlpox and canarypox). Other viruses include Norwalk virus, togavirus, flavivirus, reoviruses, papovavirus, hepadnavirus, and hepatitis virus, for example. Examples of retroviruses include avian leukosis-sarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields, et al., Eds., Lippincott-Raven Publishers, Philadelphia, 1996).

As used herein, the term “locus” is the specific physical location of a DNA sequence (e.g. of a gene) into a genome. The term “locus” can refer to the specific physical location of a rare-cutting endonuclease target sequence on a chromosome. Such a locus can comprise a target sequence that is recognized and/or cleaved by a sequence-specific endonuclease as described herein. It is understood that the locus of interest can not only qualify a nucleic acid sequence that exists in the main body of genetic material (i.e. in a chromosome) of a cell but also a portion of genetic material that can exist independently to said main body of genetic material such as plasmids, episomes, virus, transposons or in organelles such as mitochondria as non-limiting examples.

As used herewith, a nucleic acid sequence is said to be “placed under the transcriptional control of a promoter” if said nucleic acid sequence follows, or is at the 3′ end, of said promoter in such a manner that it is operably linked to said promoter and its transcription is controlled by said promoter.

The term “cleavage” when used in reference to nucleic acid refers to the breakage of the covalent backbone of a polynucleotide. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. Double stranded DNA, RNA, or DNA RNA hybrid cleavage can result in the production of either blunt ends or staggered ends.

“Sequence identity” refers to the identity between two nucleic acid molecules or polypeptides. It refers to the residues in the two sequences which are the same when the sequences are aligned for maximum correspondence. When a position in the compared sequence is occupied by the same base (or amino acid), then the molecules are identical at that position. A degree of identity between nucleic acid sequences (or amino acid sequences) is a function of the number of identical or matching nucleotides (or amino acids) at positions shared by the aligned nucleic acid sequences (or amino acid sequences). Various alignment algorithms and/or programs may be used to calculate the identity between two sequences, including FASTA, or BLAST which are available as a part of the GCG sequence analysis package (University of Wisconsin, Madison, Wis.), and can be used with, e.g. default setting. For example, polypeptides having at least 70%, 85%, 90%, 95%, 98% or 99% identity to specific polypeptides described herein and exhibiting substantially the same functions, as well as polynucleotide encoding such polypeptides, are contemplated.

“Fibroblast activation Protein” (“FAP”) is also generally called Prolyl endopeptidase FAP, or Fibroblast Activation Protein alpha (NCBI Gene ID: 2191). In some cases, a FAP polypeptide can be a human FAP polypeptide. Examples of FAP polypeptides that can be targeted by a FAP-CAR described herein include, without limitation, a human FAP polypeptide having the amino acid sequence set forth in NCBI Reference Sequence: NP_004451.2.

In one aspect, this document provides an engineered cell comprising:
a) an exogenous nucleic acid sequence encoding a chimeric antigen receptor (CAR) targeting the Fibroblast activation Protein (FAP) (“FAP-CAR”) placed under the transcriptional control of an exogenous or endogenous constitutive promoter; and
b) an exogenous nucleic acid sequence encoding a chimeric antigen receptor (CAR) targeting a tumor antigen (“tumor-CAR”) placed under the transcriptional control of an exogenous or endogenous inducible promoter;
wherein said exogenous nucleic acid sequences a) and b) are integrated in the cell's genome, and wherein the expression of the tumor-CAR is inducible upon activation of the cell.

The engineered cell can be any appropriate cell. For example, the cell containing exogenous nucleic acid sequences a) and b) can be an immune cell such as a T-cell, a NK-cell, or a macrophage. In some cases, the cell containing exogenous nucleic acid sequences a) and b) can be an iPSC that may be an intermediate in the production of an engineered immune cell, such as a T-cell, a NK-cell, or a macrophage, as described herein.

In some cases, an engineered cell provided herein can be a T-cell that has been genetically modified to suppress or repress expression of T-cell receptors (TCRs) (e.g. endogenous TCRs) at the T-cell surface and, optionally, to suppress or repress expression of at least one gene controlling MHC complex surface presentation such as a B2M gene that encodes a β2m polypeptide and/or a CIITA gene that encodes a CIITA polypeptide, and optionally to suppress or repress expression of a gene encoding a CD52 polypeptide, at the T-cell surface.

In some cases, a β2m polypeptide can be a human β2m polypeptide. Examples of B2M genes encoding β2m polypeptides which expression can be suppressed or repressed as described herein include, without limitation, a human B2M gene (e.g. NCBI Gene ID 567) encoding a β2m polypeptide having the amino acid sequence set forth in GeneBank Accession No. AAA51811.

In some cases, a CIITA polypeptide can be a human CIITA polypeptide. Examples of CIITA genes encoding CIITA polypeptides which expression can be suppressed or repressed as described herein include, without limitation, a human CIITA gene (e.g. NCBI Gene ID 4261) encoding a CIITA polypeptide having the amino acid sequence set forth in GeneBank Accession No. P33076.3 or No. AAU06586.

In some cases, a CD52 polypeptide can be a human CD52 polypeptide. Examples of CD52 genes (e.g. NCBI Gene ID 1043) encoding CD52 polypeptides which expression can be suppressed or repressed as described herein include, without limitation, a human CD52 polypeptide having the amino acid sequence set forth in GeneBank Accession No. AJC19276.

In some cases, an engineered T-cell described herein can be designed such that the CD52 gene, the B2M gene, or both the CD52 gene and the B2M gene are inactivated.

In some cases, an engineered T-cell described herein can be designed (e.g., genetically modified) to suppress or repress the expression of at least one gene encoding an immune checkpoint protein or receptor thereof. For example, an engineered T-cell described herein can be designed such that the programmed cell death 1 (PDCD1) gene, the CTLA4 gene, or both the PDCD1 gene and the cytotoxic T-lymphocyte associated protein 4 (CTLA4) gene are inactivated.

In some cases, a PD1 polypeptide can be a human PD1 polypeptide. Examples of PDCD1 genes encoding PD1 polypeptides which expression can be suppressed or repressed as described herein include, without limitation, a human PDCD1 gene (e.g. NCBI Gene ID 5133 or ENSG00000188389) encoding a polypeptide having the amino acid sequence set forth in GeneBank Accession No. UMM61402.1.

In some cases, a CTLA4 polypeptide can be a human CTLA4 polypeptide. Examples of CTLA4 genes encoding CTLA4 polypeptides which expression can be suppressed or repressed as described herein include, without limitation, a human CTLA4 gene (e.g. NCBI Gene ID 1493) encoding a CTLA4 polypeptide having the amino acid sequence set forth in GeneBank Accession No. AAL07473.

In some cases, an engineered T-cell described herein can be designed such that at least one gene encoding a TCR component, and a PDCD1 gene are inactivated.

In another aspect, this document provides a pharmaceutical composition comprising engineered immune cells (e.g., engineered T-cells), comprising: a) an exogenous nucleic acid sequence encoding a FAP-CAR placed under the transcriptional control of an exogenous or endogenous constitutive promoter; and b) an exogenous nucleic acid sequence encoding a tumor-CAR placed under the transcriptional control of an exogenous or endogenous inducible promoter; wherein said exogenous nucleic acid sequences a) and b) are integrated in the cell's genome, and wherein the expression of the tumor-CAR is inducible upon activation of the immune cell.

In some cases, a pharmaceutical composition described herein can be for use in the treatment of a cancer characterized by the presence of FAP in the tumor microenvironment.

In another aspect, this document provides methods for treating a cancer characterized by the presence of FAP in the tumor microenvironment, comprising administering, to a patient in need thereof, a therapeutically effective amount of engineered immune cells (e.g., engineered T-cells), comprising: a) an exogenous nucleic acid sequence encoding a FAP-CAR placed under the transcriptional control of an exogenous or endogenous constitutive promoter; and b) an exogenous nucleic acid sequence encoding a tumor-CAR placed under the transcriptional control of an exogenous or endogenous inducible promoter; wherein said exogenous nucleic acid sequences a) and b) are integrated in the cell's genome, and wherein the expression of the tumor-CAR is inducible upon activation of said immune cell.

The engineered cells and methods described herein can be part of an autologous or part of an allogenic treatment. By autologous, it is meant that cells used for treating patients are originating from said patient. By allogeneic, it is meant that the cells or population of cells used for treating patients are not originating from said patient but from a donor or from a cell line.

In some cases, engineered cells described herein can be administered to patients (e.g. humans) undergoing an immunosuppressive treatment. In some cases, the administered cells can be cells that were made resistant to at least one immunosuppressive agent. In some cases, the immunosuppressive treatment can help the selection and expansion of the engineered immune cells (e.g. engineered T-cells) within the patient.

Any appropriate route of administration can be used to administer cells described herein to a patient, including by aerosol inhalation, injection, ingestion, transfusion, implantation, and/or transplantation. The compositions described herein can be administered to a patient subcutaneously, intradermally, intratumorally, intranodally, intramedullary, intramuscularly, by intravenous or intralymphatic injection, or intraperitoneally. In some cases, a cell composition described herein can be administered to patient by intravenous injection, where the cells are capable of migrating to their desired site of action.

While individual needs vary, determination of optimal ranges of effective amounts of a given cell type for a particular disease or conditions within the skill of the art. An effective amount means an amount which provides a therapeutic or prophylactic benefit. The dosage administered will be dependent upon the age, health and weight of the recipient, kind of concurrent treatment, if any, frequency of treatment and the nature of the effect desired. In some cases, the administration of the cells or population of cells can comprise administration of about 10⁴-10⁹ cells per kg body weight. In some cases, about 10⁵ to 10⁶ cells/kg body weight, or about 10⁵ to 5×10⁶ cells/kg body weight, can be administered. All integer values of cell numbers within those ranges are contemplated.

The cells can be administered in one or more doses. In some cases, an effective amount of cells can be administered as a single dose. In some cases, an effective amount of cells can be administered as more than one dose over a period of time. Timing of administration is within the judgment of managing physician and depends on the clinical condition of the patient.

In some cases, administering engineered immune cells (e.g. T-cells), can include treating the patient with a myeloablative and/or immune suppressive regimen to deplete host bone marrow stem cells and prevent rejection. In some cases, the patient can be administered chemotherapy and/or radiation therapy. In some cases, the patient can be administered a reduced dose chemotherapy regimen. In some cases, reduced dose chemotherapy regimen with busulfan at 25% of standard dose can be sufficient to achieve significant engraftment of modified cells while reducing conditioning-related toxicity (Aiuti A. et al. (2013), Science 23; 341 (6148)). A stronger chemotherapy regimen can be based on administration of both busulfan and fludarabine as depleting agents for endogenous HSC. In some cases, the dose of busulfan and fludarabine can be approximately 50% and 30% of the ones employed in standard allogeneic transplantation. In some cases, the cells can be administered following B-cell ablative therapy such as agents that react with CD20, e.g. Rituxan. In some cases, the patient can be administered chemotherapy agents such as fludarabine, external-beam radiation therapy (XRT), cyclophosphamide, or antibodies such as OKT3 directed against CD3 or Alemtuzumab (Campath®, Lemtrada®) directed against CD52. In some cases, the patient can be administered with fludarabine and cyclophosphamide, and, optionally, Alemtuzumab.

In certain cases, the engineered immune cells (e.g. T-cells), can be administered to the subject as combination therapy comprising immunosuppressive agents. Exemplary immunosuppressive agents include sirolimus, tacrolimus, cyclosporine, mycophenolate, anti-thymocyte globulin, corticosteroids, calcineurin inhibitor, anti-metabolite, such as methotrexate, post-transplant cyclophosphamide or any combination thereof. In some cases, the subject can be pretreated with only sirolimus or tacrolimus as prophylaxis against GVHD. In some cases, the cells can be administered to the subject before an immunosuppressive agent. In some cases, the cells can be administered to the subject after an immunosuppressive agent. In some cases, the cells can be administered to the subject concurrently with an immunosuppressive agent. In some cases, the cells can be administered to the subject without an immunosuppressive agent. In some cases, the patient receiving genetically modified cells can receive immunosuppressive agent for less than 6 months, 5 months, 4 months, 3 months, 2 months, 1 month, 3 weeks, 2 weeks, or 1 week.

### 1. Engineered Immune Cells Comprising a FAP-CAR and a Tumor-CAR

The engineered cells expressing (i) a chimeric antigen receptor directed against Fibroblast Activation Protein (“FAP-CAR”) which expression is constitutive, and (ii) a chimeric antigen receptor directed against a tumor antigen (“tumor-CAR”) which expression is inducible upon activation of said cells, are not particularly limiting.

### 1.1. Type of Cells

The engineered cells described herewith can be immune cells, including T-cells, NK-cells, and macrophages.

The engineered cells described herewith can also be Induced Pluripotent Stem Cells (“iPSCs”), which could subsequently be differentiated into immune cells as described herewith. The engineered iPSCs as described herewith would, thus, be an intermediate product in the production of engineered immune cells according to the present disclosure.

In some cases, the engineered cells described herewith could be any differentiated cells, which could subsequently be de-differentiated into iPSCs, which in turn could subsequently be differentiated into immune cells as described herewith. The engineered differentiated cells as described herewith would thus be an intermediate product in the production of engineered immune cells according to the present disclosure. The genetic engineering as described herewith could be carried out on the differentiated cells, on the de-differentiated cells, or on the iPSCs.

Methods to produce iPSCs from differentiated cells are well known to the skilled person, they include methods based on nuclear transfer, usage of cell extracts and synthetic molecules, forced expression of defined genes and cytoplasmatic level modifications (Telpalo-Carpio et al (2013) J Stem Cells Regen Med. 9 (1): 2-8). Methods to produce immune cells from iPSCs are also well known to the skilled person, they include for instance the use of a serum- and feeder-free in vitro protocol of differentiation into T-cells as disclosed in Themeli et al. (Nature Biotechnology (2013) 31:928-933) and the protocol of differentiation into NK-cells under a completely chemically-defined condition as described in Matsubara et al. (Biochem Biophys Res Commun. (2019) 515 (1): 1-8).

Thus, one aspect relates to an engineered immune cell comprising:
a) an exogenous nucleic acid sequence encoding a chimeric antigen receptor (CAR) targeting a Fibroblast Activation Protein (FAP) (“FAP-CAR”) placed under the transcriptional control of an exogenous or endogenous constitutive promoter; and
b) an exogenous nucleic acid sequence encoding a chimeric antigen receptor (CAR) targeting a tumor antigen (“tumor-CAR”) placed under the transcriptional control of an exogenous or endogenous inducible promoter;
wherein said exogenous nucleic acid sequences a) and b) are integrated in the cell's genome; and
wherein the expression of the tumor-CAR is inducible upon activation of said immune cell.

In some cases, said immune cell can be a T-cell.

In some cases, said immune cell can be a NK-cell.

In some cases, said immune cell can be a macrophage.

Another aspect relates to an engineered iPSC comprising:
a) an exogenous nucleic acid sequence encoding a chimeric antigen receptor (CAR) targeting a Fibroblast Activation Protein (FAP) (“FAP-CAR”) placed under the transcriptional control of an exogenous or endogenous constitutive promoter; and
b) an exogenous nucleic acid sequence encoding a chimeric antigen receptor (CAR) targeting a tumor antigen (“tumor-CAR”) placed under the transcriptional control of an exogenous or endogenous inducible promoter;
wherein said exogenous nucleic acid sequences a) and b) are integrated in the cell's genome, and
wherein the expression of the tumor-CAR is inducible upon activation of the iPSC, or upon activation of the immune cell into which said engineered iPSC can further be differentiated.

In some cases, the engineered iPSC described herewith can be an intermediate product in the production of an engineered immune cell comprising said FAP-CAR and said tumor-CAR.

### 1.2. FAP-CAR and Tumor-CAR

By “chimeric antigen receptor” or “CAR” is generally meant a synthetic receptor comprising a targeting moiety that is associated with one or more signaling domains in a single fusion molecule. As defined herein, the term “chimeric antigen receptor” covers single chain CARs as well as multi-chain CARs. In some cases, the binding moiety of a CAR can comprise an antigen-binding domain of a single-chain antibody (scFv), comprising light chain and heavy chain variable fragments of a monoclonal antibody joined by a flexible linker. Binding moieties based on receptor or ligand domains have also been used successfully. The signaling domains for first generation CARs are derived from the cytoplasmic region of the CD3zeta or the Fc receptor gamma chains. First generation CARs have been shown to successfully redirect T-cell cytotoxicity. However, they failed to provide prolonged expansion and anti-tumor activity in vivo. Signaling domains from co-stimulatory molecules including CD28, OX-40 (CD134), and 4-1BB (CD137) have been added alone (second generation) or in combination (third generation) to enhance survival and increase proliferation of CAR modified T-cells. CARs are not necessarily only single chain polypeptides, as multi-chain CARs are also possible. According to the multi-chain CAR architecture, for instance as described in WO 2014/039523, the signalling domains and co-stimulatory domains are located on different polypeptide chains. Such multi-chain CARs can be derived from FcεRI, by replacing the high affinity IgE binding domain of FcεRI alpha chain by an extracellular ligand-binding domain such as scFv, whereas the N- and/or C-termini tails of FcεRI beta and/or gamma chains are fused to signal transducing domains and co-stimulatory domains, respectively. The extracellular ligand binding domain has the role of redirecting the immune cell (e.g. T-cell) specificity towards cell targets, while the signal transducing domains activate the immune cell response. CARs are generally expressed in effector immune cells to redirect their immune activity against antigens expressed at the surface of tumor cells from various malignancies including lymphomas and solid tumors. A component of a CAR is any functional subunit of a CAR that is encoded by an exogenous polynucleotide sequence introduced into the cell. For instance, this component can help the interaction with the target antigen, the stability or the localization of the CAR into the cell.

While the CARs of the present disclosure useful in the methods herein are not limited to a specific CAR structure, a nucleic acid that can be used to engineer the immune cells generally encodes a CAR comprising: an extracellular antigen-binding domain that binds to a tumor antigen or FAP (depending on the target of the CAR), a hinge, a transmembrane domain, and an intracellular domain comprising a stimulatory domain and/or a primary signalling domain. Generally, the extracellular antigen-binding domain is a scFv comprising a Heavy variable chain (VH) and a Light variable chain (VL) of an antibody binding to a specific antigen (e.g. to a tumor antigen or FAP) connected via a Linker. The transmembrane domain can be, for example, a CD8α transmembrane domain, a CD28 transmembrane domain, or a 4-1BB transmembrane domain. The co-stimulatory domain can be, for example, the 4-1BB co-stimulatory domain or CD28 co-stimulatory domain. The primary signalling domain can be, for example, the CD32 signalling domain.

The CARs as described herewith also generally comprise a signal peptide to direct the nascent protein to the endoplasmic reticulum and subsequent expression at the engineered cell's surface. The signal peptide is cleaved after addressing the CAR to the cell surface. The signal peptide comprised in the CARs described herewith can be a CD8α signal peptide, such as one having an amino acid sequence having at least 80%, at least 90%, at least 95%, at least 99% identity with SEQ ID NO: 84, or having an amino acid sequence having at least 80%, at least 90%, at least 95%, at least 99% identity with an alternative signal peptide of SEQ ID NO: 85.

TABLE 1
Sequence of different domains typically present in a CAR

| Functional | | |
|---|---|---|
| domains | SEQ ID # | amino acid sequence |
| CD8α signal | SEQ ID NO: 84 | MALPVTALLLPLALLLHAARP |
| peptide (or | | |
| sequence leader) | | |
| Alternative | SEQ ID NO: 85 | METDTLLLWVLLLWVPGSTG |
| signal peptide | | |
| FcγRIIIα hinge | SEQ ID NO: 86 | GLAVSTISSFFPPGYQ |
| CD8α hinge | SEQ ID NO: 87 | TTTPAPRPPTPAPTIASQPLSLRPEACRPAAGG |
| | | AVHTRGLDFACD |
| IgG1 hinge | SEQ ID NO: 88 | EPKSPDKTHTCPPCPAPPVAGPSVFLFPPKPKD |
| | | TLMIARTPEVTCVVVDVSHEDPEVKFNWYVDGV |
| | | EVHNAKTKPREEQYNSTYRVVSVLTVLHQDWLN |
| | | GKEYKCKVSNKALPAPIEKTISKAKGQPREPQV |
| | | YTLPPSRDELTKNQVSLTCLVKGFYPSDIAVEW |
| | | ESNGQPENNYKTTPPVLDSDGSFFLYSKLTVDK |
| | | SRWQQGNVFSCSVMHEALHNHYTQKSLSLSPGK |
| CD8α | SEQ ID NO: 89 | IYIWAPLAGTCGVLLLSLVITLYC |
| transmembrane | | |
| domain | | |
| CD28 | SEQ ID NO: 90 | FWVLVVVGGVLACYSLLVTVAFIIFWV |
| transmembrane | | |
| domain | | |
| 4-1BB | SEQ ID NO: 91 | IISFFLALTSTALLFLLFFLTLRFSVV |
| transmembrane | | |
| domain | | |
| 4-1BB co- | SEQ ID NO: 92 | KRGRKKLLYIFKQPFMRPVQTTQEEDGCSCRFP |
| stimulatory | | EEEEGGCEL |
| domain | | |
| CD28 co- | SEQ ID NO: 93 | RSKRSRLLHSDYMNMTPRRPGPTRKHYQPYAPP |
| stimulatory | | RDFAAYRS |
| domain | | |
| CD3ζ signalling | SEQ ID NO: 94 | RVKFSRSADAPAYQQGQNQLYNELNLGRREEYD |
| domain | | VLDKRRGRDPEMGGKPRRKNPQEGLYNELQKDK |
| | | MAEAYSEIGMKGERRRGKGHDGLYQGLSTATKD |
| | | TYDALHMQALPPR |
| Linker 1 | SEQ ID NO: 45 | GSTSGSGKPGSGEGSTK |
| Linker 2 | SEQ ID NO: 46 | GGGGSGGGGSGGGGS |

A FAP-CAR comprises an extracellular ligand (or antigen) binding domain that recognizes FAP. Hence, a FAP-CAR as described herewith comprises an extracellular FAP-binding domain.

A tumor-CAR comprises an extracellular ligand (or antigen) binding domain that recognizes a tumor antigen. Hence, a tumor-CAR as described herewith comprises a tumor antigen-binding domain.

The term “extracellular antigen binding domain” or “extracellular ligand binding domain” as used herein generally refers to an oligopeptide or polypeptide that is capable of binding a specific antigen, such as FAP or a tumor antigen. In some cases, the domain will be capable of interacting with a cell surface molecule, such as a ligand. For example, in some cases, an extracellular antigen-binding domain can be chosen to recognize an antigen that acts as a cell surface marker on target cells associated with a particular disease state. In some cases, said extracellular antigen-binding domain can comprise a single chain antibody fragment (scFv) comprising the heavy (VH) and the light (VL) variable fragment of a target-antigen-specific monoclonal antibody joined by a flexible linker. The antigen binding domain of a CAR expressed on the cell surface of the engineered cells described herein can be any domain that binds to the target antigen and that derives from, for example, a monoclonal antibody, a recombinant antibody, a human antibody, a humanized antibody, and a functional fragment thereof.

TABLE 2
Sequences of the VH and VL regions, and
corresponding ScFvs of some examples of
FAP-CARS

| VH/VL/ | | |
|---|---|---|
| ScFv | CAR | amino acid sequence |
| Heavy | CLSFAP1- | QVQLVQSGAEVKKPGASVKVSCKTSRYTFTEYTIH |
| variable | CAR | WVRQAPGQRLEWIGGINPNNGIPNYNQKFKGRVTI |
| region | | TVDTSASTAYMELSSLRSEDTAVYYCARRRIAYGY |
| | | DEGHAMDYWGQGTLVTVSS (SEQ ID NO: 7) |
| Light | | DIVMTQSPDSLAVSLGERATINCKSSQSLLYSRNQ |
| variable | | KNYLAWYQQKPGQPPKLLIFWASTRESGVPDRFSG |
| region | | SGFGTDFTLTISSLQAEDVAVYYCQQYFSYPLTFG |
| | | QGTKVEI (SEQ ID NO: 8) |
| ScFv | | SEQ ID NO: 9 |
| Heavy | CLSFAP2- | EVQLQQSGPELVKPGASVRMSCKASGYTFTDYYMK |
| variable | CAR | WVKQSLGKSLEWIGDIYPNNGEIPYNQKFKGKATL |
| region | | TADKTSSTAYMQLNSLTSEDSAVYYCVRGYYYGLA |
| | | MDYWGQGTSVTSVV (SEQ ID NO: 18) |
| Light | | QAVVTQESALTSPGETVTLTCRSSTGAVTTSNYAN |
| variable | | WVQEKPDRLFTGLIGATNNRAPGVPARFSGSLIGD |
| region | | KAALTITGAQTEDEAIYFCALWYSNHFIFGSGTKV |
| | | TVL (SEQ ID NO: 19) |
| ScFv | | SEQ ID NO: 20 |
| Heavy | CLSFAP3- | QVQLVQSGAEVKKPGASVKVSCKASGYTFTENIIH |
| variable | CAR | WVRQAPGQGLEWMGWFHPGSGSIKYNEKFKDRVTM |
| region | | TADTSTSTVYMELSSLRSEDTAVYYCARHGGTGRG |
| | | AMDYWGQGTLVTVSS (SEQ ID NO: 29) |
| Light | | DIQMTQSPSSLSASVGDRVTITCRASKSVSTSAYS |
| variable | | YMHWYQQKPGKAPKLLIYLASNLESGVPSRFSGSG |
| region | | SGTDFTLTISSLQPEDFATYYCQHSRELPYTFGQG |
| | | TKLEIKR (SEQ ID NO: 30) |
| ScFv | | SEQ ID NO: 31 |
| Heavy | CLSFAP4- | EVQLLESGGGLVQPGGSLRLSCAASGFTFSSYAMS |
| variable | CAR | WVRQAPGKGLEWVSAIIGSGASTYYADSVKGRFTI |
| region | | SRDNSKNTLYLQMNSLRAEDTAVYYCAKGWFGGFN |
| | | YWGQGTLVTVSS (SEQ ID NO: 40) |
| Light | | EIVLTQSPGTLSLSPGERATLSCRASQSVTSSYLA |
| variable | | WYQQKPGQAPRLLINVGSRRATGIPDRFSGSGSGT |
| region | | DFTLTISRLEPEDFAVYYCQQGIMLPPTFGQGTKV |
| | | EIK (SEQ ID NO: 41) |
| ScFv | | SEQ ID NO: 42 |

In some cases, said FAP-CAR comprises an extracellular FAP-binding-domain comprising:
a) the H-CDRs of SEQ ID NO: 1, SEQ ID NO: 2, and SEQ ID NO: 3, and the L-CDRs of SEQ ID NO: 4, SEQ ID NO: 5, and SEQ ID NO: 6, and optionally an amino acid sequence having at least 80%, at least 90%, at least 95%, or at least 99% identity with amino acid sequence SEQ ID NO: 9;
b) the H-CDRs of SEQ ID NO: 12, SEQ ID NO: 13, and SEQ ID NO: 14, and the L-CDRs of SEQ ID NO: 15, SEQ ID NO: 16, and SEQ ID NO: 17, and optionally an amino acid sequence having at least 80%, at least 90%, at least 95%, or at least 99% identity with amino acid sequence SEQ ID NO: 20;
c) the H-CDRs of SEQ ID NO: 23, SEQ ID NO: 24, and SEQ ID NO: 25, and the L-CDRs of SEQ ID NO: 26, SEQ ID NO: 27, and SEQ ID NO: 28, and optionally an amino acid sequence having at least 80%, at least 90%, at least 95%, or at least 99% identity with amino acid sequence SEQ ID NO: 31; or
d) the H-CDRs of SEQ ID NO: 34, SEQ ID NO: 35, and SEQ ID NO: 36, and the L-CDRs of SEQ ID NO: 37, SEQ ID NO: 38, and SEQ ID NO: 39, and optionally an amino acid sequence having at least 80%, at least 90%, at least 95%, or at least 99% identity with amino acid sequence SEQ ID NO: 42.

In some cases, the FAP-CAR comprises an extracellular FAP-binding-domain comprising a VH region comprising SEQ ID NO: 7 and a VL region comprising SEQ ID NO: 8. In some cases, the FAP-CAR comprises an extracellular FAP-binding-domain comprising an amino acid sequence having at least about 80%, 85%, 90%, 91%, 92%, 93%, 94%, 95%, 96%, 97%, 98%, or 99% identity to a VH region comprising SEQ ID NO: 7 and an amino acid sequence having at least about 80%, 85%, 90%, 91%, 92%, 93%, 94%, 95%, 96%, 97%, 98%, or 99% identity to a VL region comprising SEQ ID NO: 8. In some cases, the extracellular FAP-binding-domain comprises an amino acid sequence comprising complementarity determining regions (CDRs) comprised in SEQ ID NO: 7 and SEQ ID NO: 8. In some cases, the H-CDRs comprised in SEQ ID NO: 7 comprise amino acids sequences of SEQ ID NO: 1 to SEQ ID NO: 3. In some cases, the L-CDRs comprised in SEQ ID NO: 8 comprise amino acids sequences of SEQ ID NO: 4 to SEQ ID NO: 6. In some cases, the FAP-CAR comprises an extracellular FAP-binding-domain comprising (i) the CDRs comprised in SEQ ID NOs: 7 and 8, and (ii) an amino acid sequence having at least about 80%, 85%, 90%, 91%, 92%, 93%, 94%, 95%, 96%, 97%, 98%, or 99% identity to a VH region comprising SEQ ID NO: 7, and (iii) an amino acid sequence having at least about 80%, 85%, 90%, 91%, 92%, 93%, 94%, 95%, 96%, 97%, 98%, or 99% identity to a VL region comprising SEQ ID NO: 8.

In some cases, the FAP-CAR comprises an extracellular FAP-binding-domain comprising a VH region comprising SEQ ID NO: 18 and a VL region comprising SEQ ID NO: 19. In some cases, the FAP-CAR comprises an extracellular FAP-binding-domain comprising an amino acid sequence having at least about 80%, 85%, 90%, 91%, 92%, 93%, 94%, 95%, 96%, 97%, 98%, or 99% identity to a VH region comprising SEQ ID NO: 18 and an amino acid sequence having at least about 80%, 85%, 90%, 91%, 92%, 93%, 94%, 95%, 96%, 97%, 98%, or 99% identity to a VL region comprising SEQ ID NO: 19. In some cases, the extracellular FAP-binding-domain comprises an amino acid sequence comprising complementarity determining regions (CDRs) comprised in SEQ ID NO: 18 and SEQ ID NO: 19. In some cases, the H-CDRs comprised in SEQ ID NO: 18 comprise amino acids sequences of SEQ ID NO: 12 to SEQ ID NO: 14. In some cases, the L-CDRs comprised in SEQ ID NO: 19 comprise amino acids sequences of SEQ ID NO: 15 to SEQ ID NO: 17. In some cases, the FAP-CAR comprises an extracellular FAP-binding-domain comprising (i) the CDRs comprised in SEQ ID NOs: 18 and 19, and (ii) an amino acid sequence having at least about 80%, 85%, 90%, 91%, 92%, 93%, 94%, 95%, 96%, 97%, 98%, or 99% identity to a VH region comprising SEQ ID NO: 18, and (iii) an amino acid sequence having at least about 80%, 85%, 90%, 91%, 92%, 93%, 94%, 95%, 96%, 97%, 98%, or 99% identity to a VL region comprising SEQ ID NO: 19.

In some cases, the FAP-CAR comprises an extracellular FAP-binding-domain comprising a VH region comprising SEQ ID NO: 29 and a VL comprising SEQ ID NO: 30. In some cases, the FAP-CAR comprises an extracellular FAP-binding-domain comprising an amino acid sequence having at least about 80%, 85%, 90%, 91%, 92%, 93%, 94%, 95%, 96%, 97%, 98%, or 99% identity to a VH region comprising SEQ ID NO: 29 and an amino acid sequence having at least about 80%, 85%, 90%, 91%, 92%, 93%, 94%, 95%, 96%, 97%, 98%, or 99% identity to a VL region comprising SEQ ID NO: 30. In some cases, the extracellular FAP-binding-domain comprises an amino acid sequence comprising complementarity determining regions (CDRs) comprised in SEQ ID NO: 29 and SEQ ID NO: 30. In some cases, the CDRs comprised in SEQ ID NO: 29 comprise amino acids sequences of SEQ ID NO: 23 to SEQ ID NO: 25. In some cases, the CDRs comprised in SEQ ID NO: 30 comprise amino acids sequences of SEQ ID NO: 26 to SEQ ID NO: 28. In some cases, the FAP-CAR comprises an extracellular FAP-binding-domain comprising (i) the CDRs comprised in SEQ ID NOs: 29 and 30, and (ii) comprising an amino acid sequence having at least about 80%, 85%, 90%, 91%, 92%, 93%, 94%, 95%, 96%, 97%, 98%, or 99% identity to a VH region comprising SEQ ID NO: 29, and (iii) an amino acid sequence having at least about 80%, 85%, 90%, 91%, 92%, 93%, 94%, 95%, 96%, 97%, 98%, or 99% identity to a VL region comprising SEQ ID NO: 30.

In some cases, the FAP-CAR comprises an extracellular FAP-binding-domain comprising a VH region comprising SEQ ID NO: 40 and a VL comprising SEQ ID NO: 41. In some cases, the FAP-CAR comprises an extracellular FAP-binding-domain comprising an amino acid sequence having at least about 80%, 85%, 90%, 91%, 92%, 93%, 94%, 95%, 96%, 97%, 98%, or 99% identity to a VH region comprising SEQ ID NO: 40 and an amino acid sequence having at least about 80%, 85%, 90%, 91%, 92%, 93%, 94%, 95%, 96%, 97%, 98%, or 99% identity to a VL region comprising SEQ ID NO: 41 In some cases, the extracellular FAP-binding-domain comprises an amino acid sequence comprising complementarity determining regions (CDRs) comprised in SEQ ID NO: 40 and SEQ ID NO: 41. In some cases, the CDRs comprised in SEQ ID NO: 40 comprise amino acids sequences of SEQ ID NO: 34 to SEQ ID NO: 36. In some cases, the CDRs comprised in SEQ ID NO: 41 comprise amino acids sequences of SEQ ID NO: 37 to SEQ ID NO: 39. In some cases, the FAP-CAR comprises an extracellular FAP-binding-domain comprising (i) the CDRs comprised in SEQ ID NOs: 40 and 41, and (ii) comprising an amino acid sequence having at least about 80%, 85%, 90%, 91%, 92%, 93%, 94%, 95%, 96%, 97%, 98%, or 99% identity to a VH region comprising SEQ ID NO: 40, and (iii) an amino acid sequence having at least about 80%, 85%, 90%, 91%, 92%, 93%, 94%, 95%, 96%, 97%, 98%, or 99% identity to a VL region comprising SEQ ID NO: 41.

TABLE 3
Sequences of the CDRs comprised in the ScFvs of
some examples of FAP-CARS

| Chain | CDR1 | CDR2 | CDR3 |
|---|---|---|---|
| CLSFAP1- | YTFTEYTIH | GINPNNGIPNYNQKF | RRIAYGYDEGHAMDY |
| heavy chain | (SEQ ID NO: 1) | (SEQ ID NO: 2) | (SEQ ID NO: 3) |
| CLSFAP1- | QSLLYSRNQKNYLA | LLIFWASTRES | QQYFSYPLT |
| light chain | (SEQ ID NO: 4) | (SEQ ID NO: 5) | (SEQ ID NO: 6) |
| CLSFAP2- | YTFTDYYMK | DIYPNNGEIPYNQKF | VRGYYYGLAMDY |
| heavy chain | (SEQ ID NO: 12) | (SEQ ID NO: 13) | (SEQ ID NO: 14) |
| CLSFAP2- | TGAVTTSNYAN | GLIGATNNRAP | ALWYSNHFI |
| light chain | (SEQ ID NO: 15) | (SEQ ID NO: 16) | (SEQ ID NO: 17) |
| CLSFAP3- | YTFTENIIH | WFHPGSGSIKYNEKF | HGGTGRGAMDY |
| heavy chain | (SEQ ID NO: 23) | (SEQ ID NO: 24) | (SEQ ID NO: 25) |
| CLSFAP3- | KSVSTSAYSYMH | LLIYLASNLES | QHSRELPYT |
| light chain | (SEQ ID NO: 26) | (SEQ ID NO: 27) | (SEQ ID NO: 28) |
| CLSFAP4- | FTFSSYAMS | VSAIIGSGASTYYAD | KGWFGGFNY |
| heavy chain | (SEQ ID NO: 34) | SV (SEQ ID NO: | (SEQ ID NO: 36) |
| | | 35) | |
| CLSFAP4- | QSVTSSYLA | LLINVGSRRATGI | QQGIMLPPT |
| light chain | (SEQ ID NO: 37) | (SEQ ID NO: 38) | (SEQ ID NO: 39) |

In some cases, the amino acid sequence comprising a VH region and the amino acid sequence comprising a VL region are separated by one or more linker amino acid residues. The number of amino acids constituting the linker is not necessarily limiting, but in some cases the linker is at least about 5 amino acids in length, such as at least about 10 amino acids in length. In some cases, the linker is between about 10-25 amino acids in length. In some cases, the linker sequence is selected from any one of SEQ ID NOs: 45-46.

In some cases, the extracellular FAP-binding-domain comprising the VH region and the VL region from a monoclonal anti-FAP antibody can comprise a sequence selected from any one of SEQ ID NO: 9, SEQ ID NO: 20, SEQ ID NO: 31 and SEQ ID NO: 42. In some cases, the extracellular FAP-binding-domain can comprise an amino acid sequence having at least about 80%, 85%, 90%, 91%, 92%, 93%, 94%, 95%, 96%, 97%, 98%, or 99% identity to any one of SEQ ID NO: 9, SEQ ID NO: 20, SEQ ID NO: 31 and SEQ ID NO: 42, and, optionally, the CDRs of SEQ ID NO: 1 to 6, SEQ ID NO: 12 to 17, SEQ ID NO: 23 to 28, and SEQ ID NO: 34 to 39, respectively.

In some cases, the FAP-CAR comprises an extracellular ligand binding domain that recognizes FAP, a transmembrane domain, and one or more intracellular signalling domains. In some cases, the FAP-CAR comprises a hinge region that separates the extracellular ligand binding domain and the transmembrane domains.

In some cases, the FAP-CAR comprises:
(a) an extracellular FAP-binding-domain comprising VH and VL from a monoclonal anti-FAP antibody,
(b) a hinge selected from a FcγRIII hinge, a CD8α hinge and an IgG1 hinge,
(c) a transmembrane domain selected from a CD8α transmembrane domain and a CD28 transmembrane domain, and
(d) a cytoplasmic domain including a CD3 zeta signalling domain and, optionally, a co-stimulatory domain from 4-1BB or CD28.

In some cases, the FAP-CAR comprises a CD8α hinge.

In some cases, said FAP-CAR comprises a CD8α hinge, a CD8α transmembrane domain, and optionally a co-stimulatory domain from 4-1BB.

In some cases, said FAP-CAR comprises a CD8α hinge, a CD28 transmembrane domain, and optionally a co-stimulatory domain from CD28.

In some cases, the engineered cells as described herewith comprise a FAP-CAR without a co-stimulatory domain (e.g. without the costimulatory domain from 4-1BB or from CD28) and a tumor-CAR comprising a co-stimulatory domain (e.g. a co-stimulatory domain from 4-1BB or from CD28).

In some cases, the FAP-CAR comprises a co-stimulatory domain from CD28 and the tumor-CAR comprises a co-stimulatory domain from 4-1BB.

In some cases, the FAP-CAR can have an amino acid sequence selected from any one of SEQ ID NO: 10, SEQ ID NO: 11, SEQ ID NO: 21, SEQ ID NO: 22, SEQ ID NO: 32, SEQ ID NO: 33, SEQ ID NO: 43, SEQ ID NO: 44. In some cases, the FAP-CAR can comprise an amino acid sequence having at least about 80%, 85%, 90%, 91%, 92%, 93%, 94%, 95%, 96%, 97%, 98%, or 99% identity to any one of SEQ ID NO: 10, SEQ ID NO: 21, SEQ ID NO: 32, SEQ ID NO: 43, and, optionally, the CDRs of SEQ ID NO: 1 to 6, SEQ ID NO: 12 to 17, SEQ ID NO: 23 to 28, SEQ ID NO: 34 to 39, respectively.

In some cases, the nucleic acid sequence encoding the FAP-CAR described herewith comprises a nucleic acid sequence selected from any one of SEQ ID NO: 107, SEQ ID NO: 108, SEQ ID NO: 109, and SEQ ID NO: 110. In some cases, the nucleic acid sequence encoding the FAP-CAR described herewith comprises a nucleic acid sequence having at least about 80%, 85%, 90%, 91%, 92%, 93%, 94%, 95%, 96%, 97%, 98%, or 99% identity to any one of SEQ ID NO: 107, SEQ ID NO: 108, SEQ ID NO: 109, or SEQ ID NO: 110, and encodes a FAP-CAR comprising an amino acid sequence selected from any one of SEQ ID NO: 10, SEQ ID NO: 21, SEQ ID NO: 32, and SEQ ID NO: 43, respectively.

In some cases, the engineered immune cells (e.g. the engineered T cells) as described herewith for their use in allogeneic settings are endowed with FAP-CARs and tumor-CARs as described herewith comprising a co-stimulatory domain from CD28 in order to trigger a faster activation of said immune cells.

In some cases, the FAP-CAR can comprise an amino acid sequence having at least about 80%, 85%, 90%, 91%, 92%, 93%, 94%, 95%, 96%, 97%, 98%, or 99% identity to any one of SEQ ID NO: 11, SEQ ID NO: 22, SEQ ID NO: 33, SEQ ID NO: 44, comprising the CDRs of SEQ ID NO: 1 to 6, SEQ ID NO: 12 to 17, SEQ ID NO: 23 to 28, SEQ ID NO: 34 to 39, respectively.

As mentioned above, the engineered immune cell of the present disclosure also comprises an exogenous nucleic acid sequence encoding a chimeric antigen receptor (CAR) targeting a tumor antigen (“tumor-CAR”) placed under the transcriptional control of an exogenous or endogenous inducible promoter.

As used herewith the term “tumor antigen” is meant to cover “tumor-specific antigens”, “tumor associated antigens”. Tumor-Specific Antigens (TSA) are generally present only on tumor cells and not on any other cell, while Tumor-Associated Antigens (TAA) are present on some tumor cells and also present on some normal cells. “Tumor antigen,” as meant herein, also refers to mutated forms of a protein, which only appears in that form in tumors, while the non-mutated form is observed in non-tumoral tissues.

A tumor antigen can be an antigen specific of, or associated with, a solid tumor. The tumor antigen is not limiting. In some cases, the tumor antigen is selected from the group consisting of CEA, ERBB2, EGFR, GD2, mesothelin, MUC1, PSMA, GD2, PSMA1, LAP3, ANXA3, Tumor-associated glycoprotein 72 (TAG72), MUC16, 5T4, FRα, MUC28z, NKG2D, HRG1β, prostate stem cell antigen (PSCA), prostate-specific membrane antigen (PSMA), carboxy-anhydrase-IX (CA-IX), Trop2, claudin18.2, folate receptor 1 (FOLR1), CXCR2, B7-H3, CD133, CD24, receptor tyrosine kinase-like orphan receptor 1-specific (ROR1), EGFR, EGFRvIII, VEGF, erythropoietin-producing hepatocellular carcinoma A2 (EphA2), DLL3, glypican-3, epithelial cell adhesion molecule (EpCAM), GUCY2C (Guanylate Cyclase 2C), doublecortin-like kinase 1 (DCLK1), HER receptors HER1, HER2, HER3, HER4, PEM, A33, G250, carbohydrate antigens Le^{y}, Le^{x}, Le^{b}, STEAP1, CD166, CD24, CD44, E-cadherin, SPARC, and ERBB3. See, e.g. Marofi et al. Stem Cell Res Ther (2021) 12, 81, which is incorporated by reference herein.

In some cases, the tumor antigen is selected from the group consisting of mesothelin, Trop2, MUC1, EGFR, and VEGF. In some cases, the antigen is selected from the group consisting of Mesothelin, MUC1, and Trop2.

A tumor antigen can also be an antigen specific of, or associated with, a haematological cancer characterized by the presence of FAP in the tumor microenvironment such as myelofibrosis, myelodysplastic syndromes, acute myeloid leukemia, non-Hodgkin's lymphoma, multiple myeloma.

In some cases, the tumor antigen associated with an haematological cancer is selected from the group consisting of BCMA, CD19, CD20, CD22, CD30, CD123, CD70, CD33, CD135, CD44, CD276, CD2, CD3, CD4, CD5, CD7, CD8, CD10, CD37, CD79, CD79a, CD80, CD138, CD47, CRLF2, CD38, CLL-1, NKG2D, CALR, IL1RAP, ILT3, TIM3, CD96, VISTA, CS1, TACI, APRIL, GPRC5D, and CD44v6.

In some cases, the tumor antigen associated with an haematological cancer is selected from the group consisting of BCMA, CD19, CD123, CD20, CD22, CS1, CD138, CD80, CD2, CD3, CD4, CD5, CD7 and CD8.

In some cases, the tumor antigen associated with an haematological cancer is selected from the group consisting of BCMA, CD19, CD123, CD20, CD22, and CS1.

TABLE 4
Sequences of the CDRs comprised in the ScFvs
of some examples of tumor-CARS

| Chain | CDR1 | CDR2 | CDR3 |
|---|---|---|---|
| Trop2-heavy | YTFTNYGMN | MGWINTYTGEPTYT | GGFGSSYWYFDV |
| chain | (SEQ ID NO: | (SEQ ID NO: 48) | (SEQ ID NO: |
| | 47) | | 49) |
| Trop2-light | QDVSIAVA | LLIYSASYRYT | QQHYITPLT |
| chain | (SEQ ID NO: | (SEQ ID NO: 51) | (SEQ ID NO: |
| | 50) | | 52) |
| MUC1-heavy | NYGLS | ENHPGSGIIYHNEK | SSGTRGFAY |
| chain | (SEQ ID NO: | FR (SEQ ID NO: | (SEQ ID NO: |
| | 55) | 56) | 57) |
| MUC1-light | RSSQSIVHSNG | LLIYKVSNRFS | FQGSHGPWT |
| chain | NTYLE (SEQ | (SEQ ID NO: 59) | (SEQ ID NO: |
| | ID NO: 58) | | 60) |
| Mesothelin- | INNNNYYWT | WIGYIYYSGSTFYN | EDTMTGLDV |
| heavy chain | (SEQ ID NO: | PSLKS (SEQ ID | (SEQ ID NO: |
| | 63) | NO: 64) | 65) |
| Mesothelin- | QSINNYLN | LLIYAASSLQS | QQTYSNPT |
| light chain | (SEQ ID NO: | (SEQ ID NO: 67) | (SEQ ID NO: |
| | 66) | | 68) |

TABLE 5
Sequences of the ScFv of some examples of tumor-CARs

| SEQ | ScFv of tumor- | |
|---|---|---|
| ID: # | CAR | Amino acid sequence |
| 53 | Trop2_ ScFv | DIQLTQSPSSLSASVGDRVSITCKASQDVSIAVAWYQQ |
| | | KPGKAPKLLIYSASYRYTGVPDRFSGSGSGTDFTLTIS |
| | | SLQPEDFAVYYCQQHYITPLTFGAGTKVEIKGGGGSGG |
| | | GGSGGGGSQVQLQQSGSELKKPGASVKVSCKASGYTFT |
| | | NYGMNWVKQAPGQGLKWMGWINTYTGEPTYTDDFKGRF |
| | | AFSLDTSVSTAYLQISSLKADDTAVYFCARGGFGSSYW |
| | | YFDVWGQGSLVTVSS |
| 69 | Mesothelin_ScFv1 | QVQLQESGPGLVKPSQTLSLTCTVSGGSINNNNYYWTW |
| | | IRQHPGKGLEWIGYIYYSGSTFYNPSLKSRVTISVDTS |
| | | KTQFSLKLSSVTAADTAVYYCAREDTMTGLDVWGQGTT |
| | | VTVSSGGGGSGGGGSGGGGSDIQMTQSPSSLSASVGDR |
| | | VTITCRASQSINNYLNWYQQKPGKAPTLLIYAASSLQS |
| | | GVPSRFSGSRSGTDFTLTISSLQPEDFAAYFCQQTYSN |
| | | PTFGQGTKVEVK |
| 71 | Mesothelin_ScFv2 | QVQLQQPGAELVKPGASMKLSCKASGYTFTSYWMHWVK |
| | | QRPGQGLEWIGMIHPNSDNTIYYEKFKSKATLTVDKSS |
| | | STAYMQLSSLTSEDSAVYYCAIIITPVVPKFDYWGQGT |
| | | TLTVSSGGGGSGGGGSGGGGSDIVMTQSHQFMSTSVGD |
| | | RVSVTCKASHDVGTSVAWYQQKPGQSPKLLIYWASTRH |
| | | TGVPDRFTGSGSGTDFTLTISNVQSEDLADYFCQQYSS |
| | | YPLTFGAGTKLELKRA |
| 61 | MUC1 ScFv | MEWIWIFLFILSGTAGVQSQVQLQQSGAELARPGASVK |
| | | LSCKASGYTFTNYGLSWVKQRTGQGLEWIGENHPGSGI |
| | | IYHNEKFRGKATLTADKSSSTAYVQLSSLTSEDSAVYF |
| | | CARSSGTRGFAYWGQGTLVTVSAGGGGSGGGGSGGGGS |
| | | MKLPVRLLVLMFWIPASSSDVLMTQTPLSLPVSLGDQA |
| | | SISCRSSQSIVHSNGNTYLEWYLQKPGQSPKLLIYKVS |
| | | NRFSGVPDRFSGSGSGTDFTLKISRVEAEDLGVYYCFQ |
| | | GSHGPWTFGGGTKLEIKRA |

Similarly to the FAP-CAR described herewith, the tumor-CAR described herewith can comprise:
(a2) an extracellular ligand binding-domain comprising VH and VL amino acid sequences from a monoclonal antibody,
(b2) a hinge selected from a FcγRIII hinge, a CD8α hinge and an IgG1 hinge,
(c2) a transmembrane domain comprising a CD8α transmembrane domain or a CD28 transmembrane domain, and
(d2) a cytoplasmic domain comprising a CD3 zeta signaling domain and a co-stimulatory domain from 4-1BB or from CD28.

However, the tumor-CAR differs from the FAP-CAR by, at least, its extracellular ligand binding domain, since the extracellular ligand binding-domain of the tumor-CAR can comprise VH and VL amino acid sequences from a monoclonal anti-tumor antigen antibody, whereas the extracellular ligand binding-domain of a FAP-CAR can comprise VH and VL amino acid sequences from a monoclonal anti-FAP antibody.

In some cases, the tumor-CAR can comprise an extracellular binding-domain comprising:
a) the H-CDRs of SEQ ID NO: 47, SEQ ID NO: 48, and SEQ ID NO: 49, and the L-CDRs of SEQ ID NO: 50, SEQ ID NO: 51, and SEQ ID NO: 52, and optionally an amino acid sequence having at least 80%, at least 90%, at least 95%, or at least 99% identity with amino acid sequence SEQ ID NO: 53;
b) the H-CDRs of SEQ ID NO: 55, SEQ ID NO: 56, and SEQ ID NO: 57, and the L-CDRs of SEQ ID NO: 58, SEQ ID NO: 59, and SEQ ID NO: 60, and optionally an amino acid sequence having at least 80%, at least 90%, at least 95%, or at least 99% identity with amino acid sequence SEQ ID NO: 61;
c) the H-CDRs of SEQ ID NO: 63, SEQ ID NO: 64, and SEQ ID NO: 65, and the L-CDRs of SEQ ID NO: 66, SEQ ID NO: 67, and SEQ ID NO: 68, and optionally an amino acid sequence having at least 80%, at least 90%, at least 95%, or at least 99% identity with amino acid sequence SEQ ID NO: 69; or
d) the H-CDRs and the L-CDRs comprised in the amino acid sequence of SEQ ID NO: 71, and optionally an amino acid sequence having at least 80%, at least 90%, at least 95%, or at least 99% identity with amino acid sequence SEQ ID NO: 71.

In some cases, the tumor-CAR can be specific for Mesothelin (MESO-CAR) and can have an amino acid sequence of SEQ ID NO: 70. In some cases, the MESO-CAR can comprise an amino acid sequence having at least about 80%, 85%, 90%, 91%, 92%, 93%, 94%, 95%, 96%, 97%, 98%, or 99% identity to SEQ ID NO: 70, and, optionally, the CDRs of SEQ ID NO: 63 to SEQ ID NO: 68.

In some cases, the nucleic acid sequence encoding the MESO-CAR described herewith can comprise a nucleic acid sequence of SEQ ID NO: 104. In some cases, the nucleic acid sequence encoding the MESO-CAR described herewith can comprise a nucleic acid sequence having at least about 80%, 85%, 90%, 91%, 92%, 93%, 94%, 95%, 96%, 97%, 98%, or 99% identity to SEQ ID NO: 104, and can encode a MESO-CAR comprising the amino acid sequence of SEQ ID NO: 70.

In some cases, the tumor-CAR can be specific for Trop2 (Trop2-CAR) and can have an amino acid sequence of SEQ ID NO: 54. In some cases, the Trop2-CAR can comprise an amino acid sequence having at least about 80%, 85%, 90%, 91%, 92%, 93%, 94%, 95%, 96%, 97%, 98%, or 99% identity to SEQ ID NO: 54, and, optionally, the CDRs of SEQ ID NO: 47 to SEQ ID NO: 52.

In some cases, the nucleic acid sequence encoding the Trop2-CAR described herewith can comprise a nucleic acid sequence of SEQ ID NO: 106. In some cases, the nucleic acid sequence encoding the Trop2-CAR described herewith can comprise a nucleic acid sequence having at least about 80%, 85%, 90%, 91%, 92%, 93%, 94%, 95%, 96%, 97%, 98%, or 99% identity to SEQ ID NO: 106, and can encode a Trop2-CAR comprising the amino acid sequence of SEQ ID NO: 54.

In some cases, the tumor-CAR can be specific for Mucin1 (MUC1-CAR) and can have an amino acid sequence of SEQ ID NO: 62. In some cases, the MUC1-CAR can comprise an amino acid sequence having at least about 80%, 85%, 90%, 91%, 92%, 93%, 94%, 95%, 96%, 97%, 98%, or 99% identity to SEQ ID NO: 62, and, optionally, the CDRs of SEQ ID NO: 55 to SEQ ID NO: 60.

In some cases, the nucleic acid sequence encoding the MUC1-CAR described herewith can comprise a nucleic acid sequence of SEQ ID NO: 105. In some cases, the nucleic acid sequence encoding the MUC1-CAR described herewith can comprise a nucleic acid sequence having at least about 80%, 85%, 90%, 91%, 92%, 93%, 94%, 95%, 96%, 97%, 98%, or 99% identity to SEQ ID NO: 105, and can encode a MUC1-CAR comprising the amino acid sequence of SEQ ID NO: 62.

In some cases, the tumor-CAR can be specific for CS1 (CS1-CAR) and can have an amino acid sequence of SEQ ID NO: 96. In some cases, the CS1-CAR can comprise an amino acid sequence having at least about 80%, 85%, 90%, 91%, 92%, 93%, 94%, 95%, 96%, 97%, 98%, or 99% identity to SEQ ID NO: 96, and, optionally, the CDRs comprised in SEQ ID NO: 96.

In some cases, the tumor-CAR and the FAP-CAR not only differ by their extracellular ligand binding-domain but also by one or more of the following domains: the hinge, the transmembrane domain, and the co-stimulatory domain.

In some cases, the FAP-CAR comprises a co-stimulatory domain from CD28, while the tumor-CAR comprises a costimulatory domain from 4-1BB.

In some cases, the co-stimulatory domain is optional in the FAP-CAR.

In some cases, the FAP-CAR does not comprise a co-stimulatory domain, while the tumor-CAR does comprise a costimulatory domain such as the co-stimulatory domain from 4-1BB or from CD28.

As mentioned above, the FAP-CAR and tumor-CAR expressed by the engineered immune cells as described herewith have differential expressions as a result of having the exogenous nucleic acid sequence encoding a FAP-CAR placed under the transcriptional control of a constitutive promoter, and the exogenous nucleic acid sequence encoding a tumor-CAR placed under the transcriptional control of an inducible promoter.

By “constitutive promoter” is generally meant a promoter that is active in all circumstances in a particular cell or cell type comprising said promoter. A constitutive promoter carries out the transcription of its associated gene continuously in the cell. The level of transcription of the gene associated with a constitutive promoter can vary but the transcript and, thus, the gene's product (when there is one) remain detectable. Examples of constitutive promoters include the human elongation factor 1α (EF1A) promoter, the cluster of differentiation 52 (CD52) promoter, the Glyceraldehyde-3-Phosphate Dehydrogenase (GAPDH) promoter, the human cytomegalovirus (CMV) promoter, the human phosphoglycerate promoter (hPGK) promoter, the RPBSA promoter, the human Ubiquitin C (UBC) promoter, the simian virus 40 (SV40) early promoter, the mouse phosphoglycerate kinase 1 (PGK) promoter, and the chicken β-Actin promoter coupled with CMV early enhancer (CAGG), the T Cell Receptor Alpha Constant region (TRAC or TCRA) promoter, the T Cell Receptor Beta Constant 1 region (TRBC or TCRB) promoter, the T cell receptor gamma constant region 1 or 2 (TRGC1 or TCRG1, TRGC2 or TCRG2) promoter, the T Cell Receptor Delta Constant region (TRDC or TCRD) promoter, the Beta-2-Microglobulin (B2M) promoter, the cluster of differentiation 5 (CD5) promoter, the CS1 (also called CD319, CRACC and SLAMF7) promoter, the cluster of differentiation 45 (CD45) promoter, the cluster of differentiation 4 (CD4) promoter, the cluster of differentiation 8 (CD8) promoter. A constitutive promoter useful herewith can be identical to a promoter already present (i.e. without genetic engineering as described herewith) in the cell's genome. This would be the case for the EF1A promoter, the CD52 promoter, the GAPDH promoter, the TRAC promoter, the TRBC promoter, the TRGC promoter, the TRDC promoter, the B2M promoter, and the CD5 promoter, for example. The constitutive promoter useful herewith can also be absent from the cell's genome prior to its introduction in the cell by genetic engineering such as would be the case for the synthetic RPBSA promoter (that is a synthetic promoter made up of a fragment of the RPL13a promoter fused to a region of the RPL41 gene), the CMV promoter, the mouse PGK promoter, the SV40 promoter, the CAGG promoter. The constitutive promoter can be added to the cell's genome as an exogenous polynucleotide or can be an endogenous polynucleotide already present in the cell's genome independently of the cell's genetic engineering as described herewith, i.e. without addition to the cell of an exogenous polynucleotide corresponding to this promoter.

By “inducible promoter” is generally meant a promoter that becomes active in the cell comprising said promoter only in response to a specific stimulus. Therefore, an inducible promoter is active only under certain circumstances. Unless it receives a stimulus, the inducible promoter stays in an inactive state and the gene associated with the inducible promoter that is in the “off” state is generally not transcribed, or only weakly. Once the specific stimulus is present, the activator protein binds with the inducible promoter and makes it active to initiate transcription, it is in the “on” state. The transcription of the gene associated with the inducible promoter increases when the inducible promoter passes to an “on” state in response to the specific stimulus. The expression of a gene controlled by an inducible promoter is tightly regulated and its expression decreases rapidly upon removal of the activation signal. In the present disclosure, an inducible promoter is responsive to the cell activation (such as an immune cell, e.g. a T-cell, activation) as defined herewith. For example, promoters that are inducible upon CAR-T cell activation in vitro (e.g. as described in Example 6) fulfil the following criteria: fold change between average expression at 0 hours and average expression at 24 hours is greater than 3, for instance greater than 5.

By “activation of a cell” is generally meant the process by which changes occur in a cell in response to an “activation signal”. An “activation signal” designates a signal or stimulus that is able to, directly or indirectly, activate a cell. In the present disclosure, “activation of a cell”, when applied to an engineered cell comprising a CAR as described herewith, mainly refer to the changes occurring in said engineered cell after an activation signal is generated in the cell upon binding or recognition of an epitope of the FAP by the FAP-CAR expressed by said engineered cell and/or upon binding or recognition of an epitope of the tumor antigen by the tumor-CAR expressed by said engineered cell.

At the molecular level, the activation of a cell also corresponds to the activation of inducible promoters. Indeed, an inducible promoter comprises one or more regulatory elements responsive to one or more signaling pathways in the cell, such as the NFAT-regulated signal transduction.

In some cases, the inducible promoter can be responsive to the CD3 zeta signaling. Examples of inducible promoters useful herewith include the promoter of the Programmed Cell Death Protein 1 (PDCD1) gene, Cluster of Differentiation 25 (CD25) gene, T-cell immunoglobulin and mucin-domain containing-3 (TIM3) gene, T Cell Immunoreceptor With Ig And ITIM Domains (TIGIT) gene, C-C Motif Chemokine Ligand 1 (CCL1) gene, Nuclear Receptor Subfamily 4 Group A Member 3 (NR4A3) gene, Early Growth Response 3 (EGR3) gene, G0/G1 Switch 2 (GOS2) gene, Interleukin 22 (IL22) gene, Regulator of G Protein Signaling 16 (RGS16) gene, Fas Ligand (FASLG) gene, Retinol Dehydrogenase 10 (RDH10) gene, Colony Stimulating Factor 1 (CSF1) gene, Colony Stimulating Factor 2 (CSF2, also called GM-CSF) gene, Lymphocyte Activating 3 (LAG3) gene, Cytotoxic T-Lymphocyte Associated Protein 4 (CTLA-4 or CD152) gene, Interleukin-10 (IL10) gene, Nuclear Receptor Subfamily 4 Group A Member 1 (NR4A1 or NUR77) gene, Forkhead Box P3 (FOXP3) gene, and at least one NFAT responsive element. The inducible promoter useful herewith can be identical to a promoter already present (i.e. without genetic engineering as described herewith) in the cell's genome. This would be the case for the promoter of PDCD1 or the promoter of GM-CSF, for example. The inducible promoter useful herewith can also be absent from the cell's genome prior to its introduction in the cell by genetic engineering. The inducible promoter can be added to the cell's genome as an exogenous polynucleotide or can be an endogenous polynucleotide already present in the cell's genome independently of the cell's genetic engineering as described herewith, i.e. without addition to the cell of an exogenous polynucleotide corresponding to this promoter. As used herewith, the term “NFAT promoter” covers a polynucleotide sequence acting as a promoter and comprising at least one NFAT responsive element such as those described in Hooijberg et al. (Blood (2000) 96:459-466), Zhang et al. (Mol. Ther. (2011) 19, 751-759), or Takeuchi et al (J. Immunol. (1998) 160 (1): 209-218). A NFAT promoter or NFAT responsive elements can comprise, for instance, the nucleotide sequence GGAGGAAAAACTGTTTCATACAGAAGGCGT (SEQ ID NO: 114) or GGAGGAAAAACTGTTTCATACACAGAAGGCCT (SEQ ID NO: 115). The NFAT responsive element can be repeated, for instance 3, 6, 9 or more times.

In some cases, following an activation signal in the engineered cells as described herewith, the frequency of cells expressing the tumor-CAR in the cell population comprising said engineered immune cells is at least about 10%, 15%, 20%, 30%, 40%, 50%, 60%, 70%, 75%, 80%, 85%, 90% or 95%.

In some cases, the expression of the tumor-CAR that was induced by the activation signal in the engineered cells returns to its initial basal level or is not detectable after removal, disappearance, or reduction of the activation signal in said engineered immune cells.

### 1.3. Further Features of the Engineered Cells

In some cases, the engineered immune cells, e.g. T-cells, that have been modified to express a CAR directed against FAP and a CAR directed against the tumor antigen can have one or more additional modifications.

Additional genetic attributes may be conferred by gene editing the immune cells in order to improve their therapeutic potency.

In some cases, the engineered cell can be further modified to improve its persistence or its lifespan into the patient, for instance inactivating a gene encoding MHC-I component(s) such as HLA or B2M, such as described in WO 2015/136001 or by Liu et al. (2017, Cell Res 27:154-157).

Beta-2 microglobulin, also known as B2m, is the light chain of MHC class I molecules, and as such an integral part of the major histocompatibility complex. In human, β2m is encoded by the B2M gene which is located on chromosome 15, as opposed to the other MHC genes which are located as gene cluster on chromosome 6. The human protein is composed of 119 amino acids and has a molecular weight of 11,800 Daltons.

In some cases, inhibition of expression of B2M is achieved by a genome modification, such as through the expression in the cell of a rare-cutting endonuclease able to selectively inactivate by DNA cleavage the gene encoding β2m, such as the human B2M gene (NCBI Reference Sequence: NG_012920.1). Such rare-cutting endonuclease may be a TALE-nuclease, meganuclease, zing-finger nuclease (ZFN), or RNA guided endonuclease (such as Cas9).

In some cases, inhibition of expression of B2M can be achieved by using (e.g. introducing into the T-cell) a nucleic acid molecule that specifically hybridizes (e.g. binds) under cellular conditions with the cellular mRNA and/or genomic DNA encoding β2m, thereby inhibiting transcription and/or translation of the gene. In some cases, the inhibition of expression of B2M is achieved by using (e.g. introducing into the T-cell) an antisense oligonucleotide, ribozyme or interfering RNA (RNAi) molecule. In some cases, such nucleic acid molecule can comprise at least 10 consecutive nucleotides of the complement of the mRNA encoding human β2m.

In some cases, an immune cell (e.g. a T-cell) or a precursor cell is provided which expresses a rare-cutting endonuclease able to selectively inactivate by DNA cleavage the gene encoding β2m. For instance, such cell comprises an exogenous nucleic acid molecule comprising a nucleotide sequence encoding said rare-cutting endonuclease, which may be a TALE-nuclease, meganuclease, zing-finger nuclease (ZFN), or RNA guided endonuclease. Thus, in order to provide less alloreactive immune cells (e.g. T-cells), the method described herewith can further comprise the step of inactivating or mutating B2M gene.

In some cases, the engineered immune cells, e.g. T-cells, have been modified to suppress or repress expression of HLA in said cells. The class I HLA gene cluster in humans comprises three major loci, B, C and A, as well as several minor loci. The class II HLA cluster also comprises three major loci, DP, DQ and DR, and both the class I and class II gene clusters are polymorphic, in that there are several different alleles of both the class I and II genes within the population. There are also several accessory proteins that play a role in HLA functioning as well. The Tap1 and Tap2 subunits are parts of the TAP transporter complex that is essential in loading peptide antigens on to the class I HLA complexes, and the LMP2 and LMP7 proteosome subunits play roles in the proteolytic degradation of antigens into peptides for display on the HLA. Reduction in LMP7 has been shown to reduce the amount of MHC class I at the cell surface, perhaps through a lack of stabilization (Fehling et al. (1999) Science 265:1234-1237). In addition to TAP and LMP, there is the tapasin gene, whose product forms a bridge between the TAP complex and the HLA class I chains and enhances peptide loading. Reduction in tapasin results in cells with impaired MHC class I assembly, reduced cell surface expression of the MHC class I and impaired immune responses (Grandea et al. (2000) Immunity 13:213-222 and Garbi et al. (2000) Nat. Immunol. 1:234-238). Any of the above genes may be inactivated as part of the present document as disclosed, for instance in WO 2012/012667.

In some cases, the engineered immune cells, e.g. T-cells, have been modified to suppress or repress expression of CIITA in said cells. CIITA is the gene encoding class II major histocompatibility complex transactivator protein.

In some cases, the engineered immune cells, e.g. T-cells, are inactivated in at least one gene selected from the group consisting of RFXANK, RFX5, RFXAP, TAP1, TAP2, ZXDA, ZXDB and ZXDC. Inactivation may, for instance, be achieved by using a genome modification, such as through the expression in the cell of a rare-cutting endonuclease able to selectively inactivate, by DNA cleavage, a gene selected from the group consisting of RFXANK, RFX5, RFXAP, TAP1, TAP2, ZXDA, ZXDB and ZXDC. Such modifications can permit the engineered immune cells to be less alloreactive when infused into patients.

Thus, in one aspect, the engineered cells described herewith can be genetically modified to suppress or repress expression of at least one gene controlling MHC complex surface presentation. A gene controlling MHC complex surface presentation as defined herewith includes B2M, CIITA, HLA, RFXANK, RFX5, RFXAP, TAP1, TAP2, ZXDA, ZXDB and ZXDC. In some cases, said engineered immune cells, e.g. T-cells or NK-cells, have been genetically modified to suppress or repress expression of a gene encoding an immune checkpoint protein and/or the receptor thereof, in said cells, such as PDCD1 or CTLA4 as described in WO 2014/184744.

It will be understood by those of ordinary skill in the art, that the term “immune checkpoints” means a group of molecules expressed by T-cells, NK-cells and antigen presenting cells. These molecules effectively serve as “brakes” to down-modulate or inhibit an immune response. Immune checkpoint molecules include, but are not limited to Programmed Death 1 (PD-1, also known as PDCD1 or CD279, e.g. human PD-1: accession number NM_005018), Cytotoxic T-Lymphocyte Antigen 4 (CTLA-4, also known as CD152, e.g. human CTLA-4: GenBank accession number AF414120.1), LAG3 (also known as CD223, e.g. human LAG3: accession number NM_002286.5), Tim3 (also known as HAVCR2, e.g. human Tim3: GenBank accession number JX049979.1), BTLA (also known as CD272, e.g. human BTLA: accession number NM_181780.3), BY55 (also known as CD160, e.g. human BY55: GenBank accession number CR541888.1), TIGIT (also known as IVSTM3, e.g. human TIGIT: accession number NM_173799), LAIR1 (also known as CD305, e.g. human LAIR1: GenBank accession number CR542051.1), SIGLEC10 (e.g. human SIGLEC10: GeneBank accession number AY358337.1), 2B4 (also known as CD244, e.g. human 2B4: accession number NM_001166664.1), PPP2CA (Also known as: NEDLBA, PP2Ac, PP2Calpha, RP-C, e.g. human PPP2CA: NCBI Gene ID 5515), PPP2CB (also known as Also known as: PP2Abeta, e.g. human PPP2CB: NCBI Gene ID 5516), PTPN6 (also known as Also known as: HCP, HCPH, HPTPIC, PTP-1C, SH-PTP1, SHP-1, SHP-1L, SHP1, e.g. human PTPN6: NCBI Gene ID 5777), PTPN22 (NCBI Gene ID 26191), CD96 (NCBI Gene ID 10225), CRTAM (NCBI Gene ID 56253), SIGLEC7 (NCBI Gene ID 27036), SIGLEC9 (NCBI Gene ID 27180), TNFRSF10B (NCBI Gene ID 8795), TNFRSF10A (NCBI Gene ID 8797), CASP8 (NCBI Gene ID 841), CASP10 (NCBI Gene ID 843), CASP3 (NCBI Gene ID 836), CASP6 (NCBI Gene ID 839), CASP7 (NCBI Gene ID 840), FADD (NCBI Gene ID 8772), FAS (NCBI Gene ID 355), TGFBRII (NCBI Gene ID 7048), TGFRBRI (NCBI Gene ID 7046), SMAD2 (NCBI Gene ID 4087), SMAD3 (NCBI Gene ID 4088), SMAD4 (NCBI Gene ID 4089), SMAD10, SKI (NCBI Gene ID 6497), SKIL (NCBI Gene ID 6498), TGIF1 (NCBI Gene ID 7050), IL10RA (NCBI Gene ID 3587), IL10RB (NCBI Gene ID 3588), HMOX2 (NCBI Gene ID 3163), IL6R (NCBI Gene ID 3570), IL6ST (NCBI Gene ID 3572), EIF2AK4 (NCBI Gene ID 440275), CSK (NCBI Gene ID 1445), PAG1 (NCBI Gene ID 55824), SIT1 (NCBI Gene ID 27240), FOXP3 (NCBI Gene ID 50943), PRDM1 (NCBI Gene ID 639), BATF (NCBI Gene ID 10538), GUCY1A2 (NCBI Gene ID 2977), GUCY1A3 (NCBI Gene ID 2977), and GUCY1B2 (NCBI Gene ID 2974) which directly inhibit immune cells. For example, CTLA-4 is a cell-surface protein expressed on certain CD4 and CD8 T-cells; when engaged by its ligands (B7-1 and B7-2) on antigen presenting cells, T-cell activation and effector function are inhibited. In some cases, the engineered T-cells are further genetically modified by inactivating at least one gene encoding a protein involved in the immune checkpoint, such as PD1 and/or CTLA-4 or any immune-checkpoint proteins referred to herein.

In some cases, at least two genes encoding immune checkpoint proteins are inactivated, selected from the group consisting of: CTLA4, PPP2CA, PPP2CB, PTPN6, PTPN22, PDCD1, LAG3, HAVCR2, BTLA, CD160, TIGIT, CD96, CRTAM, LAIR1, SIGLEC7, SIGLEC9, CD244, TNFRSF10B, TNFRSF10A, CASP8, CASP10, CASP3, CASP6, CASP7, FADD, FAS, TGFBRII, TGFRBRI, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, GUCY1A2, GUCY1A3, GUCY1B2, and GUCY1B3.

In some cases, the engineered immune cells, e.g. T-cells, can be modified to confer resistance to at least one immune suppressive or chemotherapy drug, and optionally to comprise a suicide gene.

In some cases, the engineered immune cells, e.g. T-cells, can be further modified to confer resistance to at least one immune suppressive drug, such as by inactivating CD52 that is the target of anti-CD52 antibody (e.g. alemtuzumab), as described for instance in WO 2013/176915.

To improve cancer therapy and selective engraftment of allogeneic immune cells, drug resistance can be conferred to the engineered immune cells to protect them from the toxic side effects of chemotherapy or immunosuppressive agents. In some cases, the engineered immune cell can be further modified to confer resistance to a chemotherapy drug, such as a purine analogue drug, for example by inactivating DCK as described in WO 2015/75195.

Drug resistance of immune cells also permits their enrichment in or ex vivo, as immune cells which express a drug resistance gene, will survive and multiply relative to drug sensitive cells. In some cases, the methods further comprise methods of engineering allogeneic and drug-resistant immune cells for immunotherapy comprising: (a) providing an immune cell, e.g. a T-cell; (b) selecting at least one drug; (c) modifying the cell to confer drug resistance to said cell; and (d) expanding said engineered cell in the presence of said drug. When the immune cell is a T-cell, the preceding steps may be combined with a step of modifying the T-cell, by inactivating at least one gene encoding a T-cell receptor (TCR) component, and then sorting the transformed T-cells, which do not express TCR on their cell surface.

Thus, the engineered immune cells can be further modified to confer a resistance to a drug, such as a chemotherapy agent. The resistance to a drug can be conferred to an immune cell by expressing a drug resistance gene. Variant alleles of several genes such as dihydrofolate reductase (DHFR), inosine monophosphate dehydrogenase 2 (IMPDH2), calcineurin or methylguanine transferase (MGMT) have been identified to confer drug resistance to a cell. In some cases, the drug resistance gene can be expressed in the cell either by introducing a transgene encoding said gene into the cell or by integrating said drug resistance gene into the genome of the cell by homologous recombination.

The resistance to a drug can be conferred to an immune cell by inactivating one or more gene(s) responsible for the cell's sensitivity to the drug (drug sensitizing gene(s)), such as the hypoxanthine-guanine phosphoribosyl transferase (HPRT) gene (Genbank: M26434.1). For instance, HPRT can be inactivated in engineered immune cells to confer resistance to a cytostatic metabolite, the 6-thioguanine (6TG) which is converted by HPRT to cytotoxic thioguanine nucleotide and which is currently used to treat patients with cancer, in particular leukemias (Hacke et al. (2013) Transplantation Proceedings, 45 (5): 2040-2044). Another example is the inactivation of the CD3 normally expressed at the surface of the T-cell, which can confer resistance to anti-CD3 antibodies such as teplizumab.

Otherwise, drug resistance can be conferred to the immune cell (e.g. T-cell), by the expression of at least one drug resistance gene. The drug resistance gene refers to a nucleic acid sequence that encodes “resistance” to an agent, such as a chemotherapeutic agent (e.g. methotrexate). In other words, the expression of the drug resistance gene in a cell permits proliferation of the cells in the presence of the agent to a greater extent than the proliferation of a corresponding cell without the drug resistance gene. A drug resistance gene can encode resistance to anti-metabolite, methotrexate, vinblastine, cisplatin, alkylating agents, anthracyclines, cytotoxic antibiotics, anti-immunophilins, their analogs or derivatives, and the like.

Several drug resistance genes have been identified that can potentially be used to confer drug resistance to targeted cells (Takebe et al. (2001) Mol. Ther. 3 (1): 88-96); Sugimoto et al. (2003) Mol Cancer Ther. 2:105-112; Zielske et al. (2003) J. Clin. Invest. 112 (10): 1561-70; Nivens et al. (2004) Cancer Chemother Pharmacol 53 (2): 107-15; Bardenheuer et al. (2005) Leukemia 19 (12): 2281-8; Kushman et al. (2007) Carcinogenesis 28 (1): 207-14).

One example of drug resistance gene can also be a mutant or modified form of Dihydrofolate reductase (DHFR). DHFR is an enzyme involved in regulating the amount of tetrahydrofolate in the cell and is essential to DNA synthesis. Folate analogs such as methotrexate (MTX) inhibit DHFR and are thus used as anti-neoplastic agents in clinic. Different mutant forms of DHFR which have increased resistance to inhibition by anti-folates used in therapy have been described. In some cases, the drug resistance gene can be a nucleic acid sequence encoding a mutant form of human wild type DHFR (GenBank: AAH71996.1) which comprises at least one mutation conferring resistance to an anti-folate treatment, such as methotrexate. In some cases, mutant form of DHFR comprises at least one mutated amino acid at position G15, L22, F31 or F34, for instance at positions L22 or F31 (Schweitzer, Dicker et al. 1990); International application WO94/24277; U.S. Pat. No. 6,642,043).

As used herein, “antifolate agent” or “folate analogs” refers to a molecule directed to interfere with the folate metabolic pathway at some level. Examples of antifolate agents include, e.g. methotrexate (MTX); aminopterin; trimetrexate (Neutrexin™); edatrexate; N10-propargyl-5,8-dideazafolic acid (CB3717); ZD1694 (Tumodex), 5,8-dideazaisofolic acid (IAHQ); 5,10-dideazatetrahydrofolic acid (DDATHF); 5-deazafolic acid; PT523 (N alpha-(4-amino-4-deoxypteroyl)-N delta-hemiphthaloyl-L-ornithine); 10-ethyl-10-deazaaminopterin (DDATHF, Iomatrexol); piritrexim; 10-EDAM; ZD1694; GW1843; Pemetrexate and PDX (10-propargyl-10-deazaaminopterin).

Another example of drug resistance gene can also be a mutant or modified form of ionisine-5′-monophosphate dehydrogenase II (IMPDH2), a rate-limiting enzyme in the de novo synthesis of guanosine nucleotides. The mutant or modified form of IMPDH2 is a IMPDH inhibitor resistance gene. IMPDH inhibitors can be mycophenolic acid (MPA) or its prodrug mycophenolate mofetil (MMF). The mutant IMPDH2 can comprise at least one, for instance two mutations in the MAP binding site of the wild type human IMPDH2 (NP_000875.2) that lead to a significantly increased resistance to IMPDH inhibitor. The mutations can be at positions T333 and/or S351 (Yam et al. (2006) Mol. Ther. 14 (2): 236-44; Jonnalagadda et al. (2013) PLoS One 8 (6): e65519). In some cases, the threonine residue at position 333 can be replaced with an isoleucine residue and the serine residue at position 351 can be replaced with a tyrosine residue.

Another drug resistance gene is the mutant form of calcineurin. Calcineurin (PP2B) is an ubiquitously expressed serine/threonine protein phosphatase that is involved in many biological processes and which is central to T-cell activation. Calcineurin is a heterodimer composed of a catalytic subunit (CnA; three isoforms) and a regulatory subunit (CnB; two isoforms). After engagement of the T-cell receptor, calcineurin dephosphorylates the transcription factor NFAT, allowing it to translocate to the nucleus and active key target gene such as 1L2. FK506 in complex with FKBP12, or cyclosporine A (CsA) in complex with CyPA block NFAT access to calcineurin's active site, preventing its dephosphorylation and thereby inhibiting T-cell activation (Brewin et al. (2009) Blood 114 (23): 4792-803). The drug resistance gene can be a nucleic acid sequence encoding a mutant form of calcineurin resistant to calcineurin inhibitor such as FK506 and/or CsA. In some cases, said mutant form can comprise at least one mutated amino acid of the wild type calcineurin heterodimer at positions: V314, Y341, M347, T351, W352, L354, K360, for instance double mutations at positions T351 and L354 or V314 and Y341. Correspondence of amino acid positions described herein is frequently expressed in terms of the positions of the amino acids of the form of wild-type human calcineurin heterodimer (GenBank: ACX34092.1).

In some cases, said mutant form can comprise at least one mutated amino acid of the wild type calcineurin heterodimer b at positions: V120, N123, L124 or K125, for instance double mutations at positions L124 and K125. Correspondence of amino acid positions described herein is frequently expressed in terms of the positions of the amino acids of the form of wild-type human calcineurin heterodimer b polypeptide (GenBank: ACX34095.1).

Another drug resistance gene is O⁶-methylguanine methyltransferase (MGMT) encoding human alkyl guanine transferase (hAGT). AGT is a DNA repair protein that confers resistance to the cytotoxic effects of alkylating agents, such as nitrosoureas and temozolomide (TMZ). 6-benzylguanine (6-BG) is an inhibitor of AGT that potentiates nitrosourea toxicity and is co-administered with TMZ to potentiate the cytotoxic effects of this agent. Several mutant forms of MGMT that encode variants of AGT are highly resistant to inactivation by 6-BG, but retain their ability to repair DNA damage (Maze, Kurpad et al. 1999). In some cases, AGT mutant form can comprise a mutated amino acid of the wild type AGT position P140 (UniProtKB: P16455).

Another drug resistance gene can be multidrug resistance protein 1 (MDR1) gene. This gene encodes a membrane glycoprotein, known as P-glycoprotein (P-GP) involved in the transport of metabolic byproducts across the cell membrane. The P-Gp protein displays broad specificity towards several structurally unrelated chemotherapy agents. Thus, drug resistance can be conferred to cells by the expression of nucleic acid sequence that encodes MDR-1 (NP_000918).

Drug resistance genes can also be cytotoxic antibiotics, such as ble gene or mcrA gene. Ectopic expression of ble gene or mcrA in an immune cell gives a selective advantage when exposed to the chemotherapeutic agent, respectively the bleomycine or the mitomycin C.

With respect to the immunosuppressive agents, the present document describes the possible optional steps of: (a) providing an immune cell such as a T-cell, for instance from a cell culture or from a blood sample, or an induced pluripotent stem cell (iPSC); (b) selecting a gene in said cell expressing a target for an immunosuppressive agent; (c) introducing into said cell an endonuclease able to selectively inactivate by DNA cleavage, for instance by double-strand break, said gene encoding a target for said immunosuppressive agent, (d) expanding said cells, optionally in presence of said immunosuppressive agent. In some cases, said method comprises a further step of inactivating a component of the T-cell receptor (TCR).

An immunosuppressive agent is an agent that suppresses immune function by one of several mechanisms of action. In other words, an immunosuppressive agent is a compound which is capable of diminishing the extent and/or voracity of an immune response. As non-limiting examples, an immunosuppressive agent can be a calcineurin inhibitor, a target of rapamycin, an interleukin-2α-chain blocker, an inhibitor of inosine monophosphate dehydrogenase, an inhibitor of dihydrofolic acid reductase, a corticosteroid or an immunosuppressive antimetabolite. Classical cytotoxic immunosuppressants act by inhibiting DNA synthesis. Others may act through inactivation of T-cells or by inhibiting the activation of helper cells. The method described herewith allows conferring immunosuppressive resistance to immune cells (e.g. T-cells), for immunotherapy by inactivating the target of the immunosuppressive agent in said cells. As non-limiting examples, a target for an immunosuppressive agent can be a receptor for an immunosuppressive agent such as CD52, glucocorticoid receptor (GR), a FKBP family gene member and a cyclophilin family gene member.

In immunocompetent hosts, allogeneic cells are normally rapidly rejected by the host immune system. It has been demonstrated that allogeneic leukocytes present in non-irradiated blood products will persist for no more than 5 to 6 days (Boni et al. (2008) Blood 112 (12): 4746-54). Thus, to prevent rejection of allogeneic cells, the host's immune system must be effectively suppressed. Glucocorticoid steroids are widely used therapeutically for immunosuppression (Coutinho and Chapman (2011) Mol. Cell Endocrinol. 335 (1): 2-13). This class of steroid hormones binds to the glucocorticoid receptor (GR) present in the cytosol of T-cells resulting in the translocation into the nucleus and the binding of specific DNA motifs that regulate the expression of a number of genes involved in the immunologic process. Treatment of T-cells with glucocorticoid steroids results in reduced levels of cytokine production leading to T-cell anergy and interfering in T-cell activation. Alemtuzumab, also known as CAMPATH1-H, is a humanized monoclonal antibody targeting CD52, a 12 amino acid glycosylphosphatidyl-inositol-(GPI) linked glycoprotein (Waldmann and Hale (2005) Philos. Trans. R. Soc. Lond. B. Biol Sci. 360:1701-11). CD52 is expressed at high levels on T and B lymphocytes and lower levels on monocytes while being absent on granulocytes and bone marrow precursors. Treatment with Alemtuzumab, a humanized monoclonal antibody directed against CD52, has been shown to induce a rapid depletion of circulating lymphocytes and monocytes. It is frequently used in the treatment of T-cell lymphomas and in certain cases as part of a conditioning regimen for transplantation. However, in the case of adoptive immunotherapy the use of immunosuppressive drugs will also have a detrimental effect on the introduced therapeutic immune cells (e.g. T-cells). Therefore, to effectively use an adoptive immunotherapy approach in these conditions, the introduced cells would need to be resistant to the immunosuppressive treatment.

In some cases, the gene that is specific for an immunosuppressive treatment is CD52, and the immunosuppressive treatment comprises a humanized antibody targeting CD52 antigen. In some cases, the gene that is specific for an immunosuppressive treatment is a glucocorticoid receptor (GR) and the immunosuppressive treatment comprises a corticosteroid such as dexamethasone. In some cases, the gene that is specific for an immunosuppressive treatment is a FKBP family gene member or a variant thereof and the immunosuppressive treatment comprises FK506 also known as Tacrolimus or fujimycin. In some cases, the gene that is specific for an immunosuppressive treatment is a FKBP family gene member such as FKBP12 or a variant thereof. In some cases, the gene that is specific for an immunosuppressive treatment is a cyclophilin family gene member or a variant thereof and the immunosuppressive treatment comprises cyclosporine.

Cytokine Release Syndrome (CRS) is the most common adverse event of CAR-T cell therapy. CRS is defined as a clinical syndrome that may occur after cell therapy due to the release of cytokines (substances secreted by immune cells) into the body's blood stream. It has been shown that inactivation of Granulocyte-macrophage colony-stimulating factor (GM-CSF) can prevent monocyte-dependent release of key cytokine release syndrome mediators (Sachdeva et al. (2019) J. Biol. Chem. 294 (14) 5430-5437). Thus, in a further aspect, the engineered immune cells as described herewith have been genetically modified to suppress expression, or cell surface presentation, of GM-CSF.

In some cases, the engineered immune cell as described herewith is one or more of: TCR negative, B2M negative, CIITA negative, PDCD1 negative, GM-CSF negative, CD52 negative; for instance at least TCR negative or at least TCR negative, B2M-negative and CD52-negative.

In some cases, to reduce fratricide effect, the engineered immune cell as described herewith does not present at its cell surface the antigen targeted by the tumor-CAR. For example, the engineered immune cell as described herewith can have its CD4 or CD8 gene inactivated, or its expression inhibited, if the tumor-CAR targets CD4 or CD8, respectively.

### 2. Methods of Producing the Engineered Cells as Described Herewith

Another aspect provides a method of producing a population of cells comprising engineered immune cells as described herewith, comprising:
(i) providing immune cells from a donor or providing induced pluripotent stem cells (iPSCs);
(ii) optionally, inactivating the potential expression of a T-Cell Receptor (TCR) in the cells or its presentation at the cells' surface;
(iii) integrating in the cells' genome an exogenous nucleic acid sequence encoding a chimeric antigen receptor (CAR) targeting the Fibroblast Activation Protein. (FAP) (“FAP-CAR”) placed under the transcriptional control of an exogenous or endogenous constitutive promoter; and
(iv) integrating in the cells' genome an exogenous nucleic acid sequence encoding a chimeric antigen receptor (CAR) targeting a tumor antigen (“tumor-CAR”) placed under the transcriptional control of an exogenous or endogenous inducible promoter;
(v) optionally, differentiating the engineered iPSCs into immune cells;
(vi) optionally, isolating the engineered cells which do not express a TCR at their cell surface;
wherein the expression of the tumor-CAR is inducible upon activation of the immune cells.

The source of the cells provided in step (i), i.e. to be engineered, is not particularly limiting. In some cases, the cells of step (i) can be immune cells originating from a donor or resulting from the differentiation of iPSCs into immune cells. The cells of step (i) can also be iPSCs, which can be differentiated into immune cells after any one of the steps of genetic engineering (ii) to (iv) as disclosed above.

By “immune cell” is meant a cell of hematopoietic origin functionally involved in the initiation and/or execution of innate and/or adaptative immune response, such as typically CD45, CD3, CD8 or CD4 positive cells. Immune cells include dendritic cells, killer dendritic cells, mast cells, macrophages, natural killer cells (NK-cell), cytokine-induced killer cells (CIK cells), B-cells or T-cells selected from the group consisting of inflammatory T-lymphocytes, cytotoxic T-lymphocytes, or helper T-lymphocytes, gamma delta T-cells, and Natural killer T-cells (“NKT cell).

In some cases, the source of the immune cells (such as T-cells) to be engineered are primary cells, and by “primary cell(s)” are intended cells taken directly from living tissue (e.g. biopsy material) and established for growth in vitro for a limited amount of time, meaning that they can undergo a limited number of population doublings. Primary cells are opposed to continuous tumorigenic or artificially immortalized cell lines. Non-limiting examples of such cell lines are CHO-K1 cells; HEK293 cells; Caco2 cells; U2-OS cells; NIH 3T3 cells; NSO cells; SP2 cells; CHO-S cells; DG44 cells; K-562 cells, U-937 cells; MRC5 cells; IMR90 cells; Jurkat cells; HepG2 cells; HeLa cells; HT-1080 cells; HCT-116 cells; Hu-h7 cells; Huvec cells; and Molt 4 cells.

Primary immune cells can be obtained from a number of non-limiting sources, including peripheral blood mononuclear cells (PBMC), bone marrow, lymph node tissue, cord blood, thymus tissue, tissue from a site of infection, ascites, pleural effusion, spleen tissue, and from tumors, such as tumor infiltrating lymphocytes. In some cases, said immune cell can be derived from a healthy donor, from a patient diagnosed with cancer or from a patient diagnosed with an infection. In some cases, said cell is part of a mixed population of immune cells which present different phenotypic characteristics, such as comprising CD4, CD8 and CD56 positive cells. Primary immune cells are provided from donors or patients through a variety of methods known in the art, as for instance by leukapheresis techniques as reviewed by Schwartz J. et al. (Guidelines on the use of therapeutic apheresis in clinical practice-evidence-based approach from the Writing Committee of the American Society for Apheresis: the sixth special issue (2013) J Clin Apher. 28 (3): 145-284).

In the present document, are also regarded as primary immune cells the immune cells derived from stem cells, such as those deriving from induced pluripotent stem cells (iPSCs) (Yamanaka, K. et al. (2008) Science. 322 (5903): 949-53). Lentiviral expression of reprogramming factors has been used to induce multipotent cells from human peripheral blood cells (Staerk et al. (2010) Cell stem cell. 7 (1): 20-4; Loh et al. (2010) Cell stem cell. 7 (1): 15-9).

According to some cases, the immune cells can be derived from human embryonic stem cells by techniques well known in the art that do not involve the destruction of human embryos (Chung et al. (2008) Cell Stem Cell 2 (2): 113-117).

In some cases, the engineered T-cells can derive from inflammatory T-lymphocytes, cytotoxic T-lymphocytes, or helper T-lymphocytes.

In some cases, the immune cell, e.g. T-cell or NK-cell, can derive from a stem cell. The stem cells can be adult stem cells, embryonic stem cells, such as non-human stem cells, cord blood stem cells, progenitor cells, bone marrow stem cells, induced pluripotent stem cells, totipotent stem cells or hematopoietic stem cells. Representative human cells are CD34+ cells.

In some cases, the engineered cells can derive from the group consisting of CD4+T-lymphocytes and CD8+T-lymphocytes. Prior to their expansion and genetic modification, the cells can be obtained from a subject through a variety of non-limiting methods. T-cells can be obtained from a number of non-limiting sources, including peripheral blood mononuclear cells, bone marrow, lymph node tissue, cord blood, thymus tissue, tissue from a site of infection, ascites, pleural effusion, spleen tissue, and tumors. In certain cases, any number of T-cell lines available and known to those skilled in the art, may be used. In some cases, said cell can be derived from a healthy donor or from a patient diagnosed with cancer. In some cases, said cell is part of a mixed population of cells which present different phenotypic characteristics. In the scope of the disclosure is also encompassed a cell line obtained from a transformed T-cell according to the method previously described. Modified cells resistant to an immunosuppressive treatment and susceptible to be obtained by the previous method are also disclosed herewith.

In some cases, the immune cells (e.g. T-cells or NK cells) to be engineered are allogenic. By “allogeneic” is meant that the cells originate from a donor, from a cell line, or are produced and/or differentiated from stem cells in view of being infused into patients having a different haplotype. Such immune cells are generally engineered to be less alloreactive and/or become more persistent with respect to their patient host. More specifically, the method of engineering the allogeneic cells can comprise the step of reducing or suppressing TCR expression into T-cells, or into the stem cells to be derived into T-cells. This can be obtained by different sequence-specific reagents, such as by gene silencing or gene editing techniques by using for instance nucleases, base editing techniques, shRNA and RNAi as non-limited examples.

In some cases, the immune cells, e.g. T-cells or NK-cells, to be engineered can originate from a human, wherein the human is a donor, not the patient.

In some cases, the engineered T-cells can comprise an inactivated T-cell receptor (TCR) and can have been modified by inactivating at least one component of the TCR, e.g. by using a sequence-specific endonuclease such as a RNA guided endonuclease associated with a specific guide RNA, or using other gene editing approaches such as TALE-nucleases. T cell receptors (TCR) are cell surface receptors that participate in the activation of T-cells in response to the presentation of antigen. The TCR is generally made from two chains, alpha and beta, which assemble to form a heterodimer and associates with the CD3-transducing subunits to form the T-cell receptor complex present on the cell surface. Each alpha and beta chain of the TCR consists of an immunoglobulin-like N-terminal variable (V) and constant (C) region, a hydrophobic transmembrane domain, and a short cytoplasmic region. As for immunoglobulin molecules, the variable region of the alpha and beta chains are generated by V (D) J recombination, creating a large diversity of antigen specificities within the population of T-cells. However, in contrast to immunoglobulins that recognize intact antigen, T-cells are activated by processed peptide fragments in association with an MHC molecule, introducing an extra dimension to antigen recognition by T-cells, known as MHC restriction. Recognition of MHC disparities between the donor and recipient through the T cell receptor leads to T-cell proliferation and the potential development of GvHD. It has been shown that normal surface expression of the TCR depends on the coordinated synthesis and assembly of all seven components of the complex (Ashwell and Klusner (1990) Annu. Rev. Immunol. 8:139-67). The inactivation of TRAC (encoding TCRalpha constant domain) or TRBC (encoding TCRbeta constant domain) can result in the elimination of the TCR from the surface of T-cells preventing recognition of alloantigen and thus GVHD. However, TCR disruption generally results in the elimination of the CD3 signaling component and alters the means of further T-cell expansion.

In some cases, at least 50%, at least 70%, at least 90%, or at least 95% of said engineered T-cells in the population are mutated in their TRAC, TRBC and/or CD3 alleles.

In some cases, the TCR is inactivated by using specific TALE-nucleases, better known under the trademark TALEN® (Cellectis, 8, rue de 1a Croix Jarry, 75013 PARIS). This method has proven to be highly efficient in primary cells using RNA transfection as part of a platform allowing the mass production of allogeneic T-cells. See, e.g. WO 2013/176915, which is incorporated by reference herein in its entirety.

In some cases, the TCR is inactivated using an RNA guided endonuclease associated with a specific guide RNA. U.S. Pat. No. 10,870,864 describes methods for inactivating a TCR in cells using such methods, which is incorporated by reference herein. Engraftment of allogeneic T-cells is possible by inactivating at least one gene encoding a TCR component. In some cases, the TCR is rendered not functional in the cells by inactivating a TRAC gene and/or a TCRB gene. TCR inactivation in allogeneic T-cells aims to prevent or reduce GvHD.

In some cases, the TCR gene is inactivated by inserting into the TRAC locus of the cell's genome at least one exogenous polynucleotide encoding a FAP-CAR comprising: (a) an extracellular FAP-binding-domain comprising a Heavy Variable chain (VH) and a Light Variable chain (VL) from a monoclonal anti-FAP antibody, (b) a hinge selected from a FcγRIII hinge, a CD8α hinge and an IgG1 hinge, (c) a CD8α transmembrane domain or a CD28 transmembrane domain, and (d) a cytoplasmic domain including a CD3 zeta signaling domain and optionally a co-stimulatory domain from 4-1BB or from CD28.

By inactivating a gene, it is intended that the gene of interest is not expressed in a functional protein form. In some cases, genetic modification of the cells relies on the expression, in provided cells to engineer, of an endonuclease so that it catalyzes cleavage in one targeted gene thereby inactivating the targeted gene. The nucleic acid strand breaks caused by the endonuclease are commonly repaired through the distinct mechanisms of homologous recombination or non-homologous end joining (NHEJ). However, NHEJ is an imperfect repair process that often results in changes to the DNA sequence at the site of the cleavage. Mechanisms involve rejoining of what remains of the two DNA ends through direct re-ligation (Critchlow and Jackson (1998) Trends Biochem Sci. 23 (10): 394-8) or via the so-called microhomology-mediated end joining (Betts et al. (2003) J. Immunol. Methods 281 (1-2): 65-78; Ma et al. (2003) Mol Cell Biol 23 (23): 8820-8). Repair via non-homologous end joining (NHEJ) often results in small insertions or deletions and can be used for the creation of specific gene knockouts. The modification may be a substitution, deletion, or addition of at least one nucleotide. Cells in which a cleavage-induced mutagenesis event, i.e. a mutagenesis event consecutive to an NHEJ event, has occurred can be identified and/or selected by well-known methods in the art.

As the engineered immune cells described herewith can derive from the differentiation of engineered iPSCs as described herewith into said immune cells, another aspect described in the present application concerns a method of producing a population of cells comprising engineered iPSCs as described herewith, comprising:
(i) providing induced pluripotent stem cells (iPSCs);
(ii) optionally, inactivating the potential expression of a T-Cell Receptor (TCR) in the cells or its presentation at the cells' surface;
(iii) integrating in the cells' genome an exogenous nucleic acid sequence encoding a chimeric antigen receptor (CAR) targeting the Fibroblast Activation Protein (FAP) (“FAP-CAR”) placed under the transcriptional control of an exogenous or endogenous constitutive promoter; and
(iv) integrating in the cells' genome an exogenous nucleic acid sequence encoding a chimeric antigen receptor (CAR) targeting a tumor antigen (“tumor-CAR”) placed under the transcriptional control of an exogenous or endogenous inducible promoter;
wherein the expression of the tumor-CAR is inducible upon activation of the immune cell into which said engineered iPSC can further be differentiated.

Another aspect, thus, includes a method of producing a population of cells comprising engineered immune cells as described herewith, comprising (i) producing a population of cells comprising engineered iPSCs as described above, and (ii) differentiating said engineered iPSCs into immune cells.

### Engineering and Gene Editing

The methods that can be employed herein to engineer or gene edit cells are not particularly limiting. In some cases, the cells can be contacted with a sequence-specific reagent to modify (e.g. engineer or gene edit) the cells.

By “sequence-specific reagent” is meant any active molecule that has the ability to specifically recognize a selected polynucleotide sequence at a genomic locus, referred to as “target sequence,” which is generally of at least 12 bp, at least 15 bp, or at least 30 pb or 35 bp in length, in view of modifying the expression of said genomic locus. Said expression can be modified by mutation, deletion or insertion into coding or regulatory polynucleotide sequences, by epigenetic change, such as by methylation or histone modification, or by interfering at the transcriptional level by interacting with transcription factors or polymerases.

Examples of sequence-specific reagents are endonucleases, RNA guides, RNAi, methylases, exonucleases, histone deacetylases, end-processing enzymes such as exonucleases, and more particularly cytidine deaminases such as those coupled with the CRISPR/cas9 system to perform base editing (i.e. nucleotide substitution) without necessarily resorting to cleavage by nucleases as described for instance by Hess et al. (Mol Cell. (2017) 68 (1): 26-43) and Rees et al. (Nat. Rev. Genet. (2018) 19, 770-788).

According to one aspect, at least 50%, at least 70%, at least 90%, or at least 95% of the cell population express a short hairpin RNA (shRNA) or small interfering (siRNA) directed against a polynucleotide sequence encoding a component of the TCR.

According to one aspect, at least 50%, at least 70%, at least 90%, or at least 95% of the cell population express a short hairpin RNA (shRNA) or small interfering (siRNA) directed against a polynucleotide sequence encoding β2M.

According to one aspect, at least 50%, at least 70%, at least 90%, or at least 95% of the cell population express a short hairpin RNA (shRNA) or small interfering (siRNA) directed against a polynucleotide sequence encoding CD52.

According to one aspect, at least 50%, at least 70%, at least 90%, or at least 95% of the cell population express a short hairpin RNA (shRNA) or small interfering (siRNA) directed against a polynucleotide sequence encoding PDCD1. According to one aspect, at least 50%, at least 70%, at least 90%, or at least 95% of the cell population express a short hairpin RNA (shRNA) or small interfering (siRNA) directed against a polynucleotide sequence encoding LAG3.

According to one aspect, at least 50%, at least 70%, at least 90%, or at least 95% of the cell population express a short hairpin RNA (shRNA) or small interfering (siRNA) directed against a polynucleotide sequence encoding TIM3.

According to one aspect, at least 50%, at least 70%, at least 90%, or at least 95% of the cell population express a short hairpin RNA (shRNA) or small interfering (siRNA) directed against a polynucleotide sequence encoding GM-CSF.

According to another aspect, at least 50%, at least 70%, at least 90%, or at least 95% of the cell population express a short hairpin RNA (shRNA) or small interfering (siRNA) directed against a polynucleotide sequence encoding a component of the TCR, as well as a short hairpin RNA (shRNA) or small interfering (siRNA) directed against a polynucleotide sequence encoding β2M and/or a short hairpin RNA (shRNA) or small interfering (siRNA) directed against a polynucleotide sequence encoding CD3.

In some cases, the sequence-specific reagent can be a sequence-specific nuclease reagent, such as a sequence-specific endonuclease like a rare-cutting endonuclease like TALE Nuclease, or a RNA guide coupled with a guided endonuclease like CRISPR.

The terms “sequence-specific nuclease reagent” include reagents that have nickase or endonuclease activity. The sequence-specific nuclease reagent can be a chimeric polypeptide comprising a DNA binding domain and another domain displaying catalytic activity. Such catalytic activity can be for instance a nuclease to perform gene inactivation, or nickase or double nickase to preferentially perform gene insertion by creating cohesive ends to facilitate gene integration by homologous recombination.

The term “endonuclease” generally refers to any wild-type or variant enzyme capable of catalyzing the hydrolysis (cleavage) of bonds between nucleic acids within a DNA or RNA molecule, a DNA molecule. Endonucleases (and, thus, sequence-specific endonucleases) do not cleave the DNA or RNA molecule irrespective of its sequence but recognize and cleave the DNA or RNA molecule at specific polynucleotide sequences, further referred to as “target sequences” or “target sites”. Endonucleases can be classified as rare-cutting endonucleases when having typically a polynucleotide recognition site greater than 10 base pairs (bp) in length, or of 14-55 bp. Rare-cutting endonucleases significantly increase homologous recombination by inducing DNA double-strand breaks (DSBs) at a defined locus thereby allowing gene repair or gene insertion therapies (Pingoud and Silva (2007) Nat. Biotechnol. 25 (7): 743-4).

In some cases, the sequence specific-reagent can be a base editor able to perform base editing as described for instance in Komor et al. (Nature (2019) 533 (7603), 420-424) and in Mok et al. (Nature (2020) 583:631-637).

The term “base editor”, as used herein, refers to a catalytic domain capable of making a modification to a base (e.g. A, T, C, G, or U) within a nucleic acid sequence by converting one base to another (e.g. A to G, A to C, A to T, C to T C to G, C to A, G to A, G to C, G to T, T to A, T to C, T to G). Base editors can include cytidine deaminases that convert target C/G to T/A and adenine base editors that convert target A/T to G/C. Adenosine deaminase can be, for instance, TadA or its variant TadA7.10 as described by Jeong et al. (Nat Biotechnol (2021) 39, 1426-1433). Different members of Apolipoprotein B mRNA editing enzyme (APOBEC) family can be used to convert cytidines to thymidines, such as the murine rAPOBEC1 and the human APOBEC3G as developed by Lee et al. (Science Advances (2020) 6 (29)).

In some cases, base editor catalytic domain can convert C to T (cytidine deaminase) and catalyzes the chemical reaction “cytosine+H2O→uracil+NH3” or “5-methyl-cytosine+H2O→thymine+NH3.” As it may be apparent from the reaction formula, such chemical reactions result in a C to U/T nucleobase change. In the context of a gene, such a nucleotide change, or mutation, may in turn lead to an amino acid change in the protein, which may affect the protein's function, e.g. loss-of-function or gain-of-function.

The sequence specific-reagents as defined herewith include TALE-base editors (BE), which can be generated by the fusion of transcription activator-like effector array proteins (TALE) with a base editor catalytic domain. The base editor catalytic domain can be a double-stranded DNA deaminase (“DddA”) that precisely makes nucleotide changes and/or corrects pathogenic mutations, rather than destroying DNA by double-strand breaks (DSBs). For instance, Mok et al. (Nature (2020) 583:631-637) recently developed TALE base editor by using the bacterial cytidine deaminase toxin DddAtox, from Burkholderia cenocepacia, that has been split into non-toxic halves which have been fused to the C-terminus of paired (left and right) TALE binding domains, respectively, to form heterodimeric TALE base editors. In such setting, the deaminase DddAtox becomes active when its two halves, linked to their respective TALE binding domains, co-localize at a predetermined genomic locus. The split “DddA-N half” and “DddA-C half” can be obtained by cleaving the full DddAtox protein (SEQ ID NO: 95) at positions 1333 or 1397.

In some cases, such TALE-base editors can also comprise a domain that inhibits uracil glycosylase referred to as “UGI”, and/or a nuclear localization signal. The term “uracil glycosylase inhibitor” or “UGI,” as used herein, refers to a protein that is capable of inhibiting an uracil-DNA glycosylase base-excision repair enzyme. In some cases, a UGI domain can comprise a wild-type UGI or a canonical UGI. In some cases, the UGI proteins can include fragments of UGI and proteins homologous to a UGI or a UGI fragment, which are useful to improve the specificity of base editing performed at a predetermined locus.

The methods and material provided herein aim to improve the therapeutic potential of immune cells through gene editing techniques, especially by gene targeted integration.

In some cases, an exogenous nucleic acid sequence encoding a chimeric antigen receptor (CAR) targeting a Fibroblast Activation Protein (FAP) (“FAP-CAR”) placed under the transcriptional control of an exogenous or endogenous constitutive promoter; and an exogenous nucleic acid sequence encoding a chimeric antigen receptor (CAR) targeting a tumor antigen (“tumor-CAR”) placed under the transcriptional control of an exogenous or endogenous inducible promoter can be integrated in the cells' genome.

Each of the exogenous nucleic acid sequence encoding a FAP-CAR and the exogenous nucleic acid sequence encoding a tumor-CAR as described herewith can, independently, be integrated in the cell's genome through random integration (such as through lentiviral vector integration) or through gene targeting integration (such as through sequence-specific endonuclease-mediated cDNA insertion at a targeted gene locus in the cells' genome).

In one instance, the exogenous nucleic acid sequence encoding a FAP-CAR and the exogenous nucleic acid sequence encoding a tumor-CAR, as described herewith, are integrated in the cell's genome through random integration (such as through lentiviral vector integration).

In another instance, the exogenous nucleic acid sequence encoding a FAP-CAR as described herewith is integrated in the cell's genome through random integration (such as through lentiviral vector integration) and the exogenous nucleic acid sequence encoding a tumor-CAR as described herewith is integrated in the cell's genome through gene targeting integration (such as through sequence-specific endonuclease-mediated cDNA insertion at a targeted gene locus in the cells' genome).

In a still other instance, the exogenous nucleic acid sequence encoding a FAP-CAR as described herewith is integrated in the cell's genome through gene targeting integration (such as through sequence-specific endonuclease-mediated cDNA insertion at a targeted gene locus in the cells' genome) and the exogenous nucleic acid sequence encoding a tumor-CAR as described herewith is integrated in the cell's genome through random integration (such as through lentiviral vector integration).

In a further instance, the exogenous nucleic acid sequence encoding a FAP-CAR and the exogenous nucleic acid sequence encoding a tumor-CAR, as described herewith, are integrated in the cell's genome through gene targeting integration (such as through sequence-specific endonuclease-mediated cDNA insertion at targeted gene loci in the cells' genome).

By “gene targeting integration” is meant any known site-specific methods allowing to insert, replace or correct a genomic coding sequence into a living cell.

In some cases, the gene targeted integration involves homologous gene recombination at the locus of the targeted gene to result in the insertion of, or replacement of the targeted gene by, at least one exogenous nucleotide sequence, such as a sequence of several nucleotides (i.e. polynucleotide), e.g. a coding sequence.

By “DNA target,” “DNA target sequence,” “target DNA sequence,” “nucleic acid target sequence,” “target sequence,” or “processing site” is intended a polynucleotide sequence that can be targeted and processed by a sequence-specific nuclease reagent as described herewith. These terms refer to a specific DNA location, such as a genomic location in a cell, but also to a portion of genetic material that can exist independently to the main body of genetic material such as plasmids, episomes, virus, transposons or in organelles such as mitochondria as non-limiting example. As non-limiting examples of RNA guided target sequences, are those genome sequences that can hybridize the guide RNA which directs the RNA guided endonuclease to a desired locus.

“Rare-cutting endonucleases” are sequence-specific endonuclease reagents of choice, insofar as their recognition sequences generally range from 10 to 50 successive base pairs, for instance from 12 to 30 bp or from 14 to 20 bp.

In some cases, said sequence-specific endonuclease reagent can be a nucleic acid encoding an “engineered” or “programmable” rare-cutting endonuclease, such as a homing endonuclease as described for instance by Arnould et al. (WO2004067736), a zinc finger nuclease (ZFN) as described, for instance, by Urnov et al. (Nature (2005) 435:646-651), a TALE-Nuclease as described, for instance, by Mussolino et al. (Nucl. Acids Res. (2011) 39 (21): 9283-9293), or a MegaTAL nuclease as described, for instance by Boissel et al. (Nucleic Acids Research (2013) 42 (4): 2591-2601).

In some cases, the endonuclease reagent can be a RNA-guide to be used in conjunction with a RNA guided endonuclease, such as Cas9 or Cpf1, as per, inter alia, the teaching by Doudna and Charpentier (Science (2014) 346 (6213): 1077), which is incorporated herein by reference.

In some cases, the endonuclease reagent can be transiently expressed into the cells, meaning that said reagent is not supposed to integrate into the genome or persist over a long period of time, such as would be the case of RNA, such as mRNA, proteins or complexes mixing proteins and nucleic acids (e.g. ribonucleoproteins).

An endonuclease under mRNA form can be synthetized with a cap to enhance its stability according to techniques well known in the art, as described, for instance, by Kore et al. (J Am Chem Soc. (2009) 131 (18): 6364-5).

The nucleases, polynucleotides encoding these nucleases, donor polynucleotides and compositions comprising the proteins and/or polynucleotides described herein for genetically modifying the cells may be delivered in vivo or ex vivo by any suitable means.

In some cases, polypeptides may be synthesized in situ in a cell as a result of the introduction of polynucleotides encoding the polypeptides into the cell. In some cases, the polypeptides can be produced outside the cell and then introduced into the cell. Methods for introducing a polynucleotide construct into cells are known in the art and include, as non-limiting examples, stable transformation methods wherein the polynucleotide construct is integrated into the genome of the cell, transient transformation methods wherein the polynucleotide construct is not integrated into the genome of the cell and virus mediated methods. In some cases, the polynucleotides can be introduced into a cell by recombinant viral vectors (e.g. retroviruses, adenoviruses), liposomes and the like. For example, transient transformation methods include, for example microinjection, electroporation or particle bombardment. The polynucleotides can be included in vectors, such as plasmids or virus, in view of being expressed in cells.

In some cases, the cells can be transfected with a nucleic acid encoding an endonuclease reagent. In some cases, 80% of the endonuclease reagent is degraded by 30 hours, for instance by 24 or by 20 hours after transfection.

In some cases, nucleases and/or donor constructs as described herein can also be delivered using vectors containing sequences encoding one or more of the CRISPR/Cas system(s), zinc finger or TALEN protein(s).

Any vector systems may be used including, but not limited to, plasmid vectors, retroviral vectors, lentiviral vectors, adenovirus vectors, poxvirus vectors; herpesvirus vectors and adeno-associated virus vectors, etc. See, also, U.S. Pat. Nos. 6,534,261; 6,607,882; 6,824,978; 6,933,113; 6,979,539; 7,013,219; and 7,163,824, incorporated by reference herein in their entireties. Furthermore, it will be apparent that any of these vectors may comprise one or more of the sequences needed for treatment. Thus, when one or more nucleases and a donor construct are introduced into the cell, the nucleases and/or donor polynucleotide may be carried on the same vector or on different vectors. When multiple vectors are used, each vector may comprise a sequence encoding one or multiple nucleases and/or donor constructs.

Any appropriate viral and non-viral based gene transfer methods can be used to introduce nucleic acids encoding nucleases and donor constructs in cells (e.g. mammalian cells) and target tissues.

Viral vector delivery systems include DNA and RNA viruses, which have either episomal or integrated genomes after delivery to the cell. For a review of gene therapy procedures, see Anderson, Science 256:808-813 (1992); Nabel & Feigner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6 (10): 1149-1154 (1988); Vigne, Restorative Neurology and Neuroscience 8:35-36 (1995); Kremer & Perricaudet, British Medical Bulletin 51 (1): 31-44 (1995); Haddada et al., in Current Topics in Microbiology and Immunology, Doerfler and Bohm (eds.) (1995); and Yu et al., Gene Therapy 1:13-26 (1994).

In some cases, methods of non-viral delivery of nucleic acids include electroporation, lipofection, microinjection, biolistics, virosomes, liposomes, immunoliposomes, polycation or lipid: nucleic acid conjugates, naked DNA, naked RNA, capped RNA, artificial virions, and agent-enhanced uptake of DNA. Sonoporation using, e.g. the Sonitron 2000 system (Rich-Mar) can also be used for delivery of nucleic acids.

In general, electroporation steps that are used to transfect primary immune cells, such as PBMCs are typically performed in closed chambers comprising parallel plate electrodes producing a pulse electric field between said parallel plate electrodes greater than 100 volts/cm and less than 5,000 volts/cm, substantially uniform throughout the treatment volume such as described in WO 2004/083379, which is incorporated by reference, especially from page 23, line 25 to page 29, line 11. One such electroporation chamber can have a geometric factor (cm⁻¹) defined by the quotient of the electrode gap squared (cm2) divided by the chamber volume (cm³), wherein the geometric factor is less than or equal to 0.1 cm⁻¹, wherein the suspension of the cells and the sequence-specific reagent is in a medium which is adjusted such that the medium has conductivity in a range spanning 0.01 to 1.0 milliSiemens. In general, the suspension of cells undergoes one or more pulsed electric fields. With the method, the treatment volume of the suspension is scalable, and the time of treatment of the cells in the chamber is substantially uniform.

In some cases, different transgenes or multiple copies of the transgene can be included in one vector. The vector can comprise a nucleic acid sequence encoding ribosomal skip sequence such as a sequence encoding a 2A peptide. 2A peptides, which were identified in the Aphthovirus subgroup of picornaviruses, causes a ribosomal “skip” from one codon to the next without the formation of a peptide bond between the two amino acids encoded by the codons (see Donnelly et al., J. of General Virology 82:1013-1025 (2001); Donnelly et al., J. of Gen. Virology 78:13-21 (1997); Doronina et al., Mol. And. Cell. Biology 28 (13): 4227-4239 (2008); Atkins et al., RNA 13:803-810 (2007)).

By “codon” is meant three nucleotides on an mRNA (or on the sense strand of a DNA molecule) that are translated by a ribosome into one amino acid residue. Thus, two polypeptides can be synthesized from a single, contiguous open reading frame within an mRNA when the polypeptides are separated by a 2A oligopeptide sequence that is in frame. Such ribosomal skip mechanisms are well known in the art and are known to be used by several vectors for the expression of several proteins encoded by a single messenger RNA.

In some cases, a polynucleotide encoding a sequence-specific reagent can be mRNA which is introduced directly into the cells, for example by electroporation. In some cases, the cells can be electroporated using cytoPulse technology which allows, by the use of pulsed electric fields, to transiently permeabilize living cells for delivery of material into the cells. The technology, based on the use of PulseAgile (BTX Havard Apparatus, 84 October Hill Road, Holliston, Mass. 01746, USA) electroporation waveforms grants the precise control of pulse duration, intensity as well as the interval between pulses (see U.S. Pat. No. 6,010,613 and published International Application WO 2004/083379). All these parameters can be modified in order to reach the best conditions for high transfection efficiency with minimal mortality. The first high electric field pulses allow pore formation, while subsequent lower electric field pulses allow moving the polynucleotide into the cell.

Additional exemplary nucleic acid delivery systems include those provided by Amaxa Biosystems (Cologne, Germany), Maxcyte, Inc. (Rockville, Md.), BTX Molecular Delivery Systems (Holliston, Mass.) and Copernicus Therapeutics Inc., (see for example U.S. Pat. No. 6,008,336). Lipofection is described in e.g. U.S. Pat. Nos. 5,049,386; 4,946,787; and 4,897,355) and lipofection reagents are sold commercially (e.g. Transfectam and Lipofectin). Cationic and neutral lipids that are suitable for efficient receptor-recognition lipofection of polynucleotides include those of Felgner, WO 91/17424, WO 91/16024.

The preparation of lipid: nucleic acid complexes, including targeted liposomes such as immunolipid complexes, is well known to one of skill in the art (see, e.g. Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

In some cases, the donor sequence and/or sequence-specific reagent can be encoded by a viral vector. In some cases, adenoviral based systems can be used. Adenoviral based vectors are capable of very high transduction efficiency in many cell types and do not require cell division. With such vectors, high titer and high levels of expression have been obtained. This vector can be produced in large quantities in a relatively simple system. Adeno-associated virus (“AAV”) vectors are also used to transduce cells with target nucleic acids, e.g. in the in vitro production of nucleic acids and peptides, and for in vivo and ex vivo gene therapy procedures (see, e.g. West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989).

Recombinant adeno-associated virus vectors (rAAV) are a promising alternative gene delivery system based on the defective and nonpathogenic parvovirus adeno-associated type 2 virus. All vectors are derived from a plasmid that retains only the AAV 145 bp inverted terminal repeats flanking the transgene expression cassette. Efficient gene transfer and stable transgene delivery due to integration into the genomes of the transduced cell are key features for this vector system (Wagner et al., Lancet 351:9117 1702-3 (1998), Kearns et al., Gene Ther. 9:748-55 (1996)). Other AAV serotypes, including but non-limiting example, AAV1, AAV3, AAV4, AAV5, AAV6, AAV8, AAV 8.2, AAV9, and AAV rh10 and pseudotyped AAV such as AAV2/8, AAV2/5 and AAV2/6 can also be used in accordance with the present disclosure.

In some cases, the cells can be administered with an effective amount of one or more caspase inhibitors in combination with an AAV vector.

In some cases, the donor sequence and/or sequence-specific reagent can be encoded by a recombinant lentiviral vector (rLV).

The nuclease-encoding sequences and donor constructs can be delivered using the same or different systems. For example, a donor polynucleotide can be carried by a viral vector, while the one or more nucleases can be delivered as mRNA compositions.

In some cases, one or more reagents can be delivered to cells using nanoparticles. In some cases, nanoparticles are coated with ligands, such as antibodies, having a specific affinity towards HSC surface proteins, such as CD105 (Uniprot #P17813). In some cases, the nanoparticles are biodegradable polymeric nanoparticles in which the sequence-specific reagents under polynucleotide form are complexed with a polymer of polybeta amino ester and coated with polyglutamic acid (PGA).

Due to their higher specificity, TALE-nuclease have proven to be particularly appropriate sequence-specific nuclease reagents for therapeutic applications, especially under heterodimeric forms—i.e. working by pairs with a “right” monomer (also referred to as “5′” or “forward”) and ‘left” monomer (also referred to as “3′” or “reverse”) as reported for instance by Mussolino et al. (Nucl. Acids Res. (2014) 42 (10): 6762-6773).

As previously stated, the sequence-specific reagent can be under the form of nucleic acids, such as under DNA or RNA form encoding a rare cutting endonuclease or a subunit thereof, but they can also be part of conjugates involving polynucleotide(s) and polypeptide(s) such as so-called “ribonucleoproteins.” Such conjugates can be formed with reagents as Cas9 or Cpf1 (RNA-guided endonucleases) as respectively described by Zetsche et al. (Cell (2015) 163 (3): 759-771), which involve RNA or DNA guides that can be complexed with their respective nucleases.

“Exogenous sequence” refers to any nucleotide or nucleic acid sequence that was not initially present at the selected locus. This sequence may be homologous to, or a copy of, a genomic sequence, or be a foreign sequence introduced into the cell. By opposition, “endogenous sequence” means a cell genomic sequence initially present at a locus.

As used herewith, a “donor construct” or “donor polynucleotide” comprises the exogenous nucleotide sequence to be inserted in the cell's genome either randomly at any locus or at, or replacing, the targeted gene locus. A donor construct can comprise a nucleotide sequence encoding a CAR described herewith and, optionally, a promoter controlling the transcription of said CAR.

In some cases, the donor construct can be a vector comprising a constitutive exogenous promoter and, operably linked to said promoter, an exogenous nucleic acid sequence encoding a FAP-CAR, as described herewith. In this case, the donor construct can be integrated in the cell's genome randomly at any locus and the FAP-CAR transcription is controlled by said constitutive exogenous promoter.

In some cases, the donor construct can be a vector comprising an exogenous nucleic acid sequence encoding a FAP-CAR flanked by Left- and Right-Homologous Arms (or “Left- and Right-Homologous Regions”) (also called “5′- and 3′-Homology Arms (or Regions)”, respectively) which have homology to the targeted gene locus, the expression of which is constitutive as described herewith. In some cases, the vector does not comprise a promoter sequence and the donor construct can be integrated in the cell's genome by homologous recombination at the targeted constitutively expressed gene locus so that the FAP-CAR transcription is controlled by the constitutive (endogenous) promoter of said targeted gene locus. In some cases, the vector further comprises a constitutive exogenous promoter sequence and the “cassette” comprising the promoter sequence and the exogenous nucleic acid sequence encoding a FAP-CAR is flanked by Left- and Right-Homologous Arms which have homology to the targeted gene locus. In these later cases, the donor construct can be integrated in the cell's genome by homologous recombination at the targeted gene locus but the FAP-CAR transcription is controlled by the constitutive exogenous promoter provided by the vector.

In some cases, the donor construct can be a vector comprising an exogenous inducible promoter and an exogenous nucleic acid sequence encoding a tumor-CAR, as described herewith. In this case, the donor construct can be integrated in the cell's genome randomly at any locus and the tumor-CAR transcription is controlled by said exogenous inducible promoter.

In some cases, the donor construct can be a vector comprising an exogenous nucleic acid sequence encoding a tumor-CAR flanked by Left- and Right-Homologous Arms which have homology to the targeted gene locus. In some cases, the vector does not comprise a promoter sequence and the donor construct can be integrated in the cell's genome by homologous recombination at the targeted inducible gene locus so that the tumor-CAR transcription is controlled by the inducible (endogenous) promoter of said targeted gene locus. In some cases, the vector further comprises an inducible exogenous promoter sequence and the “cassette” comprising the promoter sequence and the exogenous nucleic acid sequence encoding a tumor-CAR is flanked by Left- and Right-Homologous Arms which have homology to the targeted gene locus. In these later some cases, the donor construct can be integrated in the cell's genome by homologous recombination at the targeted gene locus but the tumor-CAR transcription is controlled by the inducible exogenous promoter provided by the vector.

When the donor construct does not comprise a promoter, the donor construct can comprise, in addition to the CAR coding sequence, an Internal Ribosome Entry Site (IRES) or “self-cleaving” 2A peptides, such as T2A, P2A, E2A or F2A, so that to allow production of a functional CAR protein.

Stable expression of CARs, in particular the FAP-CAR and tumor-CAR described herewith, in the above-described immune cells, such as T-cells, can be achieved using, for example, viral vectors (e.g. lentiviral vectors, retroviral vectors, Adeno-Associated Virus (AAV) vectors) or transposon/transposase systems or plasmids or PCR products integration. Other approaches include direct mRNA electroporation.

In some cases, the tumor-CAR and the FAP-CAR have the same hinge, transmembrane domain, and/or cytoplasmic domain. In these cases, to avoid any recombination event within a construct comprising polynucleotides encoding two CARs comprising identical domains, the nucleotide acid sequences used to code for the same amino acid sequences present twice in the construct (e.g. the same hinge, transmembrane domain, or the same stimulatory domain) are optimized using codon usage and code degeneracy so that the nucleotide sequences diverge.

Non-limitative examples of TALE-nuclease targeting the endogenous genes expressing PDCD1, TRAC, CD52, and B2M are provided in Table 6. The invention can be practiced as described herein with such polynucleotides or polypeptides having at least 70%, for instance at least 80%, at least 90% or at least 95% or 99% identity with the sequences referred to in Table 6.

TABLE 6
Examples of TALE-nucleases and their target sequences

| Targeted | SEQ | |
|---|---|---|
| gene | ID # | Target sequence |
| PDCD1_T01- | 98 | TACCTCTGTGGGGCCATctccctggcccccaaGGCGCAGA |
| target | | TCAAAGAGA |
| TRAC_T01- | 99 | TTGTCCCACAGATATCCagaaccctgaccctgCCGTGTAC |
| target | | CAGCTGAGA |
| CD52_T02- | 100 | TTCCTCCTACTCACCATcagcctcctggttatGGTACAGG |
| target | | TAAGAGCAA |
| B2M_T02- | 101 | TTAGCTGTGCTCGCGCTactctctctttctGGCCTGGAGG |
| target | | CTATCCA |
| CIITA_T01- | 102 | TCTGGCTGGGCTGATCTTCCAGCCTCCcgcccgctgcctg |
| target | | ggAGCCCTA |
| CS1_target | 103 | TATATCCTTTGGCAGCTcacaggtgagtccGGCCGGATTC |
| | | TCTTCCA |
| TALE- | | |
| Nuclease | SEQ | |
| monomer | ID # | TALE-Nuclease monomer sequence |
| PDCD1_T01-L | 72 | MGDPKKKRKVIDIADLRTLGYSQQQQEKIKPKVRSTVAQH |
| | | HEALVGHGFTHAHIVALSQHPAALGTVAVKYQDMIAALPE |
| | | ATHEAIVGVGKQWSGARALEALLTVAGELRGPPLQLDTGQ |
| | | LLKIAKRGGVTAVEAVHAWRNALTGAPLNLTPEQVVAIAS |
| | | HDGGKQALETVQRLLPVLCQAHGLTPQQVVAIASNGGGKQ |
| | | ALETVQRLLPVLCQAHGLTPEQVVAIASHDGGKQALETVQ |
| | | RLLPVLCQAHGLTPQQVVAIASNGGGKQALETVQRLLPVL |
| | | CQAHGLTPQQVVAIASNGGGKQALETVQRLLPVLCQAHGL |
| | | TPQQVVAIASNGGGKQALETVQRLLPVLCQAHGLTPQQVV |
| | | AIASNNGGKQALETVQRLLPVLCQAHGLTPEQVVAIASNI |
| | | GGKQALETVQALLPVLCQAHGLTPQQVVAIASNGGGKQAL |
| | | ETVQRLLPVLCQAHGLTPEQVVAIASHDGGKQALETVQRL |
| | | LPVLCQAHGLTPQQVVAIASNGGGKQALETVQRLLPVLCQ |
| | | AHGLTPQQVVAIASNNGGKQALETVQRLLPVLCQAHGLTP |
| | | EQVVAIASNGGKQALETVQRLLPVLCQAHGLTPQQVVAIA |
| | | SNNGGKQALETVQRLLPVLCQAHGLTPEQVVAIASHDGGK |
| | | QALETVQRLLPVLCQAHGLTPQQVVAIASNGGGRPALESI |
| | | VAQLSRPDPALAALTNDHLVALACLGGRPALDAVKKGLGD |
| | | PISRSQLVKSELEEKKSELRHKLKYVPHEYIELIEIARNS |
| | | TQDRILEMKVMEFFMKVYGYRGKHLGGSRKPDGAIYTVGS |
| | | PIDYGVIVDTKAYSGGYNLPIGQADEMQRYVEENQTRNKH |
| | | INPNEWWKVYPSSVTEFKFLFVSGHFKGNYKAQLTRLNHI |
| | | TNCNGAVLSVEELLIGGEMIKAGTLTLEEVRRKENNGEIN |
| | | FAAD |
| PDCD1_T01-R | 73 | MGDPKKKRKVIDIADLRTLGYSQQQQEKIKPKVRSTVAQH |
| | | HEALVGHGFTHAHIVALSQHPAALGTVAVKYQDMIAALPE |
| | | ATHEAIVGVGKQWSGARALEALLTVAGELRGPPLQLDTGQ |
| | | LLKIAKRGGVTAVEAVHAWRNALTGAPLNLTPEQVVAIAS |
| | | KLGGKQALETVQALLPVLCQAHGLTPEQVVAIASHDGGKQ |
| | | ALETVQRLLPVLCQAHGLTPEQVVAIASHDGGKQALETVQ |
| | | RLLPVLCQAHGLTPQQVVAIASNGGGKQALETVQRLLPVL |
| | | CQAHGLTPEQVVAIASHDGGKQALETVQRLLPVLCQAHGL |
| | | TPQQVVAIASNGGGKQALETVQRLLPVLCQAHGLTPQQVV |
| | | AIASYKGGKQALETVQRLLPVLCQAHGLTPQQVVAIASNG |
| | | GGKQALETVQRLLPVLCQAHGLTPQQVVAIASNNGGKQAL |
| | | ETVQRLLPVLCQAHGLTPQQVVAIASNNGGKQALETVQRL |
| | | LPVLCQAHGLTPQQVVAIASNNGGKQALETVQRLLPVLCQ |
| | | AHGLTPQQVVAIASNNGGKQALETVQRLLPVLCQAHGLTP |
| | | EQVVAIASHDGGKQALETVQRLLPVLCQAHGLTPEQVVAI |
| | | ASHDGGKQALETVQRLLPVLCQAHGLTPEQVVAIASNIGG |
| | | KQALETVQALLPVLCQAHGLTPQQVVAIASNGGGRPALES |
| | | IVAQLSRPDPALAALTNDHLVALACLGGRPALDAVKKGLG |
| | | DPISRSQLVKSELEEKKSELRHKLKYVPHEYIELIEIARN |
| | | STQDRILEMKVMEFFMKVYGYRGKHLGGSRKPDGAIYTVG |
| | | SPIDYGVIVDTKAYSGGYNLPIGQADEMQRYVEENQTRNK |
| | | HINPNEWWKVYPSSVTEFKFLFVSGHFKGNYKAQLTRLNH |
| | | ITNCNGAVLSVEELLIGGEMIKAGTLTLEEVRRKENNGEI |
| | | NFAAD |
| TRAC_T01-L | 74 | MGDPKKKRKVIDIADLRTLGYSQQQQEKIKPKVRSTVAQH |
| | | HEALVGHGFTHAHIVALSQHPAALGTVAVKYQDMIAALPE |
| | | ATHEAIVGVGKQWSGARALEALLTVAGELRGPPLQLDTGQ |
| | | LLKIAKRGGVTAVEAVHAWRNALTGAPLNLTPQQVVAIAS |
| | | NGGGKQALETVQRLLPVLCQAHGLTPQQVVAIASNNGGKQ |
| | | ALETVQRLLPVLCQAHGLTPQQVVAIASNGGGKQALETVQ |
| | | RLLPVLCQAHGLTPEQVVAIASHDGGKQALETVQRLLPVL |
| | | CQAHGLTPEQVVAIASHDGGKQALETVQRLLPVLCQAHGL |
| | | TPEQVVAIASHDGGKQALETVQRLLPVLCQAHGLTPEQVV |
| | | AIASNIGGKQALETVQALLPVLCQAHGLTPEQVVAIASHD |
| | | GGKQALETVQRLLPVLCQAHGLTPEQVVAIASNIGGKQAL |
| | | ETVQALLPVLCQAHGLTPQQVVAIASNNGGKQALETVQRL |
| | | LPVLCQAHGLTPEQVVAIASNIGGKQALETVQALLPVLCQ |
| | | AHGLTPQQVVAIASNGGGKQALETVQRLLPVLCQAHGLTP |
| | | EQVVAIASNIGGKQALETVQALLPVLCQAHGLTPQQVVAI |
| | | ASNGGGKQALETVQRLLPVLCQAHGLTPEQVVAIASHDGG |
| | | KQALETVQRLLPVLCQAHGLTPQQVVAIASNGGGRPALES |
| | | IVAQLSRPDPALAALTNDHLVALACLGGRPALDAVKKGLG |
| | | DPISRSQLVKSELEEKKSELRHKLKYVPHEYIELIEIARN |
| | | STQDRILEMKVMEFFMKVYGYRGKHLGGSRKPDGAIYTVG |
| | | SPIDYGVIVDTKAYSGGYNLPIGQADEMQRYVEENQTRNK |
| | | HINPNEWWKVYPSSVTEFKFLFVSGHFKGNYKAQLTRLNH |
| | | ITNCNGAVLSVEELLIGGEMIKAGTLTLEEVRRKENNGEI |
| | | NFAAD |
| TRAC_T01-R | 75 | MGDPKKKRKVIDIADLRTLGYSQQQQEKIKPKVRSTVAQH |
| | | HEALVGHGFTHAHIVALSQHPAALGTVAVKYQDMIAALPE |
| | | ATHEAIVGVGKQWSGARALEALLTVAGELRGPPLQLDTGQ |
| | | LLKIAKRGGVTAVEAVHAWRNALTGAPLNLTPEQVVAIAS |
| | | HDGGKQALETVQRLLPVLCQAHGLTPQQVVAIASNGGGKQ |
| | | ALETVQRLLPVLCQAHGLTPEQVVAIASHDGGKQALETVQ |
| | | RLLPVLCQAHGLTPEQVVAIASNIGGKQALETVQALLPVL |
| | | CQAHGLTPQQVVAIASNNGGKQALETVQRLLPVLCQAHGL |
| | | TPEQVVAIASHDGGKQALETVQRLLPVLCQAHGLTPQQVV |
| | | AIASNGGGKQALETVQRLLPVLCQAHGLTPQQVVAIASNN |
| | | GGKQALETVQRLLPVLCQAHGLTPQQVVAIASNNGGKQAL |
| | | ETVQRLLPVLCQAHGLTPQQVVAIASNGGGKQALETVQRL |
| | | LPVLCQAHGLTPEQVVAIASNIGGKQALETVQALLPVLCQ |
| | | AHGLTPEQVVAIASHDGGKQALETVQRLLPVLCQAHGLTP |
| | | EQVVAIASNIGGKQALETVQALLPVLCQAHGLTPEQVVAI |
| | | ASHDGGKQALETVQRLLPVLCQAHGLTPQQVVAIASNNGG |
| | | KQALETVQRLLPVLCQAHGLTPQQVVAIASNGGGRPALES |
| | | IVAQLSRPDPALAALTNDHLVALACLGGRPALDAVKKGLG |
| | | DPISRSQLVKSELEEKKSELRHKLKYVPHEYIELIEIARN |
| | | STQDRILEMKVMEFFMKVYGYRGKHLGGSRKPDGAIYTVG |
| | | SPIDYGVIVDTKAYSGGYNLPIGQADEMQRYVEENQTRNK |
| | | HINPNEWWKVYPSSVTEFKFLFVSGHFKGNYKAQLTRLNH |
| | | ITNCNGAVLSVEELLIGGEMIKAGTLTLEEVRRKENNGEI |
| | | NFAAD |
| CD52_T01-L | 76 | MGDPKKKRKVIDIADLRTLGYSQQQQEKIKPKVRSTVAQH |
| | | HEALVGHGFTHAHIVALSQHPAALGTVAVKYQDMIAALPE |
| | | ATHEAIVGVGKQWSGARALEALLTVAGELRGPPLQLDTGQ |
| | | LLKIAKRGGVTAVEAVHAWRNALTGAPLNLTPQQVVAIAS |
| | | NGGGKQALETVQRLLPVLCQAHGLTPEQVVAIASHDGGKQ |
| | | ALETVQRLLPVLCQAHGLTPEQVVAIASHDGGKQALETVQ |
| | | RLLPVLCQAHGLTPQQVVAIASNGGGKQALETVQRLLPVL |
| | | CQAHGLTPEQVVAIASHDGGKQALETVQRLLPVLCQAHGL |
| | | TPEQVVAIASHDGGKQALETVQRLLPVLCQAHGLTPQQVV |
| | | AIASNGGGKQALETVQRLLPVLCQAHGLTPEQVVAIASNI |
| | | GGKQALETVQALLPVLCQAHGLTPEQVVAIASHDGGKQAL |
| | | ETVQRLLPVLCQAHGLTPQQVVAIASNGGGKQALETVQRL |
| | | LPVLCQAHGLTPEQVVAIASHDGGKQALETVQRLLPVLCQ |
| | | AHGLTPEQVVAIASNIGGKQALETVQALLPVLCQAHGLTP |
| | | EQVVAIASHDGGKQALETVQRLLPVLCQAHGLTPEQVVAI |
| | | ASHDGGKQALETVQRLLPVLCQAHGLTPEQVVAIASNIGG |
| | | KQALETVQALLPVLCQAHGLTPQQVVAIASNGGGRPALES |
| | | IVAQLSRPDPALAALTNDHLVALACLGGRPALDAVKKGLG |
| | | DPISRSQLVKSELEEKKSELRHKLKYVPHEYIELIEIARN |
| | | STQDRILEMKVMEFFMKVYGYRGKHLGGSRKPDGAIYTVG |
| | | SPIDYGVIVDTKAYSGGYNLPIGQADEMQRYVEENQTRNK |
| | | HINPNEWWKVYPSSVTEFKFLFVSGHFKGNYKAQLTRLNH |
| | | ITNCNGAVLSVEELLIGGEMIKAGTLTLEEVRRKENNGEI |
| | | NFAAD |
| CD52_T01-R | 77 | MGDPKKKRKVIDIADLRTLGYSQQQQEKIKPKVRSTVAQH |
| | | HEALVGHGFTHAHIVALSQHPAALGTVAVKYQDMIAALPE |
| | | ATHEAIVGVGKQWSGARALEALLTVAGELRGPPLQLDTGQ |
| | | LLKIAKRGGVTAVEAVHAWRNALTGAPLNLTPQQVVAIAS |
| | | NGGGKQALETVQRLLPVLCQAHGLTPQQVVAIASNNGGKQ |
| | | ALETVQRLLPVLCQAHGLTPEQVVAIASHDGGKQALETVQ |
| | | RLLPVLCQAHGLTPQQVVAIASNGGGKQALETVQRLLPVL |
| | | CQAHGLTPEQVVAIASHDGGKQALETVQRLLPVLCQAHGL |
| | | TPQQVVAIASNGGGKQALETVQRLLPVLCQAHGLTPQQVV |
| | | AIASNGGGKQALETVQRLLPVLCQAHGLTPEQVVAIASNI |
| | | GGKQALETVQALLPVLCQAHGLTPEQVVAIASHDGGKQAL |
| | | ETVQRLLPVLCQAHGLTPEQVVAIASHDGGKQALETVQRL |
| | | LPVLCQAHGLTPQQVVAIASNGGGKQALETVQRLLPVLCQ |
| | | AHGLTPQQVVAIASNNGGKQALETVQRLLPVLCQAHGLTP |
| | | QQVVAIASNGGGKQALETVQRLLPVLCQAHGLTPEQVVAI |
| | | ASNIGGKQALETVQALLPVLCQAHGLTPEQVVAIASHDGG |
| | | KQALETVQRLLPVLCQAHGLTPQQVVAIASNGGGRPALES |
| | | IVAQLSRPDPALAALTNDHLVALACLGGRPALDAVKKGLG |
| | | DPISRSQLVKSELEEKKSELRHKLKYVPHEYIELIEIARN |
| | | STQDRILEMKVMEFFMKVYGYRGKHLGGSRKPDGAIYTVG |
| | | SPIDYGVIVDTKAYSGGYNLPIGQADEMQRYVEENQTRNK |
| | | HINPNEWWKVYPSSVTEFKFLFVSGHFKGNYKAQLTRLNH |
| | | ITNCNGAVLSVEELLIGGEMIKAGTLTLEEVRRKENNGEI |
| | | NFAAD |
| B2M_T01-L4 | 78 | MGDPKKKRKVIDIADLRTLGYSQQQQEKIKPKVRSTVAQH |
| | | HEALVGHGFTHAHIVALSQHPAALGTVAVKYQDMIAALPE |
| | | ATHEAIVGVGKQWSGARALEALLTVAGELRGPPLQLDTGQ |
| | | LLKIAKRGGVTAVEAVHAWRNALTGAPLNLTPQQVVAIAS |
| | | NGGGKQALETVQRLLPVLCQAHGLTPEQVVAIASNIGGKQ |
| | | ALETVQALLPVLCQAHGLTPQQVVAIASNNGGKQALETVQ |
| | | RLLPVLCQAHGLTPEQVVAIASHDGGKQALETVQRLLPVL |
| | | CQAHGLTPQQVVAIASNGGGKQALETVQRLLPVLCQAHGL |
| | | TPQQVVAIASNNGGKQALETVQRLLPVLCQAHGLTPQQVV |
| | | AIASNGGGKQALETVQRLLPVLCQAHGLTPQQVVAIASNN |
| | | GGKQALETVQRLLPVLCQAHGLTPEQVVAIASHDGGKQAL |
| | | ETVQRLLPVLCQAHGLTPQQVVAIASNGGGKQALETVQRL |
| | | LPVLCQAHGLTPEQVVAIASHDGGKQALETVQRLLPVLCQ |
| | | AHGLTPQQVVAIASNNGGKQALETVQRLLPVLCQAHGLTP |
| | | EQVVAIASHDGGKQALETVQRLLPVLCQAHGLTPQQVVAI |
| | | ASNNGGKQALETVQRLLPVLCQAHGLTPEQVVAIASHDGG |
| | | KQALETVQRLLPVLCQAHGLTPQQVVAIASNGGGRPALES |
| | | IVAQLSRPDPALAALTNDHLVALACLGGRPALDAVKKGLG |
| | | DPISRSQLVKSELEEKKSELRHKLKYVPHEYIELIEIARN |
| | | STQDRILEMKVMEFFMKVYGYRGKHLGGSRKPDGAIYTVG |
| | | SPIDYGVIVDTKAYSGGYNLPIGQADEMQRYVEENQTRNK |
| | | HINPNEWWKVYPSSVTEFKFLFVSGHFKGNYKAQLTRLNH |
| | | ITNCNGAVLSVEELLIGGEMIKAGTLTLEEVRRKENNGEI |
| | | NFAAD |
| B2M_T01-R4 | 79 | MGDPKKKRKVIDIADLRTLGYSQQQQEKIKPKVRSTVAQH |
| | | HEALVGHGFTHAHIVALSQHPAALGTVAVKYQDMIAALPE |
| | | ATHEAIVGVGKQWSGARALEALLTVAGELRGPPLQLDTGQ |
| | | LLKIAKRGGVTAVEAVHAWRNALTGAPLNLTPQQVVAIAS |
| | | NNGGKQALETVQRLLPVLCQAHGLTPQQVVAIASNNGGKQ |
| | | ALETVQRLLPVLCQAHGLTPEQVVAIASNIGGKQALETVQ |
| | | ALLPVLCQAHGLTPQQVVAIASNGGGKQALETVQRLLPVL |
| | | CQAHGLTPEQVVAIASNIGGKQALETVQALLPVLCQAHGL |
| | | TPQQVVAIASNNGGKQALETVQRLLPVLCQAHGLTPEQVV |
| | | AIASHDGGKQALETVQRLLPVLCQAHGLTPEQVVAIASHD |
| | | GGKQALETVQRLLPVLCQAHGLTPQQVVAIASNGGGKQAL |
| | | ETVQRLLPVLCQAHGLTPEQVVAIASHDGGKQALETVQRL |
| | | LPVLCQAHGLTPEQVVAIASHDGGKQALETVQRLLPVLCQ |
| | | AHGLTPEQVVAIASNIGGKQALETVQALLPVLCQAHGLTP |
| | | QQVVAIASNNGGKQALETVQRLLPVLCQAHGLTPQQVVAI |
| | | ASNNGGKQALETVQRLLPVLCQAHGLTPEQVVAIASHDGG |
| | | KQALETVQRLLPVLCQAHGLTPQQVVAIASNGGGRPALES |
| | | IVAQLSRPDPALAALTNDHLVALACLGGRPALDAVKKGLG |
| | | DPISRSQLVKSELEEKKSELRHKLKYVPHEYIELIEIARN |
| | | STQDRILEMKVMEFFMKVYGYRGKHLGGSRKPDGAIYTVG |
| | | SPIDYGVIVDTKAYSGGYNLPIGQADEMQRYVEENQTRNK |
| | | HINPNEWWKVYPSSVTEFKFLFVSGHFKGNYKAQLTRLNH |
| | | ITNCNGAVLSVEELLIGGEMIKAGTLTLEEVRRKFNNGEI |
| | | NFAAD |
| CIITA_T01-L | 80 | MGDPKKKRKVIDIADLRTLGYSQQQQEKIKPKVRSTVAQH |
| | | HEALVGHGFTHAHIVALSQHPAALGTVAVKYQDMIAALPE |
| | | ATHEAIVGVGKQWSGARALEALLTVAGELRGPPLQLDTGQ |
| | | LLKIAKRGGVTAVEAVHAWRNALTGAPLNLTPEQVVAIAS |
| | | HDGGKQALETVQRLLPVLCQAHGLTPQQVVAIASNGGGKQ |
| | | ALETVQRLLPVLCQAHGLTPQQVVAIASNNGGKQALETVQ |
| | | RLLPVLCQAHGLTPQQVVAIASNNGGKQALETVQRLLPVL |
| | | CQAHGLTPEQVVAIASHDGGKQALETVQRLLPVLCQAHGL |
| | | TPQQVVAIASNGGGKQALETVQRLLPVLCQAHGLTPQQVV |
| | | AIASNNGGKQALETVQRLLPVLCQAHGLTPQQVVAIASNN |
| | | GGKQALETVQRLLPVLCQAHGLTPQQVVAIASNNGGKQAL |
| | | ETVQRLLPVLCQAHGLTPEQVVAIASHDGGKQALETVQRL |
| | | LPVLCQAHGLTPQQVVAIASNGGGKQALETVQRLLPVLCQ |
| | | AHGLTPQQVVAIASNNGGKQALETVQRLLPVLCQAHGLTP |
| | | EQVVAIASNIGGKQALETVQALLPVLCQAHGLTPQQVVAI |
| | | ASNGGGKQALETVQRLLPVLCQAHGLTPEQVVAIASHDGG |
| | | KQALETVQRLLPVLCQAHGLTPQQVVAIASNGGGRPALES |
| | | IVAQLSRPDPALAALTNDHLVALACLGGRPALDAVKKGLG |
| | | DPISRSQLVKSELEEKKSELRHKLKYVPHEYIELIEIARN |
| | | STQDRILEMKVMEFFMKVYGYRGKHLGGSRKPDGAIYTVG |
| | | SPIDYGVIVDTKAYSGGYNLPIGQADEMQRYVEENQTRNK |
| | | HINPNEWWKVYPSSVTEFKFLFVSGHFKGNYKAQLTRLNH |
| | | ITNCNGAVLSVEELLIGGEMIKAGTLTLEEVRRKENNGEI |
| | | NFAAD |
| CIITA_T01-R | 81 | MGDPKKKRKVIDIADLRTLGYSQQQQEKIKPKVRSTVAQH |
| | | HEALVGHGFTHAHIVALSQHPAALGTVAVKYQDMIAALPE |
| | | ATHEAIVGVGKQWSGARALEALLTVAGELRGPPLQLDTGQ |
| | | LLKIAKRGGVTAVEAVHAWRNALTGAPLNLTPEQVVAIAS |
| | | NIGGKQALETVQALLPVLCQAHGLTPQQVVAIASNNGGKQ |
| | | ALETVQRLLPVLCQAHGLTPQQVVAIASNNGGKQALETVQ |
| | | RLLPVLCQAHGLTPQQVVAIASNNGGKQALETVQRLLPVL |
| | | CQAHGLTPEQVVAIASHDGGKQALETVQRLLPVLCQAHGL |
| | | TPQQVVAIASNGGGKQALETVQRLLPVLCQAHGLTPEQVV |
| | | AIASHDGGKQALETVQRLLPVLCQAHGLTPEQVVAIASHD |
| | | GGKQALETVQRLLPVLCQAHGLTPEQVVAIASHDGGKQAL |
| | | ETVQRLLPVLCQAHGLTPEQVVAIASNIGGKQALETVQAL |
| | | LPVLCQAHGLTPQQVVAIASNNGGKQALETVQRLLPVLCQ |
| | | AHGLTPQQVVAIASNNGGKQALETVQRLLPVLCQAHGLTP |
| | | EQVVAIASHDGGKQALETVQRLLPVLCQAHGLTPEQVVAI |
| | | ASNIGGKQALETVQALLPVLCQAHGLTPQQVVAIASNNGG |
| | | KQALETVQRLLPVLCQAHGLTPQQVVAIASNGGGRPALES |
| | | IVAQLSRPDPALAALTNDHLVALACLGGRPALDAVKKGLG |
| | | DPISRSQLVKSELEEKKSELRHKLKYVPHEYIELIEIARN |
| | | STQDRILEMKVMEFFMKVYGYRGKHLGGSRKPDGAIYTVG |
| | | SPIDYGVIVDTKAYSGGYNLPIGQADEMQRYVEENQTRNK |
| | | HINPNEWWKVYPSSVTEFKFLFVSGHFKGNYKAQLTRLNH |
| | | ITNCNGAVLSVEELLIGGEMIKAGTLTLEEVRRKFNNGEI |
| | | NFAAD |
| CS1_L | 82 | MGDPKKKRKVIDIADLRTLGYSQQQQEKIKPKVRSTVAQH |
| | | HEALVGHGFTHAHIVALSQHPAALGTVAVKYQDMIAALPE |
| | | ATHEAIVGVGKQWSGARALEALLTVAGELRGPPLQLDTGQ |
| | | LLKIAKRGGVTAVEAVHAWRNALTGAPLNLTPEQVVAIAS |
| | | NIGGKQALETVQALLPVLCQAHGLTPQQVVAIASNGGGKQ |
| | | ALETVQRLLPVLCQAHGLTPEQVVAIASNIGGKQALETVQ |
| | | ALLPVLCQAHGLTPQQVVAIASNGGGKQALETVQRLLPVL |
| | | CQAHGLTPEQVVAIASHDGGKQALETVQRLLPVLCQAHGL |
| | | TPEQVVAIASHDGGKQALETVQRLLPVLCQAHGLTPQQVV |
| | | AIASNGGGKQALETVQRLLPVLCQAHGLTPQQVVAIASNG |
| | | GGKQALETVQRLLPVLCQAHGLTPQQVVAIASNGGGKQAL |
| | | ETVQRLLPVLCQAHGLTPQQVVAIASNNGGKQALETVQRL |
| | | LPVLCQAHGLTPQQVVAIASNNGGKQALETVQRLLPVLCQ |
| | | AHGLTPEQVVAIASHDGGKQALETVQRLLPVLCQAHGLTP |
| | | EQVVAIASNIGGKQALETVQALLPVLCQAHGLTPQQVVAI |
| | | ASNNGGKQALETVQRLLPVLCQAHGLTPEQVVAIASHDGG |
| | | KQALETVQRLLPVLCQAHGLTPQQVVAIASNGGGRPALES |
| | | IVAQLSRPDPSGSGSGGDPISRSQLVKSELEEKKSELRHK |
| | | LKYVPHEYIELIEIARNSTQDRILEMKVMEFFMKVYGYRG |
| | | KHLGGSRKPDGAIYTVGSPIDYGVIVDTKAYSGGYNLPIG |
| | | QADEMQRYVEENQTRNKHINPNEWWKVYPSSVTEFKFLFV |
| | | SGHFKGNYKAQLTRLNHITNCNGAVLSVEELLIGGEMIKA |
| | | GTLTLEEVRRKENNGEINFAAD |
| CS1-R | 83 | MGDPKKKRKVIDIADLRTLGYSQQQQEKIKPKVRSTVAQH |
| | | HEALVGHGFTHAHIVALSQHPAALGTVAVKYQDMIAALPE |
| | | ATHEAIVGVGKQWSGARALEALLTVAGELRGPPLQLDTGQ |
| | | LLKIAKRGGVTAVEAVHAWRNALTGAPLNLTPQQVVAIAS |
| | | NNGGKQALETVQRLLPVLCQAHGLTPQQVVAIASNNGGKQ |
| | | ALETVQRLLPVLCQAHGLTPEQVVAIASNIGGKQALETVQ |
| | | ALLPVLCQAHGLTPEQVVAIASNIGGKQALETVQALLPVL |
| | | CQAHGLTPQQVVAIASNNGGKQALETVQRLLPVLCQAHGL |
| | | TPEQVVAIASNIGGKQALETVQALLPVLCQAHGLTPQQVV |
| | | AIASNNGGKQALETVQRLLPVLCQAHGLTPEQVVAIASNI |
| | | GGKQALETVQALLPVLCQAHGLTPEQVVAIASNIGGKQAL |
| | | ETVQALLPVLCQAHGLTPQQVVAIASNGGGKQALETVQRL |
| | | LPVLCQAHGLTPEQVVAIASHDGGKQALETVQRLLPVLCQ |
| | | AHGLTPEQVVAIASHDGGKQALETVQRLLPVLCQAHGLTP |
| | | QQVVAIASNNGGKQALETVQRLLPVLCQAHGLTPQQVVAI |
| | | ASNNGGKQALETVQRLLPVLCQAHGLTPEQVVAIASHDGG |
| | | KQALETVQRLLPVLCQAHGLTPQQVVAIASNGGGRPALES |
| | | IVAQLSRPDPSGSGSGGDPISRSQLVKSELEEKKSELRHK |
| | | LKYVPHEYIELIEIARNSTQDRILEMKVMEFFMKVYGYRG |
| | | KHLGGSRKPDGAIYTVGSPIDYGVIVDTKAYSGGYNLPIG |
| | | QADEMQRYVEENQTRNKHINPNEWWKVYPSSVTEFKFLFV |
| | | SGHFKGNYKAQLTRLNHITNCNGAVLSVEELLIGGEMIKA |
| | | GTLTLEEVRRKFNNGEINFAAD |

In some cases, the integration of the donor construct by homologous recombination at the targeted gene locus results in the reduction or suppression of the production of the targeted gene's product.

Therefore, in some cases, any of the donor constructs as described herewith can be integrated at a locus encoding a TCR component, HLA, B2M, PDCD1, CTLA4, TIM3, LAG3, CD69, IL2Ra, GM-CSF and/or CD52. As a consequence, in these cases, the targeted gene's expression is reduced or suppressed.

For instance, in some cases, a polynucleotide encoding the FAP-CAR as described herewith is integrated into the endogenous TRAC, B2M, or CD52 locus in the genome of said engineered immune cell, e.g. T-cell. In some cases, a polynucleotide encoding the tumor-CAR as described herewith is integrated into the endogenous PDCD1, CD25, GM-CSF, TIM3, or TIGIT locus in the genome of said engineered immune cell, e.g. T-cell.

In some cases, the vector can comprise an exogenous sequence coding for a FAP-CAR and/or a tumor-CAR, which is optionally co-expressed with a cytokine such as IL-12, IL-15, IL-7, or IL-2.

Gene targeted insertion of the sequences encoding CARs and/or other exogenous genetic sequences can be performed using AAV vectors, especially vectors from the AAV6 family or chimeric vectors AAV2/6 previously described by Sharma et al. (Brain Research Bulletin. (2010) 81 (2-3): 273-278).

One aspect thus relates to the transduction of such AAV vectors encoding a FAP-CAR or a tumor-CAR as described herewith, in human primary immune cells, such as primary T-cells, in conjunction with the expression of sequence-specific endonuclease reagents, such as TALE endonucleases, to increase gene integration at the loci previously cited.

Another aspect relates to the transduction of a recombinant lentiviral vector (rLV) encoding a CAR, such as a FAP-CAR or a tumor-CAR as described herewith, in human primary immune cells, in particular primary T-cells, that can be performed before or after introduction of a sequence-specific endonuclease reagent, such as a TALE endonuclease, to inactivate the genes previously cited (e.g. TRAC, TRBC, CD3, HLA, B2M, PDCD1, CTLA4, TIM3, LAG3, CD69, IL2Ra, GM-CSF and/or CD52).

In some cases, sequence-specific endonuclease reagents can be introduced into the cells by transfection, such as by electroporation of mRNA encoding said sequence-specific endonuclease reagents.

Accordingly, it is provided a method for inserting an exogenous nucleic acid sequence coding for a FAP-CAR or a tumor-CAR as described herein, at one of the previously cited locus in the cell's genome, which comprises at least one of the following steps:
transducing into said cell an AAV vector comprising an exogenous nucleic acid sequence encoding a FAP-CAR or a tumor-CAR and the sequences homologous to the targeted endogenous DNA sequence, and optionally:
inducing the expression of a sequence-specific endonuclease reagent to cleave said endogenous sequence at the locus of insertion.

The obtained insertion of the exogenous nucleic acid sequence may result into the introduction of genetic material and replacement of the endogenous sequence, and, thus, inactivation of the endogenous locus.

Another object relates to the AAV vector used in the method, which can comprise an exogenous coding sequence that is “promoterless,” the coding sequence being any of those referred to in this specification.

Many other vectors known in the art, such as plasmids, episomal vectors, linear DNA matrices, etc. can also be used to perform gene insertions at those loci by following present teachings.

As stated before, the DNA vector used for gene targeting integration as described herewith can comprise: (1) the exogenous nucleic acid to be inserted comprising the exogenous coding sequence of a FAP-CAR and/or the exogenous coding sequence of a tumor-CAR as described herewith, and (2) a sequence encoding the sequence-specific endonuclease reagent that promotes the insertion in the targeted locus. In some cases, said exogenous nucleic acid under (1) does not comprise a promoter sequence, whereas the sequence under (2) comprises its own promoter.

The DNA vector used for random integration as described herewith can comprise the exogenous nucleic acid to be inserted comprising (i) a constitutive promoter and, operably linked to said promoter, the exogenous coding sequence of a FAP-CAR, as described herewith, and/or (ii) an inducible promoter and, operably linked to said promoter, the exogenous coding sequence of a tumor-CAR, as described herewith.

It is to be understood that, as it derives from the meaning of the term “exogenous sequence” provided herewith, the sequences comprised in the DNA vector to be integrated are necessarily “exogenous” since it is intended that they are added to the cell's genome. Thus, in this situation the adjective “exogenous” could have been omitted.

According to another aspect, when said CAR is a multi-chain CAR, the nucleic acid under (1) further comprises an Internal Ribosome Entry Site (IRES) or “self-cleaving” 2A peptides, such as T2A, P2A, E2A or F2A, so that the exogenous coding sequence inserted is multi-cistronic. The IRES of 2A Peptide can precede or follow said exogenous coding sequence.

The exogenous polynucleotide sequences encoding said FAP-CAR and/or tumor-CAR can also be introduced into the immune cells, e.g. T-cells or NK-cells, or into iPSCs, by using a viral vector, such as lentiviral vectors. The present disclosure thus provides with viral vectors encoding FAP-CARs and/or tumor-CARs as described herein.

In some cases, lentiviral or AAV vectors as contemplated herewith can comprise sequences encoding a CAR separated by a T2A or P2A sequence, as forming one transcriptional unit. In lentiviral vectors said sequences coding for the FAP-CAR as described herewith can form an expression cassette transcribed under control of a constitutive exogenous promoter, such as a EF1alpha promoter derived from the human EF1A1 gene. In some cases, in lentiviral vectors, said sequences coding for the tumor-CAR as described herewith can form an expression cassette transcribed under control of an inducible exogenous promoter, such as a polynucleotide comprising at least one NFAT responsive element comprising, for instance, SEQ ID NO: 114 or SEQ ID NO: 115.

In some cases, the engineered cells are made by a process comprising random integration, in the genome of said cells, of a lentiviral vector comprising a constitutive promoter sequence and a polynucleotide encoding a FAP-CAR as described herewith.

In some cases, the engineered cells are made by a process comprising random integration, in the genome of said cells, of a lentiviral vector comprising an inducible promoter sequence and a polynucleotide encoding a tumor-CAR as described herewith.

In some cases, the engineered cells are made by a process comprising random integration, in the genome of said cells, of one lentiviral vector comprising a cassette comprising a constitutive promoter and a polynucleotide encoding a FAP-CAR as described herewith and a cassette comprising an inducible promoter and a polynucleotide encoding a tumor-CAR as described herewith.

In some cases, the engineered cells are made by a process comprising targeted integration of the exogenous sequences encoding the FAP-CAR and/or tumor-CAR as described herewith.

Thus, in some cases, the engineered cells are made by a process comprising targeted integration, in the genome of said cells, of a polynucleotide encoding a FAP-CAR as described herewith through sequence-specific endonuclease-mediated cDNA insertion at a constitutively expressed gene locus in the cells' genome. In these cases, said cDNA comprises a polynucleotide encoding a FAP-CAR as described herewith and the constitutively expressed gene locus is a locus controlled by an endogenous constitutive promoter as defined herewith. In these cases, the expression of said FAP-CAR is controlled by said endogenous constitutive promoter.

In some cases, the engineered cells are made by a process comprising targeted integration, in the genome of said cells, of a polynucleotide comprising an exogenous constitutive promoter and, operably linked to said promoter, an exogenous nucleic acid sequence encoding a FAP-CAR as described herewith, through sequence-specific endonuclease-mediated insertion at a constitutively expressed gene locus in the cells' genome. In these cases, expression of said FAP-CAR is controlled by said exogenous constitutive promoter.

In some cases, the engineered cells are made by a process comprising targeted integration, in the genome of said cells, of a polynucleotide encoding a tumor-CAR as described herewith through sequence-specific endonuclease-mediated cDNA insertion at an inducible gene locus in the cells' genome. In these cases, said cDNA comprises a polynucleotide encoding a tumor-CAR as described herewith and the inducible gene locus is controlled by an inducible promoter as defined herewith. In these cases, the expression of said tumor-CAR is controlled by said endogenous inducible promoter.

In some cases, the engineered cells are made by a process comprising targeted integration, in the genome of said cells, of a polynucleotide comprising an exogenous inducible promoter and, operably linked to said promoter, an exogenous nucleic acid sequence encoding a tumor-CAR as described herewith, through sequence-specific endonuclease-mediated insertion at an inducible gene locus in the cells' genome. In these cases, expression of said tumor-CAR is controlled by said exogenous inducible promoter.

Another aspect relates to a set of vectors or a kit for producing the engineered immune cells as described herewith.

In one aspect is provided a set of vectors comprising at least one vector comprising a nucleic acid sequence encoding a FAP-CAR placed under the transcriptional control of a constitutive promoter and at least one vector comprising a nucleic acid sequence encoding a tumor-CAR placed under the transcriptional control of an inducible promoter.

In another aspect is provided a kit comprising:
(a) at least one vector comprising a nucleic acid sequence encoding a FAP-CAR as described herewith placed between a Left-Homology Region and a Right-Homology Region, wherein said Regions are homologous to the locus targeted by the endonuclease of (b); and
(b) at least one sequence-specific endonuclease targeting one constitutively expressed gene locus such as EF1A, CD52, GAPDH, hPGK, UBC, TRAC, TRBC, TRGC, TRDC, B2M, CD5, CS1, CD45, RPBSA, CD4, or CD8, and/or
(c) at least one vector comprising a nucleic acid sequence encoding a tumor-CAR as described herewith placed between a Left-Homologous Region and a Right-Homology Region, wherein said Regions are homologous to the locus targeted by the endonuclease of (d), and
(d) at least one sequence-specific endonuclease targeting one inducible gene locus such as PDCD1, CD25, TIM3, TIGIT, CCL1, NR4A3, EGR3, GOS2, IL22, RGS16, FASLG, RDH10, CSF1, GM-CSF, LAG3, CTLA-4, IL10, NUR77, or FOXP3.

In another aspect is provided a kit comprising:
(a) at least one vector comprising a nucleic acid sequence comprising a constitutive promoter and, operably linked to said promoter, a nucleic acid sequence encoding a FAP-CAR as described herewith; and
(b) at least one vector comprising a nucleic acid sequence comprising an inducible promoter and, operably linked to said promoter, a nucleic acid sequence encoding a tumor-CAR as described herewith.

In another aspect is provided a kit comprising:
(a) at least one vector comprising a nucleic acid sequence comprising a constitutive promoter and, operably linked to said promoter, a nucleic acid sequence encoding a FAP-CAR as described herewith; and
(b) at least one vector comprising a nucleic acid sequence encoding a tumor-CAR as described herewith placed between a Left- and a Right-Homology Regions, wherein said Regions are homologous to the locus targeted by the endonuclease of (c), and
(c) at least one sequence-specific endonuclease targeting one inducible gene locus such as PDCD1, CD25, TIM3, TIGIT, CCL1, NR4A3, EGR3, GOS2, IL22, RGS16, FASLG, RDH10, CSF1, GM-CSF, LAG3, CTLA-4, IL10, NUR77, FOXP3.

In a still further aspect is provided a kit comprising:
(a) at least one vector comprising a nucleic acid sequence encoding a FAP-CAR as described herewith placed between a Left- and a Right-Homology Regions, wherein said Regions are homologous to the locus targeted by the endonuclease of (b); and
(b) at least one sequence-specific endonuclease targeting one constitutively expressed gene locus such as EF1A, CD52, GAPDH, hPGK, UBC, TRAC, TRBC, TRGC, TRDC, B2M, CD5, CS1, CD45, RPBSA, CD4, or CD8, and/or
(c) at least one vector comprising a nucleic acid sequence comprising an inducible promoter and, operably linked to said promoter, a nucleic acid sequence encoding a tumor-CAR as described herewith.

In another aspect is provided a kit comprising:
(a) at least one vector comprising a cassette comprising a constitutive promoter and, operably linked to said promoter, a nucleic acid sequence encoding a FAP-CAR as described herewith; wherein said cassette of (a) is placed between a Left- and a Right-Homology Regions, wherein said Regions are homologous to the locus targeted by the endonuclease of (b); and
(b) at least one sequence-specific endonuclease targeting a targeted gene locus, and/or
(c) at least one vector comprising a cassette comprising an inducible promoter and, operably linked to said promoter, a nucleic acid sequence encoding a tumor-CAR as described herewith; wherein said cassette of (c) is placed between a Left- and a Right-Homology Regions, wherein said Regions are homologous to the locus targeted by the endonuclease of (d), and
(d) at least one sequence-specific endonuclease targeting a targeted gene locus.

In this later aspect, as the FAP-CAR and tumor-CAR are under the control of the specified exogenous promoters, there is no restriction as to whether said targeted gene locus of (b) and (d) need to be inducible or constitutively expressed. Thus, in this later aspect said targeted gene locus of (b) can be inducible or constitutively expressed, and said targeted gene locus of (d) can be inducible or constitutively expressed.

In some of the kits, the sequence-specific endonuclease of (b) is a TALE nuclease. In some cases, said TALE nuclease of (b) targets one endogenous constitutively expressed gene locus selected from the group consisting of EF1A, CD52, GAPDH, hPGK, UBC, TRAC, TRBC, TRGC, TRDC, B2M, CD5, CS1, CD45, RPBSA, CD4, and CD8. In some cases, said TALE nuclease of (b) targets one endogenous constitutively expressed gene locus selected from the group consisting of EF1A, TRAC, B2M, CD52, CS1, CD45, CD5, and GAPDH. In some cases, said TALE nuclease of (b) targets one endogenous constitutively expressed gene locus selected from the group consisting of EF1A, TRAC, B2M, and CD52.

In some of the kits, the sequence-specific endonuclease of (d) or (c) is a TALE nuclease.

In some cases, said TALE nuclease of (d) or (c) targets one inducible gene locus selected from the group consisting of PDCD1, CD25, TIM3, TIGIT, CCL1, NR4A3, EGR3, GOS2, IL22, RGS16, FASLG, RDH10, CSF1, GM-CSF, LAG3, CTLA-4, IL10, NUR77, and FOXP3.

In some cases, said TALE nuclease of (d) or (c) targets one inducible gene locus selected from the group consisting of PDCD1, CD25, GM-CSF, TIM3, and TIGIT.

In some cases, said TALE nuclease of (d) or (c) can target one inducible gene locus that is PDCD1.

In some instances, when a vector of said kits does not comprise a promoter controlling the transcription of the exogenous nucleic acid sequence comprised in the vector, the transcription of said exogenous nucleic acid sequence will be controlled by the endogenous promoter of the targeted endogenous locus, after integration in the cell's genome.

In some instances, when a vector of said kits comprises a promoter controlling the transcription of the exogenous nucleic acid sequence comprised in the vector, the transcription of said exogenous nucleic acid sequence will be controlled by the exogenous promoter, after integration in the cell's genome.

### Activation and Expansion of Immune Cells

Whether prior to or after genetic modification, the immune cells described herewith can be activated or expanded, even if they can activate or proliferate independently of antigen binding mechanisms. T-cells, for example, can be activated and expanded using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and 7,572,631. T-cells can be expanded in vitro or in vivo. T-cells are generally expanded by contact with an agent that stimulates a CD3 TCR complex and a co-stimulatory molecule on the surface of the T-cells to create an activation signal for the T-cell. For example, chemicals such as calcium ionophore A23187, phorbol 12-myristate 13-acetate (PMA), or mitogenic lectins like phytohemagglutinin (PHA) can be used to create an activation signal for the T-cell.

As non-limiting examples, T-cell populations may be stimulated in vitro such as by contact with an anti-CD3 antibody, or antigen-binding fragment thereof, or an anti-CD2 antibody immobilized on a surface, or by contact with a protein kinase C activator (e.g. bryostatin) in conjunction with a calcium ionophore. For co-stimulation of an accessory molecule on the surface of the T-cells, a ligand that binds the accessory molecule is used. For example, a population of T-cells can be contacted with an anti-CD3 antibody and an anti-CD28 antibody, under conditions appropriate for stimulating proliferation of the T-cells. Conditions appropriate for T-cell culture include an appropriate media (e.g. Minimal Essential Media or RPMI Media 1640 or, X-vivo 5, (Lonza)) that may contain factors necessary for proliferation and viability, including serum (e.g. fetal bovine or human serum), interleukin-2 (IL-2), insulin, IFN-g, IL-4, IL-7, GM-CSF, IL-10, IL-12, IL-15, TGFp, and TNF- or any other additives for the growth of cells known to the skilled artisan. Other additives for the growth of cells include, but are not limited to, surfactant, plasmanate, and reducing agents such as N-acetyl-cysteine and 2-mercaptoethanoi. Media can include RPMI 1640, A1M-V, DMEM, MEM, a-MEM, F-12, X-Vivo 1, and X-Vivo 20, OptTmizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T-cells. Antibiotics, e.g. penicillin and streptomycin, are included only in experimental cultures, not in cultures of cells that are to be infused into a subject. The target cells are maintained under conditions necessary to support growth, for example, an appropriate temperature (e.g. 37° C.) and atmosphere (e.g. air plus 5% CO₂). T-cells that have been exposed to varied stimulation times may exhibit different characteristics.

In some cases, said cells can be expanded by co-culturing with tissue or cells. Said cells can also be expanded in vivo, for example in the subject's blood after administrating said cell into the subject.

Any biological activity exhibited by the engineered immune cell expressing a CAR can be determined, including, for instance, cytokine production and secretion, degranulation, proliferation, or any combination thereof.

In some instances, the biological activity determined in step (iii) is cytokine secretion, cell proliferation, or both.

The biological activities can be measured by methods well known by the skilled person, such as by in vitro and/or ex vivo methods.

Secretion of any cytokine can be measured, e.g. secretion of IFNγ, TNFα, can be determined. Standard methods to determine cytokine secretion includes ELISA, flow cytometry. These methods are described for instance in Sachdeva et al. (Front Biosci, 2007, 12:4682-95) and Pike et al. (2016) (Methods in Molecular Biology, vol 1458. Humana Press, New York, NY).

The level of cytokine secretion can be measured, for instance, as the maximum level of cytokine (e.g. IFNγ) secreted per CAR-expressing immune cell (e.g. CAR-T cell), e.g. maximum amount of IFNγ secreted per CAR-T cell.

To evaluate “degranulation,” standard methods can be used, including for instance CD107a degranulation assay or measurement of secreted Granzyme B or Perforin (such as described in Lorenzo-Herrero et al, (Methods Mol Biol (2019) 1884:119-130), Betts et al. Methods in Cell Biology (2004) 75:497-512).

To evaluate “proliferation” activity, standard methods can be carried out, which are mainly based on methods involving measurement of DNA synthesis, detection of proliferation-specific markers, measurement of successive cell divisions by the use of cell membrane binding dyes, measurement of cellular DNA content and measurement of cellular metabolism.

In some cases, the methods described herewith allow producing engineered T-cells within a limited time frame of about 15 to 30 days, for instance between 15 and 20 days or between 18 and 20 days so that the cells keep their full immune therapeutic potential, especially with respect to their cytotoxic activity.

These cells can be from or be members of populations of cells, which can originate from a single donor or patient. In some cases, these populations of cells can be expanded under closed culture recipients to comply with highest manufacturing practices requirements and can be frozen prior to infusion into a patient, thereby providing “off the shelf” or “ready to use” therapeutic compositions.

In some cases, a significant number of cells originating from the same leukapheresis can be obtained, which can be important to obtain sufficient doses for treating a patient. Although variations between populations of cells originating from various donors may be observed, the number of immune cells procured by a leukapheresis is generally about from 10⁸ to 10¹⁰ cells of PBMC. PBMC comprises several types of cells: granulocytes, monocytes and lymphocytes, among which from 30 to 60% of T-cells, which generally represents between 10⁸ to 10⁹ of primary T-cells from one donor.

In some cases, methods described herewith generally end up with a population of engineered cells that reaches generally more than about 10⁸ T-cells, more generally more than about 10⁹ T-cells, even more generally more than about 10¹⁰ T-cells, and usually more than 10¹¹ T-cells. In some cases, the T-cells are gene edited in at least at two different loci.

Such compositions or populations of engineered cells can therefore be used as a therapeutic; especially for treating any of the cancers herein, for example for the treatment of solid tumors in patients such as melanomas, neuroblastomas, gliomas or carcinomas such as lung, breast, colon, prostate or ovary tumors in a patient in need thereof.

Also encompassed herewith is a therapeutically effective population of immune cells comprising at least 30%, at least 50%, or at least 80% of engineered cells as described herewith.

The present document discloses, for instance, populations of primary TCR negative immune cells, such as T-cells, originating from a single donor, wherein at least 20%, at least 30%, at least 50%, at least 90%, at least 95%, at least 96%, or at least 97% of the cells in said population have been genetically modified using sequence-specific reagents to become TCR negative.

By “TCR negative immune cell” is meant an immune cell, such as a T-cell or NK-cell, in which expression of TCR is not detectable by standard methods based on antibodies such as Flow-cytometry, Western-blot, ELISA. TCR negative immune cells include immune cells which have two of the endogenous alleles encoding a component of the T-cell receptor that have been genetically modified (e.g. disrupted), so that TCR presence at the cell surface of said engineered cells is suppressed and/or not detectable. TCR negative immune cells also include immune cells which, in their natural non-engineered state, generally do not express TCR gene, such as is the case of NK cells.

By “CD52 negative immune cell” is meant an immune cell, such as a T-cell or NK-cell, in which expression of CD52 is not detectable by standard methods based on antibodies such as Flow-cytometry, Western-blot, ELISA. CD52 negative immune cells include immune cells which have two of the endogenous alleles encoding CD52 that have been genetically modified (e.g. disrupted), so that CD52 presence at the cell surface of said engineered cells is suppressed and/or not detectable.

By “B2M negative immune cell” is meant an immune cell, such as a T-cell or NK-cell, in which expression of 32M is not detectable by standard methods based on antibodies such as Flow-cytometry, Western-blot, ELISA. B2M negative immune cells include immune cells which have two of the endogenous alleles encoding 2M that have been genetically modified (e.g. disrupted), so that B2M presence at the cell surface of said engineered cells is suppressed and/or not detectable.

By “PDCD1 negative immune cell” is meant an immune cell, such as a T-cell or NK-cell, in which expression of PD1 is not detectable by standard methods based on antibodies such as Flow-cytometry, Western-blot, ELISA. PDCD1 negative immune cells include immune cells which have two of the endogenous alleles encoding PD1 that have been genetically modified (e.g. disrupted), so that PD1 presence at the cell surface of said engineered cells is suppressed and/or not detectable.

By “GM-CSF negative immune cell” is meant an immune cell, such as a T-cell or NK-cell, in which expression of GM-CSF is not detectable by standard methods based on antibodies such as Flow-cytometry, Western-blot, ELISA. GM-CSF negative immune cells include immune cells which have two of the endogenous alleles encoding GM-CSF that have been genetically modified (e.g. disrupted), so that GM-CSF presence at the cell surface of said engineered cells is suppressed and/or not detectable.

### Methods of Treatment and Products for Use in Immunotherapy

An aspect relates to a pharmaceutical composition comprising a therapeutically effective amount of immune cells as described herewith.

Also described herewith is a composition comprising a therapeutically effective amount of immune cells as described herewith, for use in the treatment of a cancer, such as a cancer characterized by the presence of FAP in the tumor microenvironment.

Also contemplated herewith is a method of treatment of a cancer, such as a cancer characterized by the presence of FAP in the tumor microenvironment, comprising administering a therapeutically effective amount of engineered immune cells as described herewith.

The cancer that can be treated with the compositions, cells, or method of treatment described herewith are not limiting.

Said cancer can be a solid tumor or an haematological cancer.

Said cancer expressing a solid tumor antigen can be selected from any one of breast cancer, ovarian cancer, endometrial cancer, cervical cancer, bladder cancer, renal cancer, melanoma, lung cancer, prostate cancer, testicular cancer, thyroid cancer, brain cancer, esophageal cancer, gastric cancer, pancreatic cancer, colorectal cancer, or liver cancer.

Examples of said cancer expressing a solid tumor antigen include breast cancer (e.g. triple-negative breast cancer), pancreatic cancer, and lung cancer (e.g. malignant pleural mesothelioma).

Said haematological cancer characterized by the presence of FAP in the tumor microenvironment can be selected from the group consisting of myelofibrosis, myelodysplastic syndromes, acute myeloid leukemia, non-Hodgkin's lymphoma, multiple myeloma.

All of the above listed cancers can be treated with the engineered immune cells or the pharmaceutical composition described herein.

The cancers advantageously treated with the engineered immune cells or pharmaceutical composition described herein are those for which the tumor antigen to be targeted is also present in normal healthy tissues.

In some cases, the cancer is an ovarian cancer and the tumor antigen is selected from one or more of mesothelin, glycoprotein 72 (TAG72), MUC16, Her2, 5T4, and FRα.

In some cases, the cancer is a breast cancer and the tumor antigen is selected from one or more of MUC28z, NKG2D, HRG1β, and HER2.

In some cases, the cancer is a prostate cancer and the tumor antigen is selected from one or more of prostate stem cell antigen (PSCA) and prostate-specific membrane antigen (PSMA).

In some cases, the cancer is a renal cancer and the tumor antigen is carboxy-anhydrase-IX (CA-IX).

In some cases, the cancer is a gastric cancer and the tumor antigen is selected from one or more of Trop2, claudin18.2, NKG2D, folate receptor 1 (FOLR1), and HER2.

In some cases, the cancer is a pancreatic cancer and the tumor antigen is selected from one or more of mesothelin, MUC1, CXCR2, B7-H3, CD133, CD24, PSCA, CEA, and Her-2.

In some cases, the cancer is a lung cancer and the tumor antigen is selected from one or more of mesothelin, receptor tyrosine kinase-like orphan receptor 1-specific (ROR1), EGFRvIII, erythropoietin-producing hepatocellular carcinoma A2 (EphA2), PSCA, MUC1, and DLL3.

In some cases, the cancer is a liver cancer and the tumor antigen is selected from one or more of MUC1, CEA, glypican-3, and epithelial cell adhesion molecule (EPCAM).

In some cases, the cancer is a colorectal cancer and the tumor antigen is selected from one or more of MUC1, NKG2D, CD133, GUCY2C (Guanylate Cyclase 2C), TAG-72 Doublecortin-like kinase 1 (DCLK1), and CEA.

In some cases, the haematological cancer is myelofibrosis and the tumor antigen is CALR.

In some cases, the haematological cancer is myelodysplastic syndromes and the tumor antigen is selected from one or more of CD123, CD33, and NKG2D.

In some cases, the haematological cancer is acute myeloid leukemia and the tumor antigen is selected from one or more of CD123, CLL-1, IL1RAP, CD33, CD135, CD70, CD44, CD276, ILT3, CD7, CD47, TIM3, CD96, and VISTA.

In some cases, the haematological cancer is acute lymphocytic leukemia and the tumor antigen is selected from one or more of CD19, CD22, CD79a, CD10, CD2, CD3, CD4, CD5, CD7, CD8, CRLF2, and CD38.

In some cases, the haematological cancer is non-Hodgkin's lymphoma and the tumor antigen is selected from one or more of CD19, CD20, CD22, CD80, CD37, CD79, CD30, CD70, and CD38.

In some cases, the haematological cancer is multiple myeloma and the tumor antigen is selected from one or more of BCMA, CD19, CD138, CS1, CD38, TACI, APRIL, GPRC5D, and CD44v6.

The treatments involving the engineered primary immune cells described herewith can be ameliorating, curative or prophylactic.

In some cases, the patient can undergo preparative lymphodepletion—the temporary ablation of the immune system—prior to administration of the engineered T-cells. In some cases, the lymphodepletion is only partial and not a complete ablation of the patient's immune system. In some cases, a combination of IL-2 treatment and preparative lymphodepletion can enhance persistence of a cellular therapeutic.

In some cases, the engineered immune cells, such as T-cells, described herewith can be administered in an amount of about 10⁶ to 10⁹ cells/kg, with or without a course of lymphodepletion, for example by administering cyclophosphamide and/or fludarabine, and/or alemtuzumab.

In some cases, the cells or population of cells comprising the engineered immune cells, such as T-cells, described herewith are administered in an amount of about 10⁴-10⁹ cells per kg body weight, from about 10⁵ to 5×10⁶ cells/kg body weight, or from about 10⁵ to 10⁶ cells/kg body weight, including all integer values of cell numbers within those ranges. Dosing in CAR-T cell therapies may for example involve administration of from 10⁵ or 10⁶ to 10⁹ cells/kg, with or without a course of lymphodepletion, for example with fludarabine, cyclophosphamide or alemtuzumab, or any combination thereof.

The cells or population of cells can be administered in one or more doses. In some cases, the effective amount of cells are administered as a single dose. In some cases, the effective amount of cells are administered as more than one dose over a period of time. Timing of administration is within the judgment of managing physician and depends on the clinical condition of the patient. The cells or population of cells may be obtained from any source, such as a blood bank or a donor. While individual needs vary, determination of optimal ranges of effective amounts of a given cell type for a particular disease or conditions are within the skill of one in the art.

An effective amount of engineered immune cells, such as CAR-T cells, means an amount which provides a therapeutic or prophylactic benefit. The dosage administered will be dependent upon the age, health and weight of the recipient, kind of concurrent treatment, if any, frequency of treatment and the nature of the effect desired.

The treatment with the engineered immune cells described herewith may be carried out in further combination with one or more therapies against cancer selected from the group of antibodies therapy, chemotherapy, cytokines therapy, dendritic cell therapy, gene therapy, hormone therapy, laser light therapy and radiation therapy.

For example, the treatment with the engineered immune cells as described herewith can be carried out in combination with the administration of an immune checkpoint antagonist, that can be administered intravenously in an amount of about 200 mg to 400 mg including all integer values within those ranges.

What is described herewith with engineered T-cells comprising an inactivated TCR and expressing a FAP-CAR constitutively and a tumor-CAR upon activation of the T-cells can equally be applied to engineered Natural Killer cells expressing a FAP-CAR constitutively and a tumor-CAR upon activation of the NK cells.

Such engineered NK cells are naturally TCR negative. The NK cells described herewith can originate from a donor or from a cell line such as NK92 cell line. In some cases, the engineered NK cells derive from engineered iPSCs as described herewith which have been differentiated into NK cells.

Optionally, said engineered NK cells have a reduced expression of B2M gene mediated by gene inactivation and/or by gene silencing and/or by inserting into the B2M locus of said NK-cells' genome at least one exogenous polynucleotide encoding a CAR as defined herewith.

Said engineered NK cells may have a reduced expression of CD52 gene mediated by gene inactivation and/or by gene silencing and/or by inserting into the CD52 locus of said NK-cells' genome at least one exogenous polynucleotide encoding a CAR as defined herewith.

In some cases, said engineered NK cells comprise either the CD52 or the B2M gene inactivated.

Thus, is also provided herewith an engineered NK-cell comprising:
a) an exogenous nucleic acid sequence encoding a chimeric antigen receptor (CAR) targeting the Fibroblast Activation Protein (FAP) (“FAP-CAR”) placed under the transcriptional control of an exogenous or endogenous constitutive promoter; and
b) an exogenous nucleic acid sequence encoding a chimeric antigen receptor (CAR) targeting a tumor antigen (“tumor-CAR”) placed under the transcriptional control of an exogenous or endogenous inducible promoter;
wherein said exogenous nucleic acid sequences a) and b) are integrated in the cell's genome, and
wherein the expression of the tumor-CAR is inducible upon activation of the NK cell; and
wherein, optionally, the NK-cell has been genetically modified to suppress or repress expression of at least one gene controlling MHC complex surface presentation, such as B2M or CIITA, in the NK-cell.

Similar FAP-CARs and tumor-CARs as described herewith can be expressed in said NK cells to produce engineered FAP/tumor-CAR-NK, which can be used in methods of treatment of a cancer characterized by the presence of FAP in the tumor microenvironment, such as solid tumor and haematological cancers, as described herewith.

Thus, are also described herewith a pharmaceutical composition comprising engineered NK-cells comprising (i) an exogenous nucleic acid sequence encoding a FAP-CAR placed under the transcriptional control of an exogenous or endogenous constitutive promoter, (ii) an exogenous nucleic acid sequence encoding a tumor-CAR placed under the transcriptional control of an exogenous or endogenous inducible promoter, and (iii) optionally comprising an inactivated β2M gene.

A still other aspect described herewith is a pharmaceutical composition as described above for use in the treatment of a cancer characterized by the presence of FAP in the tumor microenvironment, such as solid tumors and haematological cancers; wherein said exogenous nucleic acid sequences a) and b) are integrated in the cell's genome, and wherein the expression of the tumor-CAR is inducible upon activation of the NK cell.

The above written description provides a manner and process of making and using the invention such that any person skilled in the art is enabled to make and use the same, this enablement being provided in particular for the subject matter of the appended claims, which make up a part of the original description.

Where a numerical limit or range is stated herein, the endpoints are included. Also, all values and subranges within a numerical limit or range are specifically included as if explicitly written out.

Having generally described this invention, a further understanding can be obtained by reference to certain specific examples, which are provided herein for purposes of illustration only, and are not intended to limit the scope of the claimed invention.

### EXAMPLES

### Example 1: Materials

### Tale-Nucleases Targeting TRAC, PDCD1, or CS1

TALEN-mRNAs targeting TRAC (SEQ ID NO: 74 and SEQ ID NO: 75), PDCD1 (SEQ ID NO: 72 and SEQ ID NO: 73) or CS1 (SEQ ID NO: 82 and SEQ ID NO: 83) were produced by Trilink.

### AAV Constructs

FAP-CAR construct was inserted in frame with TRAC locus and peptide 2A in an AAV vector. The TRAC-FAP-CAR donor matrix is composed of 300 bp of the TRAC left and right Homology arms, a self-cleaving 2A peptide allowing the expression of the FAP-CAR (SEQ ID NO: 111).

Mesothelin-CAR (“Meso-CAR”) construct was inserted in frame with PDCD1 locus with a peptide 2A in an AAV vector. The PDCD1-Meso-CAR donor matrix is composed of 300 bp of the PDCD1-left and right Homology arms, a self-cleaving 2A peptide allowing the expression of the Meso-CAR. Additionally, the Meso-CAR is followed by an EF1A promoter driving PDCD1 independent expression of a truncated surface protein DLNGFR. DLNGFR expression is thus used as a reporter for matrix insertion at PDCD1 locus as well as a method of cell enrichment as described below (SEQ ID NO: 112).

AAV particles comprising either TRAC-FAP-CAR or PDCD1-Meso-CAR vectors were produced by Vigene.

### rLV Construct

FAP-CAR expression cassette was inserted randomly in the genome using recombinant lentiviral vector comprising the FAP-CAR coding sequence under the EF1A promoter (SEQ ID NO: 113). The lentivirus particles were produced by Flash Therapeutics.

### Example 2: Generation of CAR-T Cells Having a Constitutive Expression of a FAP-CAR and an Inducible Expression of a Meso-CAR, by AAV Transduction

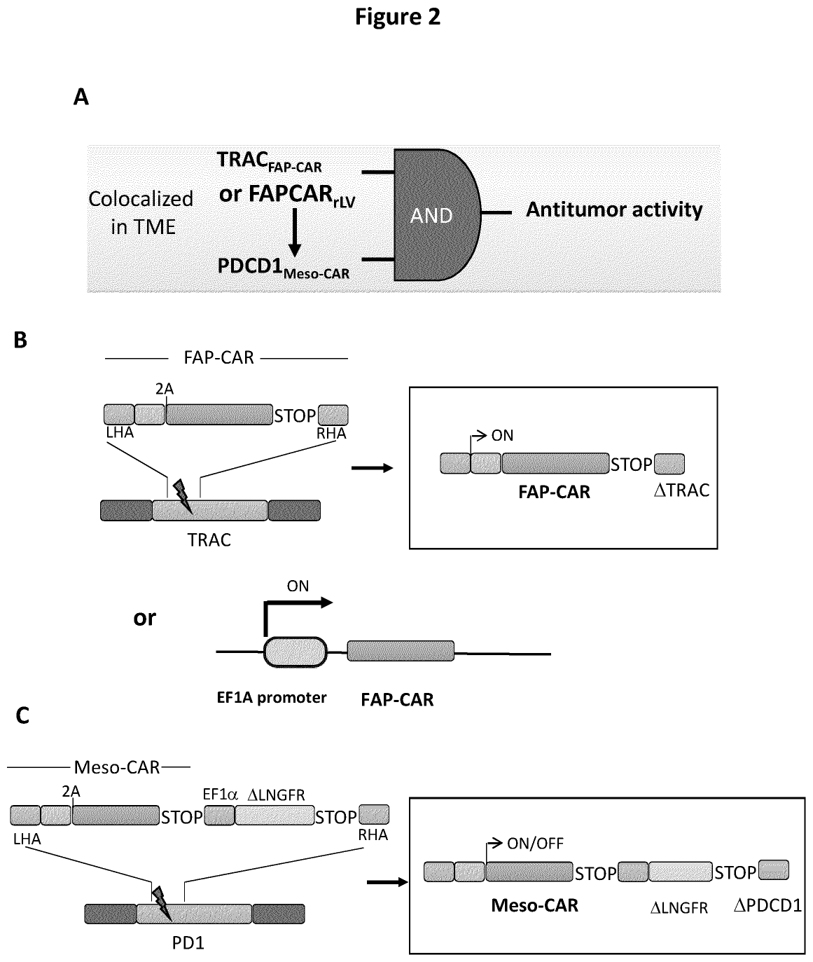

This Example describes the generation of universal CAR-T cells having a constitutive expression of a FAP-CAR and an inducible expression of a Meso-CAR. The FAP-CAR construct was inserted at the endogenous TRAC locus, whereas the Meso-CAR construct was inserted at the endogenous PDCD1 locus. The expression of FAP-CAR and Meso-CAR was driven by the endogenous TRA promoter and the PDCD1 promoter, respectively (FIG. 2A, B and C).

### Engineered CAR-T Cells

To express a FAP-CAR constitutively on the surface of primary T cells, cryopreserved PBMC were thawed at 37° C., washed and re-suspended in OpTmizer medium supplemented with AB human serum (5%) for an overnight incubation at 37° C. in 5% CO₂ incubator. Cells were then activated with Transact in OpTmizer medium supplemented with AB human serum (5%) and recombinant human interleukin-2 (rhIL-2, 350 IU/ml) in a CO₂ incubator (culture medium). Three days after activation, T-cells were electroporated with 5 μg of each mRNA encoding a TALEN® arm specific for TRAC (SEQ ID NO: 74 and SEQ ID NO: 75) and for PDCD1 (SEQ ID NO: 72 and SEQ ID NO: 73). Transfection was performed using Pulse Agile technology by applying two 0.1 mS pulses at 800 V followed by four 0.2 mS pulses at 130 V in 0.4 cm gap cuvettes in Cytoporation buffer T (BTX Harvard Apparatus, Holliston, Massachusetts). The electroporated cells were then immediately transferred into prewarmed OpTmizer serum-free media and incubated at 37° C. for 15 min. The cells were then concentrated and incubated in the presence of TRAC-FAP-CAR AAV particles (MOI=1.1E5 vg/cells) and PDCD1-Meso-CAR AAV particles (MOI=7.5E4 vg/cells), comprising the donor matrices depicted in FIGS. 2B and 2C respectively. After 2 h of culture at 30° C., OpTmizer media supplemented by 10% AB serum and IL-2 was added to the cell suspension, and the mix was incubated for 16 h under the same culture conditions. Cells were subsequently cultivated at 37° C. in the presence of 5% CO₂.

### Enrichment of PDCD1-Meso-CAR-Ts by Magnetic Sorting of DLNGFR Positive Cells

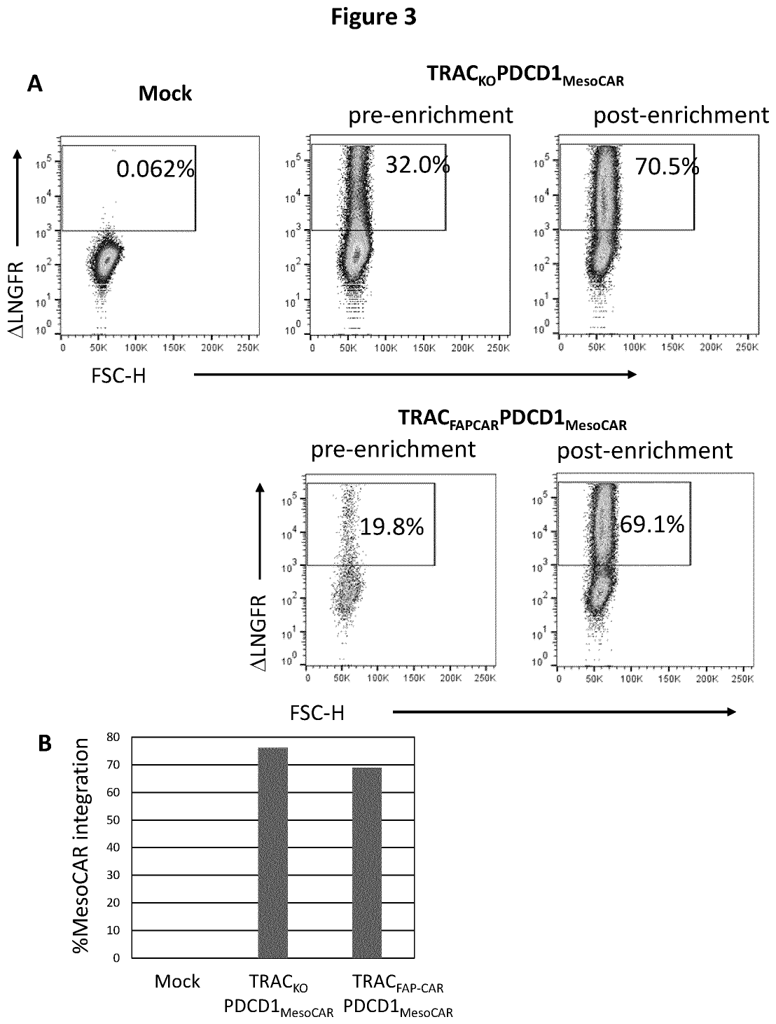

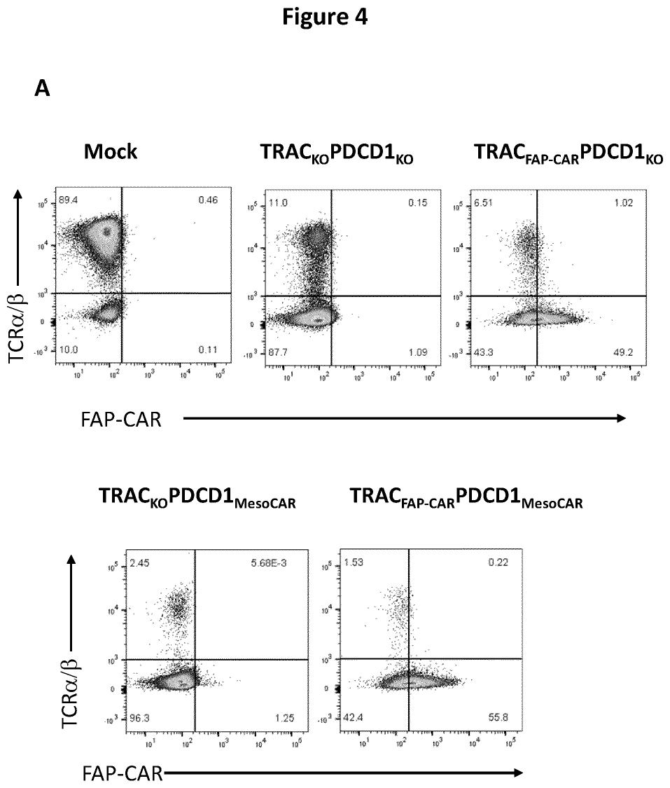

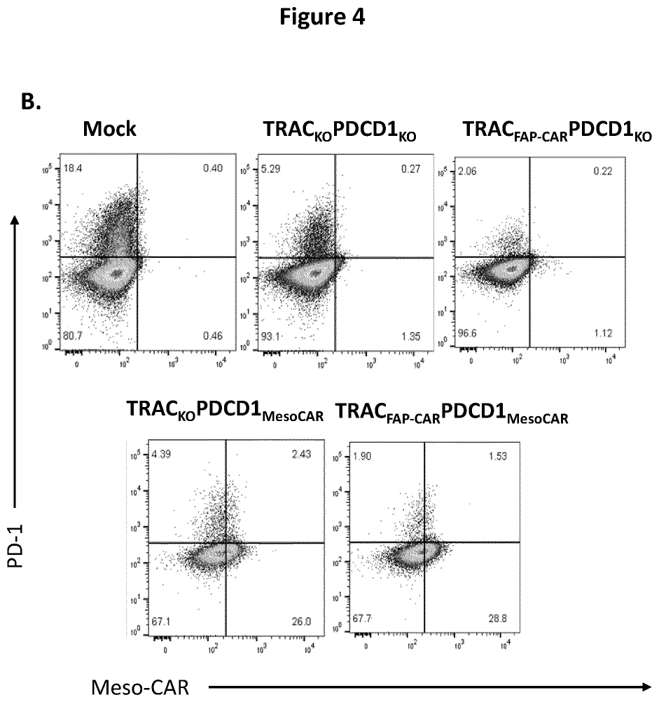

As described above, cells with integration of the Meso-CAR matrix at PDCD1 locus express a non-functional truncated LNGFR (DLNGFR) protein on their surface. DLNGFR positive T cells were positively selected by magnetic sorting using MACSelect™ LNGFR MicroBeads, as per manufacturer protocol (Miltenyi). Positive selection of DLNGFR+ cells (FIG. 3A) led to significant enrichment of PDCD1 integrated Meso-CAR-T cells, as measured by ddPCR (FIG. 3B). Subsequently, these cells were analysed for TRAC knockout, FAP-CAR expression (FIG. 4A) and Meso-CAR expression post activation with PMA (20 μM)/Ionomycin (800 ng/ml) for 24 h by flow cytometry (FIG. 4B). More than 90% of TRAC knockout could be achieved and more than 50% of the edited T-cells expressed FAP-CAR (FIG. 4A). In addition, PDCD1 positive cells were reduced from 18% (in Mock control) down to 2 to 5% (FIG. 4B).

### Example 3: Generation of CAR-T Cells Having a Constitutive Expression of a FAP-CAR by Lentiviral Transduction and an Inducible Expression of a Meso-CAR by AAV-Mediated Targeted Integration

T cells were electroporated with TALEN to knockout TRAC and PDCD1 genes, transduced with lentivirus to constitutively express CAR against FAP protein and with the AAV for targeted integration of Meso-CAR at PDCD1 locus.

To express a FAP-CAR on the surface of primary T cells, cryopreserved PBMC were thawed at 37° C., washed and re-suspended in OpTmizer medium supplemented with AB human serum (5%) for overnight incubation at 37° C. in 5% CO₂ incubator. Cells were then activated with Transact in OpTmizer medium supplemented with AB human serum (5%) and recombinant human interleukin-2 (rhIL-2, 350 IU/ml) in a CO₂ incubator (culture medium). Same day as activation, T cells were transduced with lentiviral particle containing anti-FAP CAR expressed under the control of an EF1A promoter (SEQ ID NO: 113 (Table 2, CLSFAP1-CAR)) at an MOI of 10.

Four days after transduction, FAP-CAR-T cells were electroporated with 5 μg of each TALEN® arm mRNAs specific for TRAC (SEQ ID NO: 74 and SEQ ID NO: 75) or PDCD1 (SEQ ID NO: 72 and SEQ ID NO: 73). Transfection was performed using Pulse Agile technology by applying two 0.1 mS pulses at 800V followed by four 0.2 mS pulses at 130 V in 0.4 cm gap cuvettes in Cytoporation buffer T (BTX Harvard Apparatus, Holliston, Massachusetts). The electroporated cells were then immediately transferred into prewarmed Optmizer serum-free media and incubated at 37° C. for 15 min. The cells were then concentrated and incubated in the presence of PD1-Meso-CAR AAV particles (MOI=7.5E4 vg/cells), comprising the donor matrices depicted in FIG. 2C. After 2 h of culture at 30° C., OpTmizer media supplemented by 10% AB serum and IL-2 was added to the cell suspension, and the mix was incubated for 16 h under the same culture conditions. Cells were subsequently cultivated at 37° C. in the presence of 5% CO₂.

### Enrichment of PD1-MesoCAR-T Cells by Magnetic Sorting of DLNGFR Positive Cells

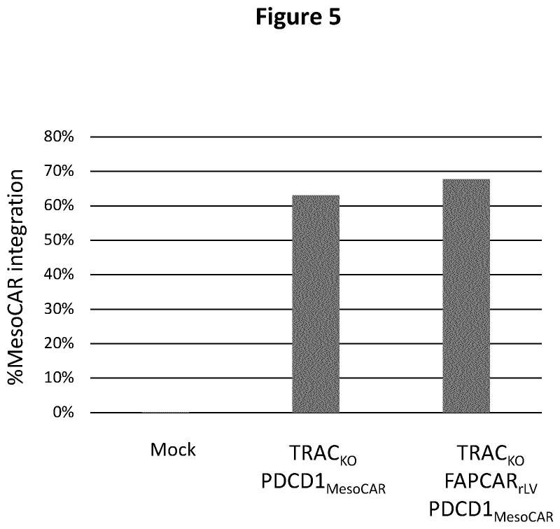

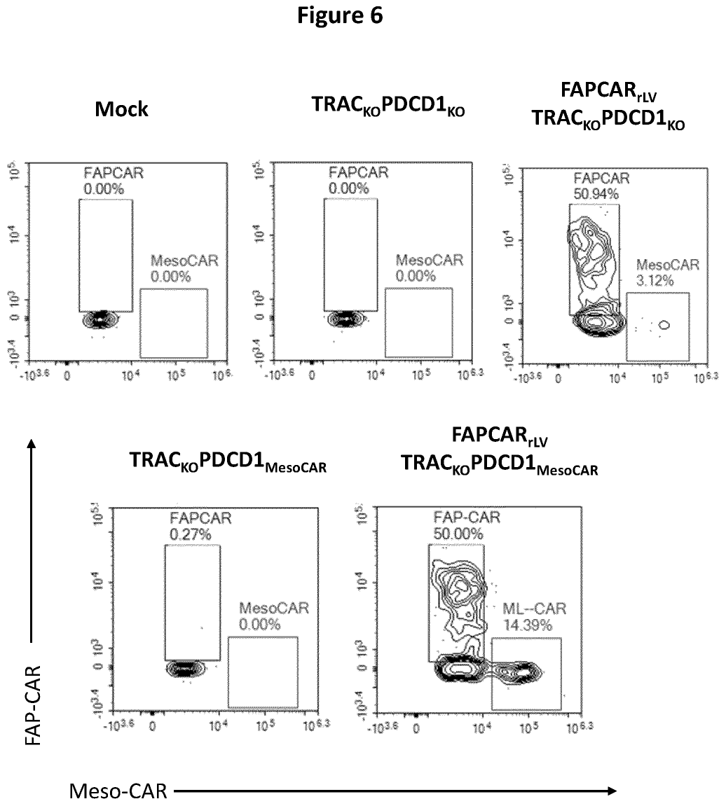

As described above, cells with integration of the Meso-CAR matrix at PDCD1 locus express a non-functional truncated LNGFR (DLNGFR) protein on their surface. DLNGFR positive T cells were positively selected by magnetic sorting using MACSelect™ LNGFR MicroBeads, as per manufacturer protocol (Miltenyi). Positive selection of DLNGFR+ cells led to significant enrichment of PDCD1-integrated Meso-CAR-T cells, as measured by ddPCR (FIG. 5). Subsequently, these cells were analysed for TRAC knockout, FAP-CAR expression and Meso-CAR expression 24 h post activation by overnight incubation on 12-well tissue culture plates coated with 1 mg/ml FAP protein (LakePharma) (FIG. 6). Importantly no Meso-CAR could be detected while its sequence is targeted at the PDCD1 locus but without the presence of the FAP-CAR. Whereas, when FAP-CAR is constitutively expressed, FAP protein could induce Meso-CAR expression in 14% of the edited T-cells. This clearly demonstrates a tight control of Meso-CAR expression upon FAP activation. Surprisingly, the FAP-CAR and the Meso-CAR T cells were not expressed together.

### Example 4: Cytotoxic Activity of CAR-T Cells Having a Constitutive Expression of a FAP-CAR and an Inducible Expression of a Meso-CAR

This example demonstrates that combining a constitutive expression of a FAP-CAR with an inducible expression of tumor antigen targeting-CAR enhances the specific killing of tumor cells. Enriched engineered T-cells produced in Example 2 were used.

### Tumor-CAF Spheroid Seeding

A 3-dimensional spheroid model of mesothelin-expressing triple-negative breast cancer (TNBC) cells and TNBC-derived FAP-expressing CAF cells was established. This model allows to mimic the tumor microenvironment, including the spatial organization and properties of an actual tumor. 104 mesothelin-expressing HCC70 cells, transduced to express GFP and reporter gene Nanoluciferase (HCC70-NL-GFP) were seeded with TNBC-derived CAFs at a 2:1 ratio on low adherence 96-well round bottom plates, in DMEM+10% FBS media. Under these conditions, tumor cells and CAF cells organize themselves into spheroids mimicking in vivo tumor properties.

Cytolytic Activity of TRAC_{FAPCAR}PDCD1_{MesoCAR} T Cells Against Tumor-CAF Spheroids

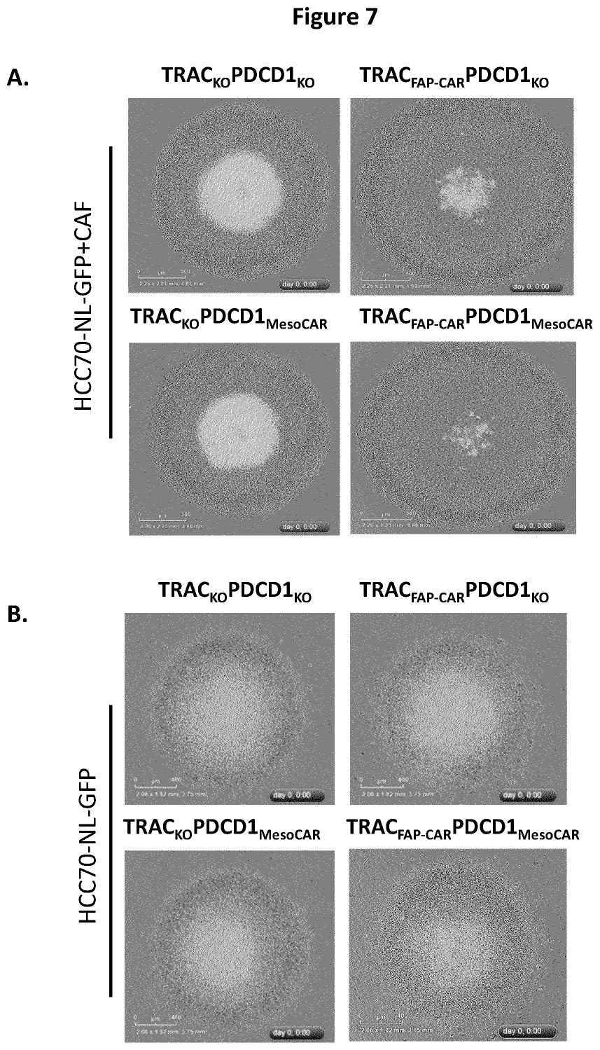

Cytolytic activity of TRAC_{FAPCAR}PDCD1_{MesoCAR} T-cells (as described in Example 2) against HCC70-NL-GFP tumor cells in tumor-CAF spheroids was determined by adding TRAC_{FAPCAR}PDCD1_{MesoCAR} to spheroids plated as described above, two days after spheroid seeding, at tumor cell: CAR-T ratio of 1:2. TRAC_{KO}PDCD1_{KO}, TRAC_{FAPCAR}PDCD1_{KO} and TRAC_{KO}PDCD1_{MesoCAR} were used as controls. 72 h post co-incubation with the different edited T-cells, HCC70-NL-GFP lysis was determined by imaging the spheroids on Incucyte ZOOM and analysed for GFP expression (FIG. 7). The assay showed i) improved activity with TRAC_{KO}PDCD1_{MesoCAR} compared to TRAC_{FAPCAR}PDCD1_{KO}, and ii) no activity of TRAC_{KO}PDCD1_{MesoCAR} or TRAC_{KO}PDCD1_{KO}, demonstrating a tight CAR-inducible system. Finally, the highest killing was observed with the TRAC_{FAPCAR}PDCD1_{MesoCAR} (FIG. 7A).

Furthermore, minimal killing of HCC70-NL-GFP alone spheroids was observed with both TRAC_{FAPCAR}PDCD1_{KO} and TRAC_{FAPCAR}PDCD1_{MesoCAR} T cells (FIG. 7B), indicating that tumor cells killing observed in presence of CAFs is mediated by Meso-CAR. Therefore, the presence of FAP⁺ CAFs is necessary for expression and activity of Meso-CAR. Thus, this strategy will allow the anti-Mesothelin CAR-T activity to localize only to tumors comprising FAP⁺ CAFs, thus reducing ‘on target, off tumor’ cytotoxicity. Additionally, since CAFs are known to be tumor promoting, CAF cytotoxicity combined with tumor cell killing is a strategy that will rapidly inhibit tumor promotion.

### Example 5: Safety Measurement In Vivo

To demonstrate increased anti-tumor activity with simultaneous decrease in ‘on target, off tumor’ cytotoxicity of this strategy in vivo, immune compromised NSG mice will be sub-cutaneously implanted with human mesothelioma cell line NCI-H226 (“NCI-H226 tumor alone”) on the left flank, and NCI-H226 cells mixed with FAP protein expressing NCI-H226 cells (“NCI-H226-FAP”) at a 4:1 ratio. NCI-H226 tumors alone in this system mimic an ‘on target, off-tumor’ site. Mice are injected with CAR-T cells engineered as in Example 1 or 2. TRAC_{FAPCAR}PDCD1_{MesoCAR} T cells show maximum cytotoxic activity against the NCI-H226-FAP tumors, while displaying minimal killing activity against NCI-H226 tumors alone within the same mice.

### Example 6: Identification of Some Promoters Inducible Upon CAR-T Cells Activation

Anti-CS1-CART cells were produced using an 18-day process as briefly described below.

Human PBMCs were thawed and activated using TransAct beads. 3 days later, the cells were electroporated with mRNA encoding TRAC-(SEQ ID NO: 74 and SEQ ID NO: 75) and CS1-(SEQ ID NO: 82 and SEQ ID NO: 83) specific TALE-Nucleases. 2 days later, the cells were transduced with a lentiviral vector driving expression of CS1-specific second generation CAR (SEQ ID NO: 96), followed by an in vitro expansion phase and magnetic depletion of remaining alpha/betaTCR-positive cells. At the end of the production process, the CAR-T cells were filled into vials and stored frozen.

A fraction of CS1-CAR-T cells were thawed and CAR+ T cells were sorted by flow-activated cell sorting (FACS) using CAR-specific reagent (“unactivated cell sample”).

In parallel, another fraction of CS1-CAR-T cells were thawed and activated using plate-bound CS1 recombinant protein (SEQ ID NO: 97). 24 h after activation, CAR+ T cells were FACS-sorted (“activated cell sample”).

Unactivated and activated samples from 2 independent donors were analyzed by RNA-seq.

To identify activation induced genes, genes were selected that fulfill the following criteria:
maximum expression level at 0 h is lower than 100 TPM (transcripts per kilobase million);
minimum expression level at 24 h is greater than 50 TPM; and
fold change between average expression at 0 h and average expression at 24 h is greater than 5.

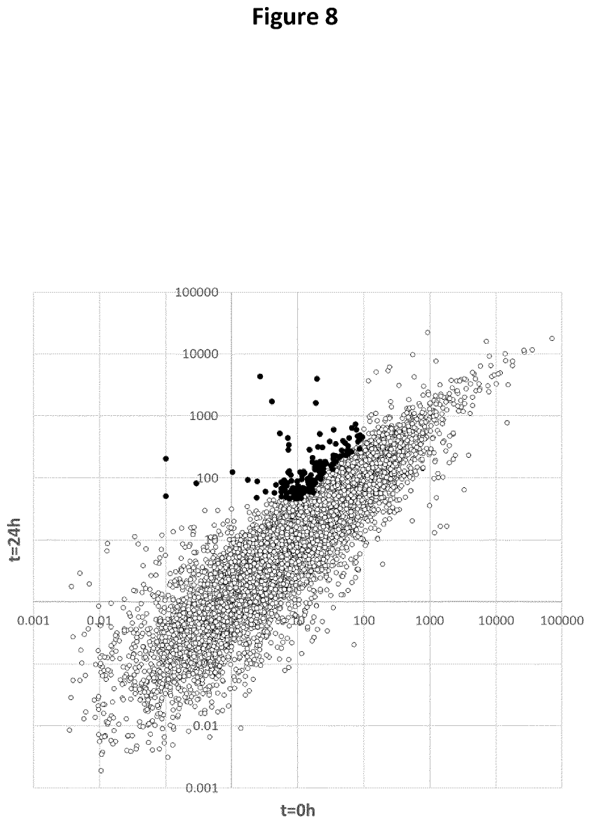

These criteria led to the identification of 159 genes (represented in FIG. 8). From this list, based on literature, we identified the ones that adversely affect T cell proliferation, tumor infiltration or function. Some of those are presented in Table 7.

TABLE 7
Examples of genes induced upon T-cell activation

| Symbol | Gene Accession Number |
|---|---|
| PDCD1 | ENSG00000188389 |
| CCL1 | ENSG00000108702 |
| NR4A3 | ENSG00000119508 |
| EGR3 | ENSG00000179388 |
| G0S2 | ENSG00000123689 |
| IL22 | ENSG00000127318 |
| RGS16 | ENSG00000143333 |
| FASLG | ENSG00000117560 |
| RDH10 | ENSG00000121039 |
| CSF1 | ENSG00000184371 |

### Example 7: Measurement of ‘on Tumor’ and Bystander Cytolytic Activity of FAP-CAR; TRAC_{KO}PDCD1_{Meso-CAR} T-Cells In Vivo

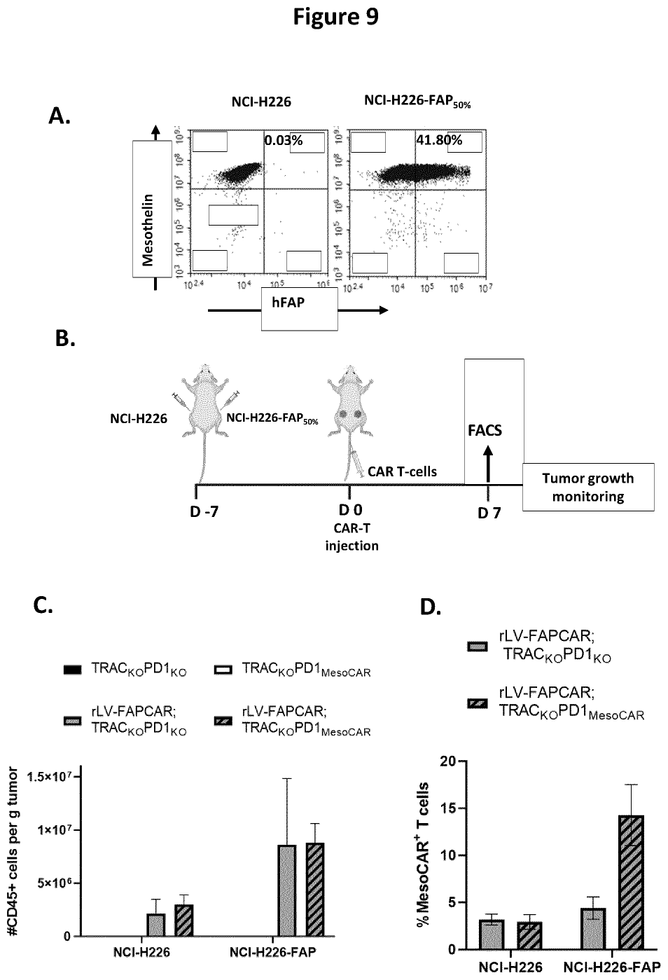

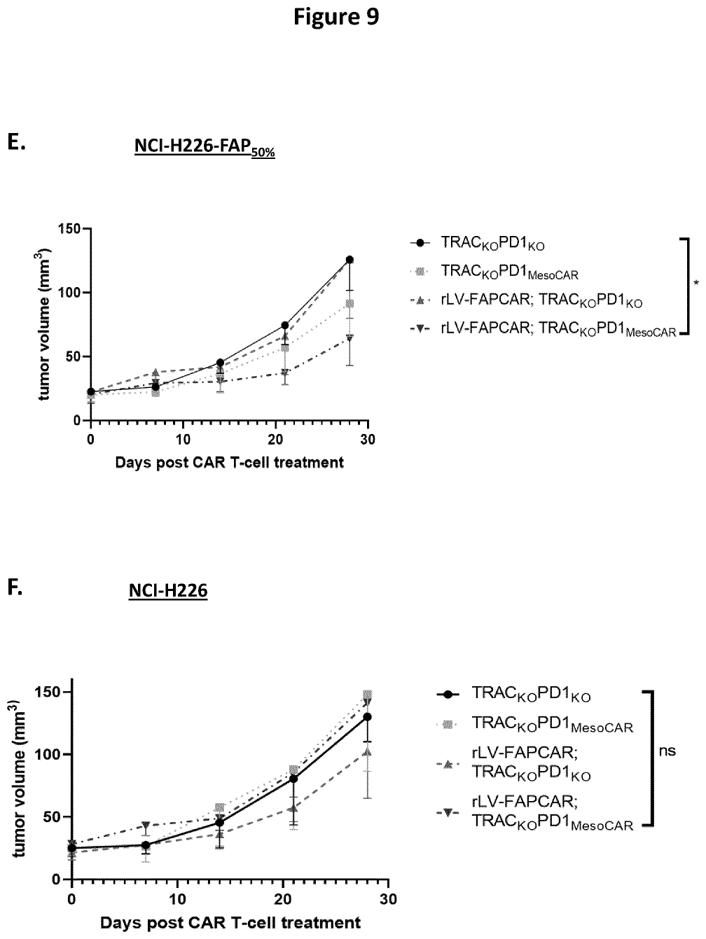

To assess the in vivo activity and safety of our inducible dual CAR T-cell approach, we developed a murine bilateral tumor model with a FAP and mesothelin double-positive ‘on tumor’ site and a FAP-negative, mesothelin-positive ‘off tumor’ site. Immune compromised NSG mice were first sub-cutaneously implanted with 5×10⁶ human mesothelioma cell line NCI-H226 expressing the tumor antigen Mesothelin (“NCI-H226 tumor”) on the left flank, and 5×10⁶ NCI-H226 expressing the tumor antigen Mesothelin and FAP protein at 50% cellular frequency on the right flank (“NCI-H226-FAP_{50%} tumor”) (FIG. 9A). “NCI-H226 tumor” in this system mimics an ‘on target, off-tumor’ site.

Mice were then injected intra-venously with 15×10⁶ CAR⁺ T-cells engineered as in Example 1 or 2 (FIG. 9B). Seven days later (D7), a subset of these mice was euthanised, tumors were excised and processed to single-cell suspension by digestion with Accutase at 37° C. for 15 minutes. Flow cytometry analysis of the tumor cell suspension revealed significant accumulation of CD45⁺ cells in both NCI-H226 and NCI-H226-FAP_{50%} tumors of mice treated with FAP-CAR; TRAC_{KO}PDCD1_{KO} and FAP-CAR; TRAC_{KO}PDCD1_{Meso-CAR} T-cells (FIG. 9C). Notably, there were much higher CD45⁺ cells in the NCI-H226-FAP_{50%} tumors in both treatment groups, indicative of antigen-driven expansion. These CD45⁺ cells were further analysed for expression of the Mesothelin-CAR. As expected, Meso-CAR expression was significantly detected only in CD45⁺ cells in NCI-H226-FAP_{50%} tumors of mice treated with FAP-CAR; TRAC_{KO}PDCD1_{Meso-CAR} T-cells, relative to background expression in NCI-H226-FAP_{50%} tumors of mice treated with FAP-CAR; TRAC_{KO}PDCD1_{KO} (FIG. 9D). Meso-CAR expression in CD45⁺ cells in NCI-H226 tumors of mice treated with FAP-CAR; TRAC_{KO}PDCD1_{Meso-CAR} T-cells was same as the background, thus confirming the tight regulation of FAP-CAR-induced Meso-CAR expression.

The remaining mice cohorts were monitored for three more weeks for tumor growth. During the course of the study, only treatment with FAP-CAR; TRAC_{KO}PDCD1_{Meso-CAR} T-cells resulted in significant regression of the NCI-H226-FAP 50% tumors, indicating their increased cytotoxicity due to co-expression of both the FAP-CAR and the Meso-CAR within these tumors (FIG. 9E). Furthermore, NCI-H226 tumors in the same FAP-CAR; TRAC_{KO}PDCD1_{Meso-CAR} T-cell treated mice did not show any growth reduction, relative to all other treatment groups (FIG. 9F). Taken together, our study demonstrates the enhanced cytotoxic activity of dual inducible FAP-CAR; TRAC_{KO}PDCD1_{Meso-CAR} T-cells against the ‘on target’ NCI-H226-FAP tumors, while validating their safety as observed by minimal bystander killing of the FAP-negative, mesothelin-positive NCI-H226 tumors within the same mice.

1.-50. (canceled)

51. An engineered immune cell comprising:
a) an exogenous nucleic acid sequence encoding a chimeric antigen receptor (CAR) targeting a Fibroblast Activation Protein (FAP) (“FAP-CAR”) placed under the transcriptional control of a constitutive promoter; and
b) an exogenous nucleic acid sequence encoding a chimeric antigen receptor (CAR) targeting a tumor antigen (“tumor-CAR”) placed under the transcriptional control of an inducible promoter;
wherein said exogenous nucleic acid sequences of a) and b) are integrated in the cell's genome, and wherein the expression of the tumor-CAR is inducible upon activation of the immune cell.

52. The immune cell according to claim 51, wherein:
the constitutive promoter of a) is selected from the group consisting of an EF1A promoter, a CD52 promoter, a GAPDH promoter, a CMV promoter, an hPGK promoter, a UBC promoter, a SV40 promoter, a PGK promoter, a CAGG promoter, a TRAC promoter, a TRBC promoter, a TRGC promoter, a TRDC promoter, a B2M promoter, a CD5 promoter, a CS1 promoter, a CD45 promoter, a RPBSA promoter, a CD4 promoter, and a CD8 promoter; and/or
the inducible promoter of b) is selected from the group consisting of a PDCD1 promoter, a CD25 promoter, a TIM3 promoter, a TIGIT promoter, a CCL1 promoter, a NR4A3 promoter, an EGR3 promoter, a GOS2 promoter, an IL22 promoter, a RGS16 promoter, a FASLG promoter, a RDH10 promoter, a CSF1 promoter, a GM-CSF promoter, a LAG3 promoter, a CTLA-4 promoter, an IL10 promoter, a NUR77 promoter, a FOXP3 promoter, and a NFAT responsive element.

53. The immune cell according to claim 51, wherein the immune cell is a primary immune cell selected from a macrophage, a Natural Killer-cell, a T-cell, an inflammatory T-lymphocyte, a cytotoxic T-lymphocyte, and a helper T-lymphocyte.

54. The immune cell according to claim 51, wherein the immune cell has been genetically modified:
(i) to suppress or repress expression of one or more of:
at least one gene encoding a component of a T-Cell Receptor (TCR) selected from a TCRα gene, a TCRβ gene, or a TCRα gene and a TCRβ gene,
at least one gene encoding a MHC-I protein selected from β2m and HLA,
a gene encoding an immune checkpoint protein and/or a receptor thereof,
at least one immune suppressive or chemotherapy drug; and/or
(ii) to contain a suicide gene.

55. The immune cell according to claim 51, wherein the immune cell is one or more of: TCR negative, B2M negative, PDCD1 negative, and CD52 negative.

56. The immune cell according to claim 51, wherein the tumor antigen targeted by said tumor-CAR is an antigen present in a solid tumor or in a haematological cancer, wherein said tumor or cancer is characterized by the presence of FAP in said tumor's or cancer's microenvironment.

57. The immune cell according to claim 51, wherein said tumor antigen is an antigen present both on solid tumors and on some normal healthy tissues, selected from CEA, ERBB2, EGFR, GD2, mesothelin, MUC1, PSMA, GD2, PSMA1, LAP3, ANXA3, TAG72, MUC16, 5T4, FRα, MUC28z, NKG2D, HRG1β, PSCA, PSMA, CA-IX, Trop2, claudin18.2, FOLR1, CXCR2, B7-H3, CD133, CD24, ROR1, EGFR, EGFRvIII, VEGF, EphA2, DLL3, glypican-3, EpCAM, GUCY2C, DCLK1, HER receptors HER1, HER2, HER3, HER4, PEM, A33, G250, carbohydrate antigens Le^{y}, Le^{x}, Le^{b}, STEAP1, CD166, CD24, CD44, E-cadherin, SPARC, and ErbB3.

58. The immune cell according to claim 51, wherein:
said FAP-CAR comprises:
(a1) an extracellular FAP-binding-domain comprising VH and VL amino acid sequences from a monoclonal anti-FAP antibody,
(b1) a hinge selected from a FcγRIII hinge, a CD8α hinge, and an IgG1 hinge,
(c1) a transmembrane domain comprising a CD8α transmembrane domain or a CD28 transmembrane domain, and
(d1) a cytoplasmic domain comprising (i) a CD3 zeta signaling domain, and optionally (ii) a co-stimulatory domain from 4-1BB or from CD28; and/or
said tumor-CAR comprises:
(a2) an extracellular tumor antigen-binding-domain comprising VH and VL amino acid sequences from a monoclonal anti-tumor antigen antibody,
(b2) a hinge selected from a FcγRIII hinge, a CD8α hinge, and an IgG1 hinge,
(c2) a transmembrane domain comprising a CD8α transmembrane domain or a CD28 transmembrane domain, and
(d2) a cytoplasmic domain comprising (i) a CD3 zeta signaling domain and (ii) a co-stimulatory domain from 4-1BB or from CD28.

59. The immune cell according to claim 51, wherein said FAP-CAR comprises an extracellular FAP-binding-domain comprising:
(a) a Variable Heavy Chain (VH) comprising an amino acid sequence having at least 80% identity with VH of amino acid sequence SEQ ID NO: 7 and comprising the H-CDRs of SEQ ID NO: 1, SEQ ID NO: 2, and SEQ ID NO: 3, and a Variable Light Chain (VL) comprising an amino acid sequence having at least 80% identity with VL of amino acid sequence SEQ ID NO: 8 and comprising the L-CDRs of SEQ ID NO: 4, SEQ ID NO: 5, and SEQ ID NO: 6;
(b) a Variable Heavy Chain (VH) comprising an amino acid sequence having at least 80% identity with VH of amino acid sequence SEQ ID NO: 18 and comprising the H-CDRs of SEQ ID NO: 12, SEQ ID NO: 13, and SEQ ID NO: 14, and a Variable Light Chain (VL) comprising an amino acid sequence having at least 80% identity with VL of amino acid sequence SEQ ID NO: 19 and comprising the L-CDRs of SEQ ID NO: 15, SEQ ID NO: 16, and SEQ ID NO: 17;
(c) a Variable Heavy Chain (VH) comprising an amino acid sequence having at least 80% identity with VH of amino acid sequence SEQ ID NO: 29 and comprising the H-CDRs of SEQ ID NO: 23, SEQ ID NO: 24, and SEQ ID NO: 25, and a Variable Light Chain (VL) comprising an amino acid sequence having at least 80% identity with VL of amino acid sequence SEQ ID NO: 30 and comprising the L-CDRs of SEQ ID NO: 26, SEQ ID NO: 27, and SEQ ID NO: 28; or
(d) a Variable Heavy Chain (VH) comprising an amino acid sequence having at least 80% identity with VH of amino acid sequence SEQ ID NO: 40 and comprising the H-CDRs of SEQ ID NO: 34, SEQ ID NO: 35, and SEQ ID NO: 36, and a Variable Light Chain (VL) comprising an amino acid sequence having at least 80% identity with VL of amino acid sequence SEQ ID NO: 41 and comprising the L-CDRs of SEQ ID NO: 37, SEQ ID NO: 38, and SEQ ID NO: 39.

60. The immune cell according to claim 51, wherein said tumor-CAR comprises an extracellular binding-domain targeting a tumor antigen selected from the group consisting of CEA, ERBB2, EGFR, GD2, mesothelin, MUC1, PSMA, GD2, PSMA1, LAP3, ANXA3, TAG72, MUC16, 5T4, FRα, MUC28z, NKG2D, HRG1β, PSCA, PSMA, CA-IX, Trop2, claudin18.2, FOLR1, CXCR2, B7-H3, CD133, CD24, ROR1, EGFR, EGFRvIII, VEGF, EphA2, DLL3, glypican-3, EpCAM, GUCY2C, DCLK1, HER receptors HER1, HER2, HER3, HER4, PEM, A33, G250, carbohydrate antigens Le^{y}, Le^{x}, Le^{b}, STEAP1, CD166, CD24, CD44, E-cadherin, SPARC, and ErbB3.

61. The immune cell according to claim 51, wherein said tumor-CAR comprises an extracellular binding-domain comprising:
a) the H-CDRs of SEQ ID NO: 47, SEQ ID NO: 48, and SEQ ID NO: 49, and the L-CDRs of SEQ ID NO: 50, SEQ ID NO: 51, and SEQ ID NO: 52, and an amino acid sequence having at least 80% identity with the amino acid sequence set forth in SEQ ID NO: 53;
b) the H-CDRs of SEQ ID NO: 55, SEQ ID NO: 56, and SEQ ID NO: 57, and the L-CDRs of SEQ ID NO: 58, SEQ ID NO: 59, and SEQ ID NO: 60, and an amino acid sequence having at least 80% identity with the amino acid sequence set forth in SEQ ID NO: 61;
c) the H-CDRs of SEQ ID NO: 63, SEQ ID NO: 64, and SEQ ID NO: 65, and the L-CDRs of SEQ ID NO: 66, SEQ ID NO: 67, and SEQ ID NO: 68, and an amino acid sequence having at least 80% identity with the amino acid sequence set forth in SEQ ID NO: 69; or
d) the H-CDRs and the L-CDRs comprised in the amino acid sequence of SEQ ID NO: 71, and an amino acid sequence having at least 80% identity with the amino acid sequence set forth in SEQ ID NO: 71.

62. A pharmaceutical composition comprising a therapeutically effective amount of immune cells according to claim 51.

63. A method of treatment of a cancer characterized by the presence of FAP in the tumor microenvironment comprising administering a therapeutically effective amount of immune cells according to claim 51.

64. The method of treatment according to claim 63, wherein said cancer is a solid tumor or an haematological cancer selected from the group consisting of breast cancer, ovarian cancer, endometrial cancer, cervical cancer, bladder cancer, renal cancer, melanoma, lung cancer, prostate cancer, testicular cancer, thyroid cancer, brain cancer, esophageal cancer, gastric cancer, pancreatic cancer, colorectal cancer, liver cancer, myelofibrosis, myelodysplastic syndromes, acute myeloid leukemia, non-Hodgkin's lymphoma, and multiple myeloma.

65. A method of producing a population of cells comprising immune cells according to claim 51 comprising:
(i) providing immune cells from a donor or induced pluripotent stem cells (iPSCs);
(ii) optionally, inactivating the potential expression of a T-Cell Receptor (TCR) in the cells or its presentation at the cells' surface;
(iii) integrating in the cells' genome an exogenous nucleic acid sequence encoding a chimeric antigen receptor (CAR) targeting the Fibroblast Activation Protein (FAP) (“FAP-CAR”) placed under the transcriptional control of a constitutive promoter;
(iv) integrating in the cells' genome an exogenous nucleic acid sequence encoding a chimeric antigen receptor (CAR) targeting a tumor antigen (“tumor-CAR”) placed under the transcriptional control of an inducible promoter; and
(v) optionally, isolating the engineered cells that do not express a TCR at their cell surface.

66. The method according to claim 65, wherein said integration is operated through random integration such as through lentiviral vector integration or through gene targeting integration such as through nuclease-mediated cDNA insertion at one targeted gene locus in the cells' genome.

67. The method according to claim 65, comprising inactivating at least one of the TRAC, B2M, and CD52 loci in the cells' genome.

68. A set of vectors comprising (i) at least one vector comprising an expression cassette comprising an exogenous nucleic acid sequence encoding a FAP-CAR placed under the transcriptional control of a constitutive promoter and (il) at least one vector comprising an expression cassette comprising an exogenous nucleic acid sequence encoding a tumor-CAR placed under the transcriptional control of an inducible promoter.

69. A kit comprising the set of vectors of claim 68 and at least one sequence-specific endonuclease targeting one inducible gene locus selected from PDCD1, CD25, TIM3, TIGIT, CCL1, NR4A3, EGR3, GOS2, IL22, RGS16, FASLG, RDH10, CSF1, GM-CSF, LAG3, CTLA-4, IL10, NUR77, and FOXP3 gene loci.


SEQUENCE LISTING (115 sequences)

SEQ ID NO: 1 - protein, 9 aa, synthetic construct
  REGION 1..9: note = H-CDR1
YTFTEYTIH

SEQ ID NO: 2 - protein, 15 aa, synthetic construct
  REGION 1..15: note = H-CDR2
GINPNNGIPNYNQKF

SEQ ID NO: 3 - protein, 15 aa, synthetic construct
  REGION 1..15: note = H-CDR3
RRIAYGYDEGHAMDY

SEQ ID NO: 4 - protein, 14 aa, synthetic construct
  REGION 1..14: note = L-CDR1
QSLLYSRNQKNYLA

SEQ ID NO: 5 - protein, 11 aa, synthetic construct
  REGION 1..11: note = L-CDR2
LLIFWASTRES

SEQ ID NO: 6 - protein, 9 aa, synthetic construct
  REGION 1..9: note = L-CDR3
QQYFSYPLT

SEQ ID NO: 7 - protein, 124 aa, synthetic construct
  REGION 1..124: note = VH
QVQLVQSGAEVKKPGASVKVSCKTSRYTFTEYTIHWVRQAPGQRLEWIGGINPNNGIPNYNQKFKGRVTITVDTSASTAYMELSSLRSEDTAVYYCARRRIAYGYDEGHAMDYWGQGTLVTVSS

SEQ ID NO: 8 - protein, 112 aa, synthetic construct
  REGION 1..112: note = VL
DIVMTQSPDSLAVSLGERATINCKSSQSLLYSRNQKNYLAWYQQKPGQPPKLLIFWASTRESGVPDRFSGSGFGTDFTLTISSLQAEDVAVYYCQQYFSYPLTFGQGTKVEI

SEQ ID NO: 9 - protein, 253 aa, synthetic construct
  REGION 1..253: note = scFv
QVQLVQSGAEVKKPGASVKVSCKTSRYTFTEYTIHWVRQAPGQRLEWIGGINPNNGIPNYNQKFKGRVTITVDTSASTAYMELSSLRSEDTAVYYCARRRIAYGYDEGHAMDYWGQGTLVTVSSGSTSGSGKPGSGEGSTKDIVMTQSPDSLAVSLGERATINCKSSQSLLYSRNQKNYLAWYQQKPGQPPKLLIFWASTRESGVPDRFSGSGFGTDFTLTISSLQAEDVAVYYCQQYFSYPLTFGQGTKVEI

SEQ ID NO: 10 - protein, 511 aa, synthetic construct
  REGION 1..511: note = FAP1-41BB-CAR
MALPVTALLLPLALLLHAARPQVQLVQSGAEVKKPGASVKVSCKTSRYTFTEYTIHWVRQAPGQRLEWIGGINPNNGIPNYNQKFKGRVTITVDTSASTAYMELSSLRSEDTAVYYCARRRIAYGYDEGHAMDYWGQGTLVTVSSGSTSGSGKPGSGEGSTKDIVMTQSPDSLAVSLGERATINCKSSQSLLYSRNQKNYLAWYQQKPGQPPKLLIFWASTRESGVPDRFSGSGFGTDFTLTISSLQAEDVAVYYCQQYFSYPLTFGQGTKVEIFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - protein, 499 aa, synthetic construct
  REGION 1..499: note = FAP1-CD28-CAR
MALPVTALLLPLALLLHAARPQVQLVQSGAEVKKPGASVKVSCKTSRYTFTEYTIHWVRQAPGQRLEWIGGINPNNGIPNYNQKFKGRVTITVDTSASTAYMELSSLRSEDTAVYYCARRRIAYGYDEGHAMDYWGQGTLVTVSSGSTSGSGKPGSGEGSTKDIVMTQSPDSLAVSLGERATINCKSSQSLLYSRNQKNYLAWYQQKPGQPPKLLIFWASTRESGVPDRFSGSGFGTDFTLTISSLQAEDVAVYYCQQYFSYPLTFGQGTKVEITTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 12 - protein, 9 aa, synthetic construct
  REGION 1..9: note = H-CDR1
YTFTDYYMK

SEQ ID NO: 13 - protein, 15 aa, synthetic construct
  REGION 1..15: note = H-CDR2
DIYPNNGEIPYNQKF

SEQ ID NO: 14 - protein, 12 aa, synthetic construct
  REGION 1..12: note = H-CDR3
VRGYYYGLAMDY

SEQ ID NO: 15 - protein, 11 aa, synthetic construct
  REGION 1..11: note = L-CDR1
TGAVTTSNYAN

SEQ ID NO: 16 - protein, 11 aa, synthetic construct
  REGION 1..11: note = L-CDR2
GLIGATNNRAP

SEQ ID NO: 17 - protein, 9 aa, synthetic construct
  REGION 1..9: note = L-CDR3
ALWYSNHFI

SEQ ID NO: 18 - protein, 119 aa, synthetic construct
  REGION 1..119: note = VH
EVQLQQSGPELVKPGASVRMSCKASGYTFTDYYMKWVKQSLGKSLEWIGDIYPNNGEIPYNQKFKGKATLTADKTSSTAYMQLNSLTSEDSAVYYCVRGYYYGLAMDYWGQGTSVTSVV

SEQ ID NO: 19 - protein, 108 aa, synthetic construct
  REGION 1..108: note = VL
QAVVTQESALTSPGETVTLTCRSSTGAVTTSNYANWVQEKPDRLFTGLIGATNNRAPGVPARFSGSLIGDKAALTITGAQTEDEAIYFCALWYSNHFIFGSGTKVTVL

SEQ ID NO: 20 - protein, 242 aa, synthetic construct
  REGION 1..242: note = scFv
EVQLQQSGPELVKPGASVRMSCKASGYTFTDYYMKWVKQSLGKSLEWIGDIYPNNGEIPYNQKFKGKATLTADKTSSTAYMQLNSLTSEDSAVYYCVRGYYYGLAMDYWGQGTSVTSVVGGGGSGGGGSGGGGSQAVVTQESALTSPGETVTLTCRSSTGAVTTSNYANWVQEKPDRLFTGLIGATNNRAPGVPARFSGSLIGDKAALTITGAQTEDEAIYFCALWYSNHFIFGSGTKVTVL

SEQ ID NO: 21 - protein, 515 aa, synthetic construct
  REGION 1..515: note = FAP2-41BB-CAR
MALPVTALLLPLALLLHAARPEVQLQQSGPELVKPGASVRMSCKASGYTFTDYYMKWVKQSLGKSLEWIGDIYPNNGEIPYNQKFKGKATLTADKTSSTAYMQLNSLTSEDSAVYYCVRGYYYGLAMDYWGQGTSVTSVVGGGGSGGGGSGGGGSQAVVTQESALTSPGETVTLTCRSSTGAVTTSNYANWVQEKPDRLFTGLIGATNNRAPGVPARFSGSLIGDKAALTITGAQTEDEAIYFCALWYSNHFIFGSGTKVTVLGGGGSGGGGSGGGGSFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - protein, 495 aa, synthetic construct
  REGION 1..495: note = FAP2-CD28-CAR
MALPVTALLLPLALLLHAARPEVQLQQSGPELVKPGASVRMSCKASGYTFTDYYMKWVKQSLGKSLEWIGDIYPNNGEIPYNQKFKGKATLTADKTSSTAYMQLNSLTSEDSAVYYCVRGYYYGLAMDYWGQGTSVTSVVGGGGSGGGGSGGGGSQAVVTQESALTSPGETVTLTCRSSTGAVTTSNYANWVQEKPDRLFTGLIGATNNRAPGVPARFSGSLIGDKAALTITGAQTEDEAIYFCALWYSNHFIFGGGGGSGGGGSGGGGSTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 23 - protein, 9 aa, synthetic construct
  REGION 1..9: note = H-CDR1
YTFTENIIH

SEQ ID NO: 24 - protein, 15 aa, synthetic construct
  REGION 1..15: note = H-CDR2
WFHPGSGSIKYNEKF

SEQ ID NO: 25 - protein, 11 aa, synthetic construct
  REGION 1..11: note = H-CDR3
HGGTGRGAMDY

SEQ ID NO: 26 - protein, 12 aa, synthetic construct
  REGION 1..12: note = L-CDR1
KSVSTSAYSYMH

SEQ ID NO: 27 - protein, 11 aa, synthetic construct
  REGION 1..11: note = L-CDR2
LLIYLASNLES

SEQ ID NO: 28 - protein, 9 aa, synthetic construct
  REGION 1..9: note = L-CDR3
QHSRELPYT

SEQ ID NO: 29 - protein, 120 aa, synthetic construct
  REGION 1..120: note = VH
QVQLVQSGAEVKKPGASVKVSCKASGYTFTENIIHWVRQAPGQGLEWMGWFHPGSGSIKYNEKFKDRVTMTADTSTSTVYMELSSLRSEDTAVYYCARHGGTGRGAMDYWGQGTLVTVSS

SEQ ID NO: 30 - protein, 112 aa, synthetic construct
  REGION 1..112: note = VL
DIQMTQSPSSLSASVGDRVTITCRASKSVSTSAYSYMHWYQQKPGKAPKLLIYLASNLESGVPSRFSGSGSGTDFTLTISSLQPEDFATYYCQHSRELPYTFGQGTKLEIKR

SEQ ID NO: 31 - protein, 247 aa, synthetic construct
  REGION 1..247: note = scFv
QVQLVQSGAEVKKPGASVKVSCKASGYTFTENIIHWVRQAPGQGLEWMGWFHPGSGSIKYNEKFKDRVTMTADTSTSTVYMELSSLRSEDTAVYYCARHGGTGRGAMDYWGQGTLVTVSSGGGGSGGGGSGGGGSDIQMTQSPSSLSASVGDRVTITCRASKSVSTSAYSYMHWYQQKPGKAPKLLIYLASNLESGVPSRFSGSGSGTDFTLTISSLQPEDFATYYCQHSRELPYTFGQGTKLEIKR

SEQ ID NO: 32 - protein, 520 aa, synthetic construct
  REGION 1..520: note = FAP3-41BB-CAR
MALPVTALLLPLALLLHAARPQVQLVQSGAEVKKPGASVKVSCKASGYTFTENIIHWVRQAPGQGLEWMGWFHPGSGSIKYNEKFKDRVTMTADTSTSTVYMELSSLRSEDTAVYYCARHGGTGRGAMDYWGQGTLVTVSSGGGGSGGGGSGGGGSDIQMTQSPSSLSASVGDRVTITCRASKSVSTSAYSYMHWYQQKPGKAPKLLIYLASNLESGVPSRFSGSGSGTDFTLTISSLQPEDFATYYCQHSRELPYTFGQGTKLEIKRGGGGSGGGGSGGGGSFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 33 - protein, 508 aa, synthetic construct
  REGION 1..508: note = FAP3-CD28-CAR
MALPVTALLLPLALLLHAARPQVQLVQSGAEVKKPGASVKVSCKASGYTFTENIIHWVRQAPGQGLEWMGWFHPGSGSIKYNEKFKDRVTMTADTSTSTVYMELSSLRSEDTAVYYCARHGGTGRGAMDYWGQGTLVTVSSGGGGSGGGGSGGGGSDIQMTQSPSSLSASVGDRVTITCRASKSVSTSAYSYMHWYQQKPGKAPKLLIYLASNLESGVPSRFSGSGSGTDFTLTISSLQPEDFATYYCQHSRELPYTFGQGTKLEIKRGGGGSGGGGSGGGGSTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 34 - protein, 9 aa, synthetic construct
  REGION 1..9: note = H-CDR1
FTFSSYAMS

SEQ ID NO: 35 - protein, 17 aa, synthetic construct
  REGION 1..17: note = H-CDR2
VSAIIGSGASTYYADSV

SEQ ID NO: 36 - protein, 9 aa, synthetic construct
  REGION 1..9: note = H-CDR3
KGWFGGFNY

SEQ ID NO: 37 - protein, 9 aa, synthetic construct
  REGION 1..9: note = L-CDR1
QSVTSSYLA

SEQ ID NO: 38 - protein, 13 aa, synthetic construct
  REGION 1..13: note = L-CDR2
LLINVGSRRATGI

SEQ ID NO: 39 - protein, 9 aa, synthetic construct
  REGION 1..9: note = L-CDR3
QQGIMLPPT

SEQ ID NO: 40 - protein, 117 aa, synthetic construct
  REGION 1..117: note = VH
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSS

SEQ ID NO: 41 - protein, 108 aa, synthetic construct
  REGION 1..108: note = VL
EIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIK

SEQ ID NO: 42 - protein, 242 aa, synthetic construct
  REGION 1..242: note = scFv
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSGSTSGSGKPGSGEGSTKEIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIK

SEQ ID NO: 43 - protein, 513 aa, synthetic construct
  REGION 1..513: note = FAP4-41BB-CAR
MALPVTALLLPLALLLHAARPEVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSGGGGSGGGGSGGGGSGGGGSGGGGSGGGGSEIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIKFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 44 - protein, 488 aa, synthetic construct
  REGION 1..488: note = FAP4-CD28-CAR
MALPVTALLLPLALLLHAARPEVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSGSTSGSGKPGSGEGSTKEIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 45 - protein, 17 aa, synthetic construct
  REGION 1..17: note = Linker1
GSTSGSGKPGSGEGSTK

SEQ ID NO: 46 - protein, 15 aa, synthetic construct
  REGION 1..15: note = Linker2
GGGGSGGGGSGGGGS

SEQ ID NO: 47 - protein, 9 aa, synthetic construct
  REGION 1..9: note = Trop2 H-CDR1
YTFTNYGMN

SEQ ID NO: 48 - protein, 14 aa, synthetic construct
  REGION 1..14: note = Trop2 H-CDR2
MGWINTYTGEPTYT

SEQ ID NO: 49 - protein, 12 aa, synthetic construct
  REGION 1..12: note = Trop2 H-CDR3
GGFGSSYWYFDV

SEQ ID NO: 50 - protein, 8 aa, synthetic construct
  REGION 1..8: note = Trop2 L-CDR1
QDVSIAVA

SEQ ID NO: 51 - protein, 11 aa, synthetic construct
  REGION 1..11: note = Trop2 L-CDR2
LLIYSASYRYT

SEQ ID NO: 52 - protein, 9 aa, synthetic construct
  REGION 1..9: note = Trop2 L-CDR3
QQHYITPLT

SEQ ID NO: 53 - protein, 243 aa, synthetic construct
  REGION 1..243: note = Trop2 ScFv
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKGGGGSGGGGSGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 54 - protein, 480 aa, synthetic construct
  REGION 1..480: note = TROP2-CAR
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKGGGGSGGGGSGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 55 - protein, 5 aa, synthetic construct
  REGION 1..5: note = MUC1 H-CDR1
NYGLS

SEQ ID NO: 56 - protein, 16 aa, synthetic construct
  REGION 1..16: note = MUC1 H-CDR2
ENHPGSGIIYHNEKFR

SEQ ID NO: 57 - protein, 9 aa, synthetic construct
  REGION 1..9: note = MUC1 H-CDR3
SSGTRGFAY

SEQ ID NO: 58 - protein, 16 aa, synthetic construct
  REGION 1..16: note = MUC1 L-CDR1
RSSQSIVHSNGNTYLE

SEQ ID NO: 59 - protein, 11 aa, synthetic construct
  REGION 1..11: note = MUC1 L-CDR2
LLIYKVSNRFS

SEQ ID NO: 60 - protein, 9 aa, synthetic construct
  REGION 1..9: note = MUC1 L-CDR3
FQGSHGPWT

SEQ ID NO: 61 - protein, 285 aa, synthetic construct
  REGION 1..285: note = MUC1 ScFv
MEWIWIFLFILSGTAGVQSQVQLQQSGAELARPGASVKLSCKASGYTFTNYGLSWVKQRTGQGLEWIGENHPGSGIIYHNEKFRGKATLTADKSSSTAYVQLSSLTSEDSAVYFCARSSGTRGFAYWGQGTLVTVSAGGGGSGGGGSGGGGSMKLPVRLLVLMFWIPASSSDVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHGPWTFGGGTKLEIKRA

SEQ ID NO: 62 - protein, 522 aa, synthetic construct
  REGION 1..522: note = MUC1-CAR
MEWIWIFLFILSGTAGVQSQVQLQQSGAELARPGASVKLSCKASGYTFTNYGLSWVKQRTGQGLEWIGENHPGSGIIYHNEKFRGKATLTADKSSSTAYVQLSSLTSEDSAVYFCARSSGTRGFAYWGQGTLVTVSAGGGGSGGGGSGGGGSMKLPVRLLVLMFWIPASSSDVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHGPWTFGGGTKLEIKRAFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 63 - protein, 9 aa, synthetic construct
  REGION 1..9: note = Meso H-CDR1
INNNNYYWT

SEQ ID NO: 64 - protein, 19 aa, synthetic construct
  REGION 1..19: note = Meso H-CDR2
WIGYIYYSGSTFYNPSLKS

SEQ ID NO: 65 - protein, 9 aa, synthetic construct
  REGION 1..9: note = Meso H-CDR3
EDTMTGLDV

SEQ ID NO: 66 - protein, 8 aa, synthetic construct
  REGION 1..8: note = Meso L-CDR1
QSINNYLN

SEQ ID NO: 67 - protein, 11 aa, synthetic construct
  REGION 1..11: note = Meso L-CDR2
LLIYAASSLQS

SEQ ID NO: 68 - protein, 8 aa, synthetic construct
  REGION 1..8: note = Meso L-CDR3
QQTYSNPT

SEQ ID NO: 69 - protein, 240 aa, synthetic construct
  REGION 1..240: note = MESO ScFv1
QVQLQESGPGLVKPSQTLSLTCTVSGGSINNNNYYWTWIRQHPGKGLEWIGYIYYSGSTFYNPSLKSRVTISVDTSKTQFSLKLSSVTAADTAVYYCAREDTMTGLDVWGQGTTVTVSSGGGGSGGGGSGGGGSDIQMTQSPSSLSASVGDRVTITCRASQSINNYLNWYQQKPGKAPTLLIYAASSLQSGVPSRFSGSRSGTDFTLTISSLQPEDFAAYFCQQTYSNPTFGQGTKVEVK

SEQ ID NO: 70 - protein, 528 aa, synthetic construct
  REGION 1..528: note = MESO-CAR
MALPVTALLLPLALLLHAARPQVQLQQPGAELVKPGASMKLSCKASGYTFTSYWMHWVKQRPGQGLEWIGMIHPNSDNTIYYEKFKSKATLTVDKSSSTAYMQLSSLTSEDSAVYYCAIIITPVVPKFDYWGQGTTLTVSSGGGGSGGGGSGGGGSDIVMTQSHQFMSTSVGDRVSVTCKASHDVGTSVAWYQQKPGQSPKLLIYWASTRHTGVPDRFTGSGSGTDFTLTISNVQSEDLADYFCQQYSSYPLTFGAGTKLELKRASDPGSGGGGSCPYSNPSLCSGGGGSCPYSNPSLCSGGGGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 71 - protein, 244 aa, synthetic construct
  REGION 1..244: note = MESO-scFv2
QVQLQQPGAELVKPGASMKLSCKASGYTFTSYWMHWVKQRPGQGLEWIGMIHPNSDNTIYYEKFKSKATLTVDKSSSTAYMQLSSLTSEDSAVYYCAIIITPVVPKFDYWGQGTTLTVSSGGGGSGGGGSGGGGSDIVMTQSHQFMSTSVGDRVSVTCKASHDVGTSVAWYQQKPGQSPKLLIYWASTRHTGVPDRFTGSGSGTDFTLTISNVQSEDLADYFCQQYSSYPLTFGAGTKLELKRA

SEQ ID NO: 72 - protein, 924 aa, synthetic construct
  REGION 1..924: note = PDCD1_T01-L
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASNGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 73 - protein, 925 aa, synthetic construct
  REGION 1..925: note = PDCD1_T01-R
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASKLGGKQALETVQALLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASYKGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 74 - protein, 925 aa, synthetic construct
  REGION 1..925: note = TRAC_T01-L
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNGGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASHDGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 75 - protein, 925 aa, synthetic construct
  REGION 1..925: note = TRAC_T01-R
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASNIGGKQALETVQALLPVLCQAHGLTPEQVVAIASHDGGKQALETVQRLLPVLCQAHGLTPQQVVAIASNNGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 76 - protein, 925 aa, synthetic construct
  REGION 1..925: note = CD52_T01-L
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 77 - protein, 925 aa, synthetic construct
  REGION 1..925: note = CD52_T01-R
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNGGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASHDGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 78 - protein, 925 aa, synthetic construct
  REGION 1..925: note = B2M_T01-L4
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNGGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 79 - protein, 925 aa, synthetic construct
  REGION 1..925: note = B2M_T01-R4
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 80 - protein, 925 aa, synthetic construct
  REGION 1..925: note = CIITA_T01-L
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASHDGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 81 - protein, 925 aa, synthetic construct
  REGION 1..925: note = CIITA_T01-R
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNIGGKQALETVQALLPVLCQAHGLTPQQVVAIASNNGGKQALETVQRLLPVLCQAHGLTPQQVVAIASNNGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNNGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 82 - protein, 902 aa, synthetic construct
  REGION 1..902: note = CS1_L
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNIGGKQALETVQALLPVLCQAHGLTPQQVVAIASNGGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASHDGGKQALETVQRLLPVLCQAHGLTPQQVVAIASNGGGRPALESIVAQLSRPDPSGSGSGGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 83 - protein, 902 aa, synthetic construct
  REGION 1..902: note = CS1_R
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPQQVVAIASNNGGKQALETVQRLLPVLCQAHGLTPEQVVAIASNIGGKQALETVQALLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPEQVVAIASNIGGKQALETVQALLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNNGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGRPALESIVAQLSRPDPSGSGSGGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 84 - protein, 21 aa, synthetic construct
  REGION 1..21: note = CD8alpha signal peptide
MALPVTALLLPLALLLHAARP

SEQ ID NO: 85 - protein, 20 aa, synthetic construct
  REGION 1..20: note = Alternative signal peptide
METDTLLLWVLLLWVPGSTG

SEQ ID NO: 86 - protein, 16 aa, synthetic construct
  REGION 1..16: note = Fcgamma RIIIalpha hinge
GLAVSTISSFFPPGYQ

SEQ ID NO: 87 - protein, 45 aa, synthetic construct
  REGION 1..45: note = CD8alpha hinge
TTTPAPRPPTPAPTIASQPLSLRPEACRPAAGGAVHTRGLDFACD

SEQ ID NO: 88 - protein, 231 aa, synthetic construct
  REGION 1..231: note = IgG1 hinge
EPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 89 - protein, 24 aa, synthetic construct
  REGION 1..24: note = CD8alpha transmembrane domain
IYIWAPLAGTCGVLLLSLVITLYC

SEQ ID NO: 90 - protein, 27 aa, synthetic construct
  REGION 1..27: note = CD28 transmembrane domain
FWVLVVVGGVLACYSLLVTVAFIIFWV

SEQ ID NO: 91 - protein, 27 aa, synthetic construct
  REGION 1..27: note = 4-1BB transmembrane domain
IISFFLALTSTALLFLLFFLTLRFSVV

SEQ ID NO: 92 - protein, 42 aa, synthetic construct
  REGION 1..42: note = 4-1BB co-stimulatory domain
KRGRKKLLYIFKQPFMRPVQTTQEEDGCSCRFPEEEEGGCEL

SEQ ID NO: 93 - protein, 41 aa, synthetic construct
  REGION 1..41: note = CD28 co-stimulatory domain
RSKRSRLLHSDYMNMTPRRPGPTRKHYQPYAPPRDFAAYRS

SEQ ID NO: 94 - protein, 112 aa, synthetic construct
  REGION 1..112: note = CD3alpha signalling domain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 95 - protein, 1427 aa, Bulkholderia cenocepacia
  REGION 1..1427: note = BURC1 Double-stranded DNA deaminase toxin A
MYEAARVTDPIDHTSALAGFLVGAVLGIALIAAVAFATFTCGFGVALLAGMMAGIGAQALLSIGESIGKMFSSQSGNIITGSPDVYVNSLSAAYATLSGVACSKHNPIPLVAQGSTNIFINGRPAARKDDKITCGATIGDGSHDTFFHGGTQTYLPVDDEVPPWLRTATDWAFTLAGLVGGLGGLLKASGGLSRAVLPCAAKFIGGYVLGEAFGRYVAGPAINKAIGGLFGNPIDVTTGRKILLAESETDYVIPSPLPVAIKRFYSSGIDYAGTLGRGWVLPWEIRLHARDGRLWYTDAQGRESGFPMLRAGQAAFSEADQRYLTRTPDGRYILHDLGERYYDFGQYDPESGRIAWVRRVEDQAGQWYQFERDSRGRVTEILTCGGLRAVLDYETVFGRLGTVTLVHEDERRLAVTYGYDENGQLASVTDANGAVVRQFAYTNGLMTSHMNALGFTSSYVWSKIEGEPRVVETHTSEGENWTFEYDVAGRQTRVRHADGRTAHWRFDAQSQIVEYTDLDGAFYRIKYDAVGMPVMLMLPGDRTVMFEYDDAGRIIAETDPLGRTTRTRYDGNSLRPVEVVGPDGGAWRVEYDQQGRVVSNQDSLGRENRYEYPKALTALPSAHFDALGGRKTLEWNSLGKLVGYTDCSGKTTRTSFDAFGRICSRENALGQRITYDVRPTGEPRRVTYPDGSSETFEYDAAGTLVRYIGLGGRVQELLRNARGQLIEAVDPAGRRVQYRYDVEGRLRELQQDHARYTFTYSAGGRLLTETRPDGILRRFEYGEAGELLGLDIVGAPDPHATGNRSVRTIRFERDRMGVLKVQRTPTEVTRYQHDKGDRLVKVERVPTPSGIALGIVPDAVEFEYDKGGRLVAEHGSNGSVIYTLDELDNVVSLGLPHDQTLQMLRYGSGHVHQIRFGDQVVADFERDDLHREVSRTQGRLTQRSGYDPLGRKVWQSAGIDPEMLGRGSGQLWRNYGYDGAGDLIETSDSLRGSTRFSYDPAGRLISRANPLDRKFEEFAWDAAGNLLDDAQRKSRGYVEGNRLLMWQDLRFEYDPFGNLATKRRGANQTQRFTYDGQDRLITVHTQDVRGVVETRFAYDPLGRRIAKTDTAFDLRGMKLRAETKRFVWEGLRLVQEVRETGVSSYVYSPDAPYSPVARADTVMAEALAATVIDSAKRAARIFHFHTDPVGAPQEVTDEAGEVAWAGQYAAWGKVEATNRGVTAARTDQPLRFAGQYADDSTGLHYNTFRFYDPDVGRFINQDPIGLNGGANVYHYAPNPVGWVDPWGLAGSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEGAIPVKRGATGETKVFTGNSNSPKSPTKGGC

SEQ ID NO: 96 - protein, 486 aa, synthetic construct
  REGION 1..486: note = CS1 CAR
MALPVTALLLPLALLLHAARPQVQLQQPGAELVRPGASVKLSCKASGYSFTTYWMNWVKQRPGQGLEWIGMIHPSDSETRLNQKFKDKATLTVDKSSSTAYMQLSSPTSEDSAVYYCARSTMIATRAMDYWGQGTSVTVSSGGGGSGGGGSGGGGSDIVMTQSQKSMSTSVGDRVSITCKASQDVITGVAWYQQKPGQSPKLLIYSASYRYTGVPDRFTGSGSGTDFTFTISNVQAEDLAVYYCQQHYSTPLTFGAGTKLELKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 97 - protein, 453 aa, synthetic construct
  REGION 1..453: note = CS1-Fc
MEWSWVFLFFLSVTTGVHSSGPVKELVGSVGGAVTFPLKSKVKQVDSIVWTFNTTPLVTIQPEGGTIIVTQNRNRERVDFPDGGYSLKLSKLKKNDSGIYYVGIYSSSLQQPSTQEYVLHVYEHLSKPKVTMGLQSNKNGTCVTNLTCCMEHGEEDVIYTWKALGQAANESHNGSILPISWRWGESDMTFICVARNPVSRNFSSPILARKLCEGAADDPDSSMGGGGAGGGGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 98 - DNA, 49 bp, synthetic construct
  misc_feature 1..49: note = PDCD1_T01-target
  source 1..49: mol_type = other DNA
tacctctgtggggccatctccctggcccccaaggcgcagatcaaagaga

SEQ ID NO: 99 - DNA, 49 bp, synthetic construct
  misc_feature 1..49: note = TRAC_T01-target
  source 1..49: mol_type = other DNA
ttgtcccacagatatccagaaccctgaccctgccgtgtaccagctgaga

SEQ ID NO: 100 - DNA, 49 bp, synthetic construct
  misc_feature 1..49: note = CD52_T02-target
  source 1..49: mol_type = other DNA
ttcctcctactcaccatcagcctcctggttatggtacaggtaagagcaa

SEQ ID NO: 101 - DNA, 47 bp, synthetic construct
  misc_feature 1..47: note = B2M_T02-target
  source 1..47: mol_type = other DNA
ttagctgtgctcgcgctactctctctttctggcctggaggctatcca

SEQ ID NO: 102 - DNA, 49 bp, synthetic construct
  misc_feature 1..49: note = CIITA_T01-target
  source 1..49: mol_type = other DNA
tctggctgggctgatcttccagcctcccgcccgctgcctgggagcccta

SEQ ID NO: 103 - DNA, 47 bp, synthetic construct
  misc_feature 1..47: note = CS1_target
  source 1..47: mol_type = other DNA
tatatcctttggcagctcacaggtgagtccggccggattctcttcca

SEQ ID NO: 104 - DNA, 1587 bp, synthetic construct
  misc_feature 1..1587: note = MESO-CAR
  source 1..1587: mol_type = other DNA
atggctctgcccgtcaccgctctgctgctgccactggccctgctgctgcacgcagcaaggccacaggtgcagctgcagcagcctggcgcagagctggtgaagcctggcgccagcatgaagctgtcctgcaaggcctctggctacacattcacctcctattggatgcactgggtgaagcagcgcccaggccagggactggagtggatcggcatgatccaccccaactctgacaataccatctactatgagaagtttaagagcaaggccacactgaccgtggataagagctcctctacagcctacatgcagctgagctccctgacctccgaggactctgccgtgtactattgcgccatcatcatcacacccgtggtgcctaagttcgattattggggccagggcaccacactgaccgtgtctagcggaggaggaggaagcggaggaggaggatccggcggcggcggctctgacatcgtgatgacacagagccaccagtttatgagcacctccgtgggcgaccgggtgagcgtgacctgtaaggcctcccacgatgtgggcacctctgtggcctggtaccagcagaagccaggccagagccccaagctgctgatctattgggcctccacaaggcacaccggagtgccagaccgcttcacaggatctggaagcggcaccgacttcaccctgaccatcagcaacgtgcagtccgaggacctggccgattacttctgtcagcagtactcctcttatcctctgacatttggcgcaggaaccaagctggagctgaagagggcctctgatccaggctccggcggaggaggatcctgcccttacagcaacccatccctgtgctctggaggaggaggatcttgtccctatagcaatcctagcctgtgctccggcggaggaggcagcaccacaaccccagcaccaaggccacctacacctgcaccaaccatcgcatcccagccactgtctctgaggccagaggcatgcagacctgcagcaggcggcgccgtgcacaccagaggcctggacttcgcctgcgatatctacatctgggcacctctggcaggaacatgtggcgtgctgctgctgtccctggtcatcaccctgtattgtaagcgaggccggaagaaactgctgtatattttcaaacagccctttatgagacctgtgcagactacccaggaggaagacggctgcagctgtaggttccccgaggaagaggaaggcgggtgtgagctgagggtcaagtttagccgctccgcagatgcccctgcttaccagcaggggcagaatcagctgtataacgagctgaatctgggacggagagaggaatacgacgtgctggataaaaggcgcgggagagaccccgaaatgggaggcaagccacgacggaaaaacccccaggagggcctgtacaatgaactgcagaaggacaaaatggcagaggcctatagtgaaatcgggatgaagggagagagaaggcgcggcaaagggcacgatggcctgtaccaggggctgtctactgccaccaaggacacctatgatgctctgcatatgcaggcactgcctccaaggtga

SEQ ID NO: 105 - DNA, 1566 bp, synthetic construct
  misc_feature 1..1566: note = MUC1-CAR
  source 1..1566: mol_type = other DNA
atggagtggatctggatcttcctgttcatcctgagcggcaccgccggcgtgcagagccaggtgcagctgcagcagagcggcgccgagctggccaggcccggcgccagcgtgaagctgagctgcaaggccagcggctacaccttcaccaactacggcctgagctgggtgaagcagaggaccggccagggcctggagtggatcggcgagaaccaccccggcagcggcatcatctaccacaacgagaagttcaggggcaaggccaccctgaccgccgacaagagcagcagcaccgcctacgtgcagctgagcagcctgaccagcgaggacagcgccgtgtacttctgcgccaggagcagcggcaccaggggcttcgcctactggggccagggcaccctggtgaccgtgagcgccggcggcggcggcagcggcggcggcggcagcggcggcggcggcagcatgaagctgcccgtgaggctgctggtgctgatgttctggatccccgccagcagcagcgacgtgctgatgacccagacccccctgagcctgcccgtgagcctgggcgaccaggccagcatcagctgcaggagcagccagagcatcgtgcacagcaacggcaacacctacctggagtggtacctgcagaagcccggccagagccccaagctgctgatctacaaggtgagcaacaggttcagcggcgtgcccgacaggttcagcggcagcggcagcggcaccgacttcaccctgaagatcagcagggtggaggccgaggacctgggcgtgtactactgcttccagggcagccacggcccctggaccttcggcggcggcaccaagctggagatcaagagggccttcgtgcccgtgttcctgcccgccaagcccaccaccacccccgcccccaggccccccacccccgcccccaccatcgccagccagcccctgagcctgaggcccgaggcctgcaggcccgccgccggcggcgccgtgcacaccaggggcctggacttcgcctgcgacatctacatctgggcccccctggccggcacctgcggcgtgctgctgctgagcctggtgatcaccctgtactgcaaccacaggaacaagaggggcaggaagaagctgctgtacatcttcaagcagcccttcatgaggcccgtgcagaccacccaggaggaggacggctgcagctgcaggttccccgaggaggaggagggcggctgcgagctgagggtgaagttcagcaggagcgccgacgcccccgcctaccagcagggccagaaccagctgtacaacgagctgaacctgggcaggagggaggagtacgacgtgctggacaagaggaggggcagggaccccgagatgggcggcaagcccaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccagg

SEQ ID NO: 106 - DNA, 1440 bp, synthetic construct
  misc_feature 1..1440: note = Trop2-CAR
  source 1..1440: mol_type = other DNA
gacatccagctgacccagagccccagcagcctgagcgccagcgtgggcgacagggtgagcatcacctgcaaggccagccaggacgtgagcatcgccgtggcctggtaccagcagaagcccggcaaggcccccaagctgctgatctacagcgccagctacaggtacaccggcgtgcccgacaggttcagcggcagcggcagcggcaccgacttcaccctgaccatcagcagcctgcagcccgaggacttcgccgtgtactactgccagcagcactacatcacccccctgaccttcggcgccggcaccaaggtggagatcaagggcggcggcggcagcggcggcggcggcagcggcggcggcggcagccaggtgcagctgcagcagagcggcagcgagctgaagaagcccggcgccagcgtgaaggtgagctgcaaggccagcggctacaccttcaccaactacggcatgaactgggtgaagcaggcccccggccagggcctgaagtggatgggctggatcaacacctacaccggcgagcccacctacaccgacgacttcaagggcaggttcgccttcagcctggacaccagcgtgagcaccgcctacctgcagatcagcagcctgaaggccgacgacaccgccgtgtacttctgcgccaggggcggcttcggcagcagctactggtacttcgacgtgtggggccagggcagcctggtgaccgtgagcagcttcgtgcccgtgttcctgcccgccaagcccaccaccacccccgcccccaggccccccacccccgcccccaccatcgccagccagcccctgagcctgaggcccgaggcctgcaggcccgccgccggcggcgccgtgcacaccaggggcctggacttcgcctgcgacatctacatctgggcccccctggccggcacctgcggcgtgctgctgctgagcctggtgatcaccctgtactgcaaccacaggaacaagaggggcaggaagaagctgctgtacatcttcaagcagcccttcatgaggcccgtgcagaccacccaggaggaggacggctgcagctgcaggttccccgaggaggaggagggcggctgcgagctgagggtgaagttcagcaggagcgccgacgcccccgcctaccagcagggccagaaccagctgtacaacgagctgaacctgggcaggagggaggagtacgacgtgctggacaagaggaggggcagggaccccgagatgggcggcaagcccaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccagg

SEQ ID NO: 107 - DNA, 1536 bp, synthetic construct
  misc_feature 1..1536: note = FAP1-41BB-CAR
  source 1..1536: mol_type = other DNA
atggcactgccagtgaccgcactgctgctgccactggcactgctgctgcacgcagcacggcctcaggtgcagctggtgcagagcggagcagaggtgaagaagccaggagcctccgtgaaggtgtcttgcaagacaagcagatacacattcaccgagtataccatccactgggtgaggcaggcacctggacagaggctggagtggatcggaggcatcaaccctaacaatggcatcccaaactacaatcagaagtttaagggcagggtgacaatcaccgtggacacatctgccagcaccgcctatatggagctgagctccctgcgcagcgaggataccgccgtgtactattgcgcccggagaaggatcgcctacggctatgacgagggccacgccatggattactggggccagggcacactggtgaccgtgtctagcggctccacatctggaagcggcaagccaggctccggagagggatctacaaaggacatcgtgatgacccagtctccagatagcctggccgtgtccctgggagagagggcaaccatcaattgtaagtcctctcagagcctgctgtattccagaaaccagaagaattacctggcctggtatcagcagaagcctggccagccccctaagctgctgatcttctgggcatctacaagggagagcggagtgccagacagattttccggctctggcttcggcaccgattttacactgaccatcagctccctgcaggcagaggacgtggcagtgtactattgccagcagtacttctcctatcccctgacatttggccagggcaccaaggtggagatcttcgtgcctgtgtttctgccagcaaagcctaccacaaccccagcaccaaggccaccaacacctgcaccaaccatcgcatcccagccactgtctctgaggcctgaggcatgcaggccagcagcaggaggagcagtgcacacccgcggcctggactttgcctgcgatatctacatctgggcaccactggcaggaacatgtggagtgctgctgctgtccctggtcatcaccctgtactgtaaccaccggaataagcggggcagaaagaagctgctgtatatcttcaagcagcccttcatgaggcccgtgcagacaacccaggaggaggacggctgctcttgtaggttcccagaggaggaggagggaggatgtgagctgcgggtgaagtttagcagatccgcagatgcacctgcataccagcagggacagaaccagctgtataacgagctgaatctgggaagaagggaggagtacgacgtgctggataagagaaggggaagggacccagagatgggaggcaagccaaggaggaagaaccctcaggagggactgtacaatgagctgcagaaggacaagatggccgaggcctattctgagatcggcatgaagggagagagaaggaggggcaagggacacgatggactgtaccagggcctgagcacagccaccaaggacacctatgatgccctgcacatgcaggccctgcctccaagataa

SEQ ID NO: 108 - DNA, 1548 bp, synthetic construct
  misc_feature 1..1548: note = FAP2-41BB-CAR
  source 1..1548: mol_type = other DNA
atggcactgccagtgaccgcactgctgctgccactggcactgctgctgcacgcagcacggcctgaggtgcagctgcagcagtctggacctgagctggtgaagccaggagcatccgtgcgcatgtcttgtaaggccagcggctacaccttcacagattactatatgaagtgggtgaagcagtccctgggcaagtctctggagtggatcggcgacatctatcccaacaatggcgagatcccttacaaccagaagtttaagggcaaggccaccctgacagccgacaagacaagctccaccgcctatatgcagctgaatagcctgacctccgaggattctgccgtgtactattgcgtgagaggctactattacggactggcaatggactactggggacagggcacaagcgtgacctccgtggtgggaggaggaggctccggaggaggaggctctggaggcggcggcagccaggcagtggtgacacaggagagcgccctgacctccccaggagagaccgtgacactgacctgtcggtctagcacaggcgccgtgaccacaagcaactacgccaattgggtgcaggagaagcctgatagactgttcacaggcctgatcggcgccaccaacaatagggcaccaggagtgccagcacgcttttccggatctctgatcggcgacaaggccgccctgacaatcaccggagcacagaccgaggatgaggccatctatttctgcgccctgtggtacagcaaccacttcatctttggctccggcacaaaggtgaccgtgctgggcggcggcggcagcggcggcggcggctccggcggcggcggcagcttcgtgcctgtgtttctgccagcaaagcctaccacaaccccagcaccaaggccaccaacacctgcaccaaccatcgcatcccagccactgtctctgaggcctgaggcatgcaggccagcagcaggaggagcagtgcacacccgcggcctggactttgcctgcgatatctacatctgggcaccactggcaggaacatgtggagtgctgctgctgtccctggtcatcaccctgtactgtaaccaccggaataagcggggcagaaagaagctgctgtatatcttcaagcagcccttcatgaggcccgtgcagacaacccaggaggaggacggctgctcttgtaggttcccagaggaggaggagggaggatgtgagctgcgggtgaagtttagcagatccgcagatgcacctgcataccagcagggacagaaccagctgtataacgagctgaatctgggaagaagggaggagtacgacgtgctggataagagaaggggaagggacccagagatgggaggcaagccaaggaggaagaaccctcaggagggactgtacaatgagctgcagaaggacaagatggccgaggcctattctgagatcggcatgaagggagagagaaggaggggcaagggacacgatggactgtaccagggcctgagcacagccaccaaggacacctatgatgccctgcacatgcaggccctgcctccaagataa

SEQ ID NO: 109 - DNA, 1563 bp, synthetic construct
  misc_feature 1..1563: note = FAP3-41BB-CAR
  source 1..1563: mol_type = other DNA
atggcactgccagtgaccgcactgctgctgccactggcactgctgctgcacgcagcacggcctcaggtgcagctggtgcagtctggagcagaggtgaagaagccaggcgcctctgtgaaggtgagctgtaaggcctccggctatacctttacagagaacatcatccactgggtgcgccaggcacctggacagggactggagtggatgggctggttccacccaggctctggcagcatcaagtacaatgagaagtttaaggaccgggtgaccatgacagccgatacctccacatctaccgtgtatatggagctgagctccctgagatccgaggacacagccgtgtactattgcgcaaggcacggaggaaccggaaggggagccatggattactggggccagggcacactggtgaccgtgtctagcggaggaggaggctccggaggaggaggatctggcggcggcggcagcgacatccagatgacacagtccccatcctctctgagcgcctccgtgggcgatagggtgacaatcacctgtagagcctctaagagcgtgtccacctctgcctacagctatatgcactggtaccagcagaagccaggcaaggcccccaagctgctgatctatctggcctctaacctggagagcggagtgccttcccggttcagcggctccggatctggaacagactttacactgaccatcagctccctgcagccagaggatttcgccacctactattgccagcacagcagggagctgccctacacatttggccagggcaccaagctggagatcaagaggggcggcggcggctccggcggaggaggctccggaggcggcggcagcttcgtgcctgtgtttctgccagcaaagcctaccacaaccccagcaccaaggccaccaacacctgcaccaaccatcgcatcccagccactgtctctgaggcctgaggcatgcaggccagcagcaggaggagcagtgcacacccgcggcctggactttgcctgcgatatctacatctgggcaccactggcaggaacatgtggagtgctgctgctgtccctggtcatcaccctgtactgtaaccaccggaataagcggggcagaaagaagctgctgtatatcttcaagcagcccttcatgaggcccgtgcagacaacccaggaggaggacggctgctcttgtaggttcccagaggaggaggagggaggatgtgagctgcgggtgaagtttagcagatccgcagatgcacctgcataccagcagggacagaaccagctgtataacgagctgaatctgggaagaagggaggagtacgacgtgctggataagagaaggggaagggacccagagatgggaggcaagccaaggaggaagaaccctcaggagggactgtacaatgagctgcagaaggacaagatggccgaggcctattctgagatcggcatgaagggagagagaaggaggggcaagggacacgatggactgtaccagggcctgagcacagccaccaaggacacctatgatgccctgcacatgcaggccctgcctccaagataa

SEQ ID NO: 110 - DNA, 1542 bp, synthetic construct
  misc_feature 1..1542: note = FAP4-41BB-CAR
  source 1..1542: mol_type = other DNA
atggcactgccagtgaccgcactgctgctgccactggcactgctgctgcacgcagcacggcctgaggtgcagctgctggagagcggcggcggcctggtgcagcccggcggcagcctgaggctgagctgcgccgccagcggcttcaccttcagcagctacgccatgagctgggtgaggcaggcccccggcaagggcctggagtgggtgagcgccatcatcggcagcggcgccagcacctactacgccgacagcgtgaagggcaggttcaccatcagcagggacaacagcaagaacaccctgtacctgcagatgaacagcctgagggccgaggacaccgccgtgtactactgcgccaagggctggttcggcggcttcaactactggggccagggcaccctggtgaccgtgagcagcggaggaggaggctccggaggaggaggatctggcggcggcggcagcggcggcggcggctccggcggaggaggctccggaggcggcggcagcgagatcgtgctgacccagagccccggcaccctgagcctgagccccggcgagagggccaccctgagctgcagggccagccagagcgtgaccagcagctacctggcctggtaccagcagaagcccggccaggcccccaggctgctgatcaacgtgggcagcaggagggccaccggcatccccgacaggttcagcggcagcggcagcggcaccgacttcaccctgaccatcagcaggctggagcccgaggacttcgccgtgtactactgccagcagggcatcatgctgccccccaccttcggccagggcaccaaggtggagatcaagttcgtgcctgtgtttctgccagcaaagcctaccacaaccccagcaccaaggccaccaacacctgcaccaaccatcgcatcccagccactgtctctgaggcctgaggcatgcaggccagcagcaggaggagcagtgcacacccgcggcctggactttgcctgcgatatctacatctgggcaccactggcaggaacatgtggagtgctgctgctgtccctggtcatcaccctgtactgtaaccaccggaataagcggggcagaaagaagctgctgtatatcttcaagcagcccttcatgaggcccgtgcagacaacccaggaggaggacggctgctcttgtaggttcccagaggaggaggagggaggatgtgagctgcgggtgaagtttagcagatccgcagatgcacctgcataccagcagggacagaaccagctgtataacgagctgaatctgggaagaagggaggagtacgacgtgctggataagagaaggggaagggacccagagatgggaggcaagccaaggaggaagaaccctcaggagggactgtacaatgagctgcagaaggacaagatggccgaggcctattctgagatcggcatgaagggagagagaaggaggggcaagggacacgatggactgtaccagggcctgagcacagccaccaaggacacctatgatgccctgcacatgcaggccctgcctccaagataa

SEQ ID NO: 111 - DNA, 1908 bp, synthetic construct
  misc_feature 1..1908: note = LHA-2A-FAPCAR-RHA
  source 1..1908: mol_type = other DNA
aagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagggcagcggcgtgaagcagacactgaacttcgacctgctgaagctggcaggcgatgtggagtccaatccaggacctatggcactgccagtgaccgcactgctgctgccactggcactgctgctgcacgcagcacggcctcaggtgcagctggtgcagagcggagcagaggtgaagaagccaggagcctccgtgaaggtgtcttgcaagacaagcagatacacattcaccgagtataccatccactgggtgaggcaggcacctggacagaggctggagtggatcggaggcatcaaccctaacaatggcatcccaaactacaatcagaagtttaagggcagggtgacaatcaccgtggacacatctgccagcaccgcctatatggagctgagctccctgcgcagcgaggataccgccgtgtactattgcgcccggagaaggatcgcctacggctatgacgagggccacgccatggattactggggccagggcacactggtgaccgtgtctagcggctccacatctggaagcggcaagccaggctccggagagggatctacaaaggacatcgtgatgacccagtctccagatagcctggccgtgtccctgggagagagggcaaccatcaattgtaagtcctctcagagcctgctgtattccagaaaccagaagaattacctggcctggtatcagcagaagcctggccagccccctaagctgctgatcttctgggcatctacaagggagagcggagtgccagacagattttccggctctggcttcggcaccgattttacactgaccatcagctccctgcaggcagaggacgtggcagtgtactattgccagcagtacttctcctatcccctgacatttggccagggcaccaaggtggagatcttcgtgcctgtgtttctgccagcaaagcctaccacaaccccagcaccaaggccaccaacacctgcaccaaccatcgcatcccagccactgtctctgaggcctgaggcatgcaggccagcagcaggaggagcagtgcacacccgcggcctggactttgcctgcgatatctacatctgggcaccactggcaggaacatgtggagtgctgctgctgtccctggtcatcaccctgtactgtaaccaccggaataagcggggcagaaagaagctgctgtatatcttcaagcagcccttcatgaggcccgtgcagacaacccaggaggaggacggctgctcttgtaggttcccagaggaggaggagggaggatgtgagctgcgggtgaagtttagcagatccgcagatgcacctgcataccagcagggacagaaccagctgtataacgagctgaatctgggaagaagggaggagtacgacgtgctggataagagaaggggaagggacccagagatgggaggcaagccaaggaggaagaaccctcaggagggactgtacaatgagctgcagaaggacaagatggccgaggcctattctgagatcggcatgaagggagagagaaggaggggcaagggacacgatggactgtaccagggcctgagcacagccaccaaggacacctatgatgccctgcacatgcaggccctgcctccaaga

SEQ ID NO: 112 - DNA, 3814 bp, synthetic construct
  misc_feature 1..3814: note = LHA-2A-MESOCAR-EF1a-DLNGFR-RHA
  source 1..3814: mol_type = other DNA
gactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccggcagcggcgtgaagcagaccctgaacttcgacctgctgaagctggcaggcgatgtggagtccaatccaggacctatggctctgcccgtcaccgctctgctgctgccactggccctgctgctgcacgcagcaaggccacaggtgcagctgcagcagcctggcgcagagctggtgaagcctggcgccagcatgaagctgtcctgcaaggcctctggctacacattcacctcctattggatgcactgggtgaagcagcgcccaggccagggactggagtggatcggcatgatccaccccaactctgacaataccatctactatgagaagtttaagagcaaggccacactgaccgtggataagagctcctctacagcctacatgcagctgagctccctgacctccgaggactctgccgtgtactattgcgccatcatcatcacacccgtggtgcctaagttcgattattggggccagggcaccacactgaccgtgtctagcggaggaggaggaagcggaggaggagaatccggcggcggcggctctgacatcgtgatgacacagagccaccagtttatgagcacctccgtgggcgaccgggtgagcgtgacctgtaaggcctcccacgatgtgggcacctctgtggcctggtaccagcagaagccaggccagagccccaagctgctgatctattgggcctccacaaggcacaccggagtgccagaccgcttcacaggatctggaagcggcaccgacttcaccctgaccatcagcaacgtgcagtccgaggacctggccgattacttctgtcagcagtactcctcttatcctctgacatttggcgcaggaaccaagctggagctgaagagggcctctgatccaggctccggcggaggagaatcctgcccttacagcaacccatccctgtgctctggaggaggaggatcttgtccctatagcaatcctagcctgtgctccggcggaggaggcagcaccacaaccccagcaccaaggccacctacacctgcaccaaccatcgcatcccagccactgtctctgaggccagaggcatgcagacctgcagcaggcggcgccgtgcacaccagaggcctggacttcgcctgcgatatctacatctgggcacctctggcaggaacatgtggcgtgctgctgctgtccctggtcatcaccctgtattgtaagcgaggccggaagaaactgctgtatattttcaaacagccctttatgagacctgtgcagactacccaggaggaagacggctgcagctgtaggttccccgaggaagaggaaggcgggtgtgagctgagggtcaagtttagccgctccgcagatgcccctgcttaccagcaggggcagaatcagctgtataacgagctgaatctgggacggagagaggaatacgacgtgctggataaaaggcgcgggagagaccccgaaatgggaggcaagccacgacggaaaaacccccaggagggcctgtacaatgaactgcagaaggacaaaatggcagaggcctatagtgaaatcgggatgaagggagagagaaggcgcggcaaagggcacgatggcctgtaccaggggctgtctactgccaccaaggacacctatgatgctctgcatatgcaggcactgcctccaaggtgacgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagtcgacgccaccatgggggcaggtgccaccggccgcgccatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgatgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggtcttgtggcctacatagccttcaagaggtgagcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtacggcttgattatgatcaggcgcagatcaaagagagcctgcgggcagagctcagggtgacaggtgcggcctcggaggccccggggcaggggtgagctgagccggtcctggggtgggtgtcccctcctgcacaggatcaggagctccagggtcgtagggcagggaccccccagctccagtccagggctctgtcctgcacctggggaatggtgaccggcatctctgtcctctagctctggaagcaccccagcccctctagtctgccctcacccctgaccctgaccctccaccctgaccccgtcctaacccctgacctttg

SEQ ID NO: 113 - DNA, 2736 bp, synthetic construct
  misc_feature 1..2736: note = EF1A-FAPCAR
  source 1..2736: mol_type = other DNA
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagtcgacgccaccatggcactgccagtgaccgcactgctgctgccactggcactgctgctgcacgcagcacggcctcaggtgcagctggtgcagagcggagcagaggtgaagaagccaggagcctccgtgaaggtgtcttgcaagacaagcagatacacattcaccgagtataccatccactgggtgaggcaggcacctggacagaggctggagtggatcggaggcatcaaccctaacaatggcatcccaaactacaatcagaagtttaagggcagggtgacaatcaccgtggacacatctgccagcaccgcctatatggagctgagctccctgcgcagcgaggataccgccgtgtactattgcgcccggagaaggatcgcctacggctatgacgagggccacgccatggattactggggccagggcacactggtgaccgtgtctagcggctccacatctggaagcggcaagccaggctccggagagggatctacaaaggacatcgtgatgacccagtctccagatagcctggccgtgtccctgggagagagggcaaccatcaattgtaagtcctctcagagcctgctgtattccagaaaccagaagaattacctggcctggtatcagcagaagcctggccagccccctaagctgctgatcttctgggcatctacaagggagagcggagtgccagacagattttccggctctggcttcggcaccgattttacactgaccatcagctccctgcaggcagaggacgtggcagtgtactattgccagcagtacttctcctatcccctgacatttggccagggcaccaaggtggagatcttcgtgcctgtgtttctgccagcaaagcctaccacaaccccagcaccaaggccaccaacacctgcaccaaccatcgcatcccagccactgtctctgaggcctgaggcatgcaggccagcagcaggaggagcagtgcacacccgcggcctggactttgcctgcgatatctacatctgggcaccactggcaggaacatgtggagtgctgctgctgtccctggtcatcaccctgtactgtaaccaccggaataagcggggcagaaagaagctgctgtatatcttcaagcagcccttcatgaggcccgtgcagacaacccaggaggaggacggctgctcttgtaggttcccagaggaggaggagggaggatgtgagctgcgggtgaagtttagcagatccgcagatgcacctgcataccagcagggacagaaccagctgtataacgagctgaatctgggaagaagggaggagtacgacgtgctggataagagaaggggaagggacccagagatgggaggcaagccaaggaggaagaaccctcaggagggactgtacaatgagctgcagaaggacaagatggccgaggcctattctgagatcggcatgaagggagagagaaggaggggcaagggacacgatggactgtaccagggcctgagcacagccaccaaggacacctatgatgccctgcacatgcaggccctgcctccaagataa

SEQ ID NO: 114 - DNA, 30 bp, synthetic construct
  misc_feature 1..30: note = NFAT responsive element
  source 1..30: mol_type = other DNA
ggaggaaaaactgtttcatacagaaggcgt

SEQ ID NO: 115 - DNA, 32 bp, synthetic construct
  misc_feature 1..32: note = NFAT responsive element (bis)
  source 1..32: mol_type = other DNA
ggaggaaaaactgtttcatacacagaaggcct